(19) United States
(12) Patent Application Publication
(10) Pub. No.: US 2024/0318242 A1
(43) Pub. Date: Sep. 26, 2024

(54) METHODS AND SYSTEMS FOR MEASURING MULTIPLEX RNA EXPRESSION

(71) Applicant: City University of Hong Kong, Kowloon (HK)

(72) Inventors: Zongli Zheng, Kowloon (HK); Chenyu Lu, Kowloon (HK)

(21) Appl. No.: 18/245,599

(22) PCT Filed: Jan. 28, 2023
(86) PCT No.: PCT/CN2023/073581

§ 371 Date: Mar. 16, 2023

Related U.S. Application Data
(60) Provisional application No. 63/304,489, filed on Jan. 28, 2022.

(51) Int. Cl. C12Q 1/6869; C12Q 1/6806; C12Q 1/686

(52) U.S. Cl. CPC C12Q 1/6869; C12Q 1/6806; C12Q 1/686; G01N 2800/52

(57) ABSTRACT

A method of measuring RNA expression level of a target sequence in a sample is performed by simultaneously enriching the target sequence and corresponding RNA and calculating a RNA-to-DNA ratio of the same target sequence. A method of treating cancer includes the step of using the method of measuring RNA expression level of a target sequence. A kit includes components for performing the method of measuring RNA expression level of a target sequence.

### FIELD OF THE INVENTION

The present invention relates to next-generation sequencing. More specifically, the present invention relates to methods and systems for measuring multiplex RNA expression.

### BACKGROUND

Clinics widely use next-generation Sequencing (NGS) technology for many molecular diagnosis assays but not RNA expression. However, there are currently no reliable methods for multiplexing RNA quantification using NGS.

Currently-available RNA expression by RNA sequencing suffers from i) low efficiency in quantifying clinically relevant genes; ii) low accuracy in measuring low-level but clinically important genes, such as ALK—the most successful target in precision medicine; iii) high cost, as it allows only whole genome analysis and the vast majority of which are not clinically relevant; iv) not applicable to the most accessible clinical samples such as FFPE tumor tissues. Other non-NGS methods in RNA expression analyses generally associated with low multiplexing ability (e.g. RT-qPCR or digital PCR) and additional sample and workflow are needed (e.g. RNA-Scope and NanoString), in addition to the main stream NGS workflow.

Revolutionary cancer immunotherapy using immune checkpoint inhibitors (ICIs) may bring long-lasting responses in multiple cancers. However, current diagnosis methods, to find the about 20-30% of cancer patients who could benefit from ICI therapies, are suboptimal.

Researchers spend great effort to identify predictive markers for ICIs, including PD-L1 expression, tumor-infiltrating lymphocytes (TIL), especially the CD⁸⁺ T cells⁵. Higher ORR was correlated with high expression of PD-L1, ORR for PD-L1 tumour proportion score (TPS) of 1% or greater patients and TPS 50% or greater patients was 27% and 39%, respectively⁶ Currently, four PD-L1 IHC assay^{7,8} (22C3, 28-8, SP142, and SP263) was approved by FDA as companion or complementary diagnostic assay for different ICIs (pembrolizumab, nivolumab, atezolizumab and durvalumab, respectively) in NSCLC. However, the cutoffs determining high PD-L1 expression vary among different studies⁷ (1%, 5%, 10%, or 50% PD-L1 positive cells). Also, the current IHC assays recognize different epitopes of PD-L1. Ventana SP142 and SP263 target the cytoplasmic domain of PD-L1, while 22C3 and 28-8 clones are raised against epitopes within the extracellular domain⁹, which might explain the dis-concordance between the SP142/SP263 and 22C3/28-8⁸. Thus, this semi-quantified method for ICI response prediction is imperfect, highlighting the necessity in developing more precise method for PD-L1 quantification.

In view of the above, there remains a need to develop new method for detecting RNA expressions precisely and quickly. Also, there is a need of identifying predictive markers for ICIs, which would be critical for precision medicine.

### SUMMARY OF THE INVENTION

An embodiment of the present invention relates to method of measuring RNA expression level of a target sequence in a sample by simultaneously enriching the target sequence and corresponding RNA and calculating a RNA-to-DNA ratio of the same target sequence, including the steps of
(a) reverse transcribing RNA into complementary DNA (cDNA) resulting in a mixture of cDNA and genomic DNA (gDNA);
(b) ligating a universal sequencing adaptor to a terminal of the gDNA and cDNA from step (a);
(c) conducting a first polymerase chain reaction process to produce a single-stranded primer extension product including a complementary sequence, wherein the complementary sequence is complementary to the target sequence on the gDNA, the cDNA, or a combination thereof, and is complementary to the universal sequencing adaptor;
(d) conducting a second polymerase chain reaction process to amplify the single-stranded primer extension product; and
(e) performing a next-generation sequencing process and calculating the relative ratio of cDNA sequencing reads to gDNA sequencing reads, namely the RNA-to-DNA ratio, of the same target sequence,
   wherein the target sequence is selected from a group of a gene sequence, an exon of a gene, and a combination thereof.

An embodiment of the present invention relates to a method of treating cancer in a subject containing the steps of testing for RNA expression level of a target sequence by using the method of measuring RNA expression level as provided herein, and applying an immune checkpoint inhibitor to the subject.

Another embodiment of the present invention relates to a kit for performing the method of measuring RNA expression level of a target sequence in a sample by simultaneously enriching a target DNA and corresponding RNA and calculating a RNA-to-DNA ratio of the same target sequence as described herein, the kit includes:
(a) a reverse transcriptase for transcribing RNA into complementary DNA (cDNA);
(b) a universal sequencing adaptor to be ligated to a terminal of gDNA and cDNA in the sample;
(c) a first primer that binds to the cDNA and gDNA sequence at a position that is 3′ in relation to the target sequence on the cDNA or gDNA; and
(d) a second primer that binds to the target sequence at a position that is between the first primer and the target sequence on the cDNA or gDNA.

### BRIEF DESCRIPTION OF THE DRAWINGS

FIG. 1a shows a schematic description of the anchored multiplex PCR;

FIG. 1b shows a schematic description of Anchored Ligation Priming for Highly Accurate RNA expression (Alpha-RNA), gene specific primers (GSP1s) and their 3′ nested primers GSP2 near the exon/intron boundary;

FIG. 2a shows a linear regression between expression data from sense primer and antisense primer (left: B2M, right: CHMP2A);

FIG. 2b shows a linear regression between exon3 expression data of CHMP2A and exon6 expression data;

FIG. 2c shows a graph of RNA expression of ALK in fusion-positive samples (left) and fusion-negative samples (right);

FIG. 2d shows a correlation between RNA expression of CD274 (encoding PD-L1) exons 3, 4, and 6 and PDCD1 (encoding PD-1) and the protein expression of PD-L1;

FIG. 3a shows Kaplan-Meier survival curves of NSCLC patients with PD-L1 immunohistochemistry staining of tumor tissue showing >=50% positive cells (n=34) and with <50% positive cells (n=138);

FIG. 3b shows Kaplan-Meier survival curves of NSCLC patients with PD-L1 protein expression by immunohistochemistry (IHC) staining of tumor tissue showing any positive cells, i.e., >0% (‘PD L1.0=2’; n=135) and no positive cells, i.e., 0% (‘PD L1.0=1’; n=37);

FIG. 3c shows Kaplan-Meier survival curves of NSCLC patients with PD-L1 protein expression by immunohistochemistry (IHC) staining of tumor tissue showing >=1% positive cells (‘PD_L1.1=2’; n=83) and no positive cells (‘PD_L1.1=1’; n=89);

FIG. 3d shows Kaplan-Meier survival curves of NSCLC patients with PD-L1 protein 10 expression by immunohistochemistry (IHC) staining of tumor tissue showing >=5% positive cells (‘PD_L1.5=2’; n=72) and no positive cells (‘PD_L1.5=1’; n=100);

FIG. 3e shows Kaplan-Meier survival curves of NSCLC patients with PD-L1 protein expression by immunohistochemistry (IHC) staining of tumor tissue showing >=10% positive cells (‘PD_L1.10=2’; n=57) and no positive cells (‘PD_L1.10=1’; n=115);

FIG. 3f shows Kaplan-Meier survival curves of NSCLC patients with PD-L1 protein expression by immunohistochemistry (IHC) staining of tumor tissue showing >=25% positive cells (‘PD_L1.25=2’; n=41) and no positive cells (‘PD_L1.25=1’; n=131);

FIG. 3g shows Kaplan-Meier curves for survival of NSCLC patients with RNA expression by the Alpha-RNA method showing CD274 exon6 high (n=40) and CD274 exon6 low (n=117);

FIG. 3h shows Kaplan-Meier curves for survival of NSCLC patients with RNA expression by the Alpha-RNA method showing CD274 exon6 high and exon4 low (n=8), CD274 exon6 high and exon4 high (n=30) and CD274 exon6 low (“others”; n=117);

FIG. 4a shows the expression of CD274 (different exons) in TCGA cancer types;

FIG. 4b shows the Cox regression-computed Hazard Ratio (x-axis) for death in CD274 exon high expression patients versus CD274 low expression patients by the statistical significance difference (y-axis; −log 10 transformed P value);

FIG. 4c shows Kaplan-Meier curves for survival of TCGANSCLC patients with CD274 exon3 high expression and exon5 low expression (n=26), CD274 with CD274 exon3 high expression and exon5 high expression (n=263) and CD274 exon3 low expression (n=674);

FIG. 5a shows Kaplan-Meier curves for progression-free survival of NSCLC patients, after receiving immune-checkpoint inhibitor therapy, with RNA expression by the Alpha-RNA method showing CD274 high expression and GZMA low expression (n=12), CD274 high expression and GZMA high expression (n=4) and CD274 low expression (n=7); and

FIG. 5b shows Kaplan-Meier curves for survival of NSCLC patients, after receiving immune-checkpoint inhibitor therapy, with RNA expression by the Alpha-RNA method showing CD274 high expression and PRF1 low expression (n=12), CD274 high expression and PRF1 high expression (n=3) and CD274 low expression (n=7).

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

As used herein, overall survival (OS) is defined as the period from surgery of a patient to the date of the last follow-up or death of the patient.

An embodiment of the present invention relates to a method of measuring RNA expression level of a target sequence in a sample by simultaneously enriching a target DNA and corresponding RNA and calculating a RNA-to-DNA ratio of the same target sequence, including the steps of
(a) reverse transcribing RNA into complementary DNA (cDNA) resulting in a mixture of cDNA and genomic DNA (gDNA);
(b) ligating a universal sequencing adaptor to a terminal of the gDNA and cDNA from step (a);
(c) conducting a first polymerase chain reaction process to produce a single-stranded primer extension product including a complementary sequence, wherein the complementary sequence is complementary to the target sequence on the gDNA, the cDNA, or a combination thereof, and is complementary to the universal sequencing adaptor;
(d) conducting a second polymerase chain reaction process to amplify the single-stranded primer extension product; and
(e) performing a next-generation sequencing process and calculating the relative ratio of cDNA sequencing reads to gDNA sequencing reads (namely RNA-to-DNA ratio) of the same target sequence.

The RNA-to-DNA ratio as mentioned above represents the quantity of RNA expression level per copy gDNA. The description covers both 5′ terminal of a single-stranded gDNA/cDNA and either end or both ends of a double-stranded gDNA/cDNA. In some embodiments, if ligation to single-stranded gDNA and cDNA, then the universal sequencing adaptor is ligated to the 5′ terminal of the gDNA and cDNA. In some embodiments, if ligation to double-stranded gDNA and cDNA, then the universal sequencing adaptor is ligated to either terminal or both terminals of the gDNA and cDNA.

The present application develops a method using Anchored Ligation Priming for Highly Accurate RNA expression (Alpha-RNA) for accurate quantification of multiplex gene RNA expression in biospecimen. Without intending to be bound by theory, it is believed that Alpha-RNA is a novel method based on next-generation sequencing (NGS), and can be used for clinical samples of varied qualities, including highly degraded RNA from formalin-fix paraffin-embedded tissues, and thus is broadly applicable clinically. For example, in cancer diagnosis, Alpha-RNA can accurately quantify those genes relevant in cancer immunity, including PD-1/PD-L1 and immune microenvironment genes such as granzyme and perforin and beyond. Alpha-RNA can be used to guide cancer immunotherapy.

In an embodiment herein, the first polymerase chain reaction process includes the steps of:
(i) contacting a first primer that binds to the cDNA and gDNA sequence at a position that is 3′ in relation to the target sequence on the cDNA or gDNA;
(ii) extending by polymerization the first primer till the terminal including the ligated universal sequencing adaptor to produce the single-stranded primer extension product;
(iii) dissociating the single-stranded primer extension product including the complementary sequence to the target sequence from the template strand cDNA or gDNA; and
(iv) optionally, repeating steps (i) through (iii) one or more times.

In an embodiment herein, the second polymerase chain reaction process includes the steps of binding a second primer to the sequence at a position that is between the first primer and the target sequence on the cDNA or gDNA. In an embodiment herein, the second polymerase chain reaction process further includes the steps of binding a universal primer to the adaptor complementary sequence.

In an embodiment herein, the above steps (i) through (iii) are repeated for from about 1 cycle to about 100 cycles. For example, steps (i) through (iii) are repeated for about 2 cycles to about 100 cycles, or about 5 cycles to about 50 cycles, for example, about 8 cycles, about 9 cycles, about 10 cycles, about 20 cycles, about 30 cycles, about 40 cycles, about 50 cycles, about 60 cycles, about 70 cycles, about 80 cycles about 90 cycles or about 100 cycles.

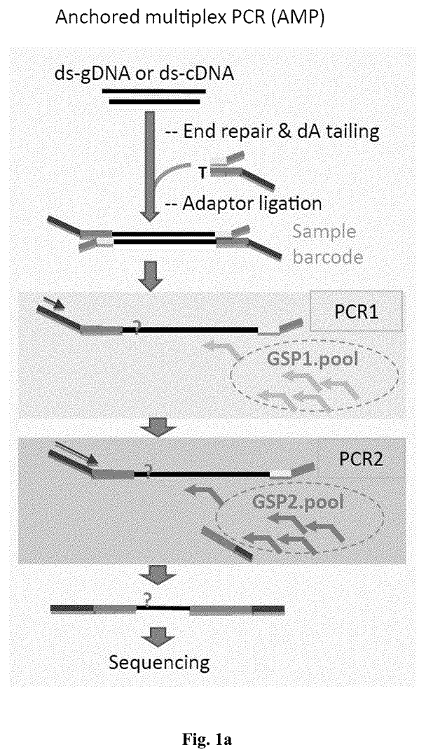

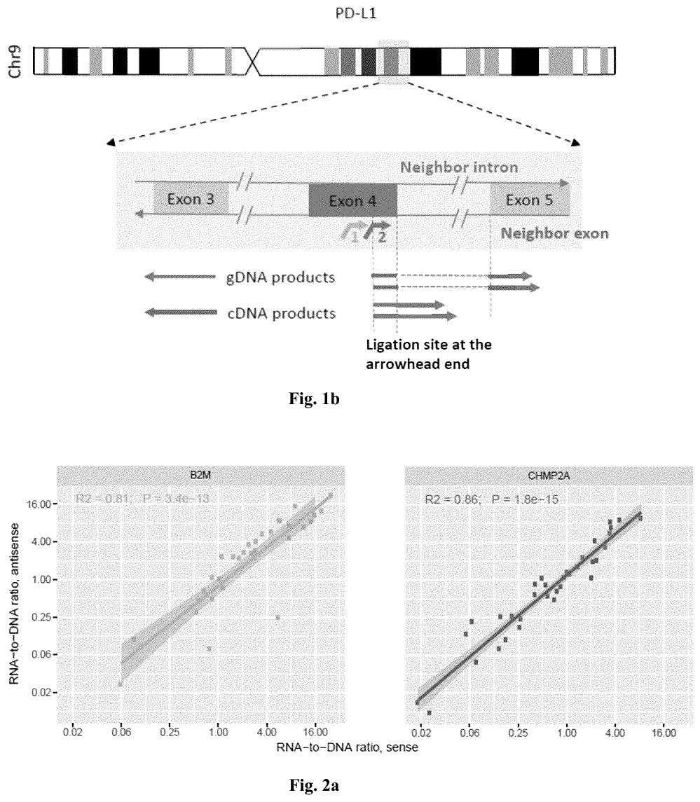

In an embodiment herein, the second primer is used to prime and enrich both gDNA and cDNA. Without intending to be bound by theory, it is believed that this allows accurate calculation of cDNA-to-gDNA ratio for the same primer because of primer efficiency is then not an affecting factor (in other words ‘cancelled out’), and the affection of different efficiencies caused by using two different primers, namely one for gDNA and another for cDNA, may be excluded. For example, as shown in FIG. 1b, at the presence of both genomic DNA and cDNA, a single primer near the exon boundary can prime and enrich both genomic DNA (containing exon and intron) and cDNA (spanning enamoring exons).

In an embodiment herein, the sample is selected from the group of a formalin-fix paraffin-embedded (FFPE) tissue, fresh tissue collected by surgical biopsy or needle aspiration, blood, urine, ascites, pleural effusion, cerebrospinal fluid, pancreas cyst fluid, and a combination thereof. In some embodiments, the sample for testing is selected from the group of a formalin-fix paraffin-embedded tissue, fresh tissue collected by surgical biopsy or needle aspiration, and a combination thereof. In some embodiments, the sample for testing is selected from liquid biopsies such as blood, urine, ascites, pleural effusion, cerebrospinal fluid, pancreas cyst fluid, and a combination thereof. In certain embodiment, the sample could be FFPE tumor tissues to which currently available RNA expression by RNA sequencing is not applicable. In certain embodiment, the sample could be needle biopsy tissues to which the amount of material is limited and currently available RNA expression by RNA sequencing is not applicable.

In an embodiment herein, the method is for quantifying the expression of the target sequence selected from those genes related to immune microenvironment, T-cell functions, antigen presentation, cytotoxic factors, DNA damage repair, immune cell adhesion and migration, housekeeping genes, and other disease relevant genes. For example, in some embodiments, the target sequence can be the immune microenvironment selected from the group of CD274, PDCD1, GZMA, PRF1, and a combination thereof. In some embodiments, the target sequence can be T-cell functions selected from the group of CD3D, CD3E, CD3G, CD6, CD8A, CD8B, FOXP3, SH2D1A, TBX21, TRAT1, and a combination thereof. In some embodiments, the target sequence can be the antigen presentation selected from the group of ATF3, B2M, CCR5, CD1C, CD36, CD4, CD74, CD8A, CD8B, CDC20, CTSS, CTSW CDIC1, CYBB, DTX3L, FCGR1A, HLA genes, IFNG, IRF8, ITGAV, MRC1, PSMB10, PSMB5, PSMB8, PSMB9, PSMC4, SOCS1, TAP1, TAP2, TAPBP, THBD, TNF, TRIM21, UBA7, UBB, UBE2C, ULBP2, VHL, and a combination thereof. In some embodiments, the target sequence can be cytotoxic factors selected from the group of BBC3, CBLC, CD47, CNTFR, FASLG, GHR, GNLY, GZMA, GZMB, GZMH, GZMK, GZAM, IFI16, IFI27, IFI35, IFI6, IFIH1, IFIT1, IFIT2, IFIT3, IFITM1, IFITM2, IFNG, IGF2R, IL11RA, IL12RB2, IL22RA1, IRF1, IRF4, IRF9, ISG15, JAK1, JAK2, JAK3, KIR2DL3, KIR3DL1, KIR3DL2, KLRB1, KLRD1, KLRK1, LIF, MX1, OAS1, OAS2, OAS3, PRF1, PRLR, SIRPA, SPRY4, STAT1, STAT2, TNFSF10, and a combination thereof. In some embodiments, the target sequence can be DNA damage repair selected from the group of ATM, BLM, BRCA1, BRCA2, BRIP1, CCNA1, CCNO, CDK2, DDB2, ERCC3, EXO1, FANCA, H2AFX, ISG15, MGML MLH1, MSH2, MSH6, NBN, NEIL1, PARP4, PIAS4, PMS2, POLD1, POLR2A, RAD50, RAD51, RAD51C, TNKS, TP53, UBA7, UBB, UBE2L XCL1/2, and a combination thereof. In some embodiments, the target sequence can be immune cell adhesion and migration selected from the group of CD2, CD274, CD276, CD28, CD4, CD40, CD40LG, CD58, CD6, CD80, CD86, CD8A, CD8B, CDH1, CDH2, CDH5, CLEC14A, CLEC4E, CLEC5A, CLEC7A, CLECL1, CTLA4, CTNNB1, CXCL12, CXCR4, CYBB, ICAM1, ICAM2, ICAM3, ICAMS, ICOS, ICOSLG, ITGA1, ITGA2, ITGA4, ITGA6, ITGAE, ITGAL, ITGAM, ITGAV ITGAX, ITGB2, ITGB3, ITGB8, MIP9, NCAM1, NECTIN1, NECTIN2, PDCD1, PDCD1LG2, PECAM1, PIK3CA, PIK3CD, PIK3CG, PIK3R1, PIK3R2, PIK3R5, PRKCA, PTPN11, PTPRC, PVR, ROCK1, SELE, SELL, SELP, SIGLEC1, THY1, TIGIT VCAM1, VCAN, VTCN1, and a combination thereof. In some embodiments, the target sequence can be housekeeping genes selected from the group of CHMP2A, GAPDH, B2M, and a combination thereof. In some embodiments, the target sequence is selected from genes related to immune microenvironment, oncogenes, tumor suppressor genes, and a combination thereof.

In an embodiment herein, total nucleic acid is used to perform the reverse transcription in step (a) without removing genomic DNA. Without intending to be bound by theory, it is believed that the gDNA can serve as a perfect internal reference to calculate mRNA per genomic DNA copy.

In an embodiment herein, the first primer has a sequence selected from the group of SEQ ID NOs. 1-379, and analogous thereof with an identity of at least about 80%, at least about 90%, at least about 95%, at least about 98%, or at least about 99% to about 100%, of any one of SEQ ID NOs. 1-379. In some embodiments, the first primer can have a sequence selected from any one of SEQ ID NOs. 1-379, and analogous thereof with an identity of at least about 90%. In some embodiments, the first primer can have a sequence selected from any one of SEQ ID NOs. 1-379, and analogous thereof with an identity of at least about 98%. In some embodiments, the first primer can have a sequence selected from any one of SEQ ID NOs. 1-379.

In an embodiment herein, the second primer has a sequence selected from the group consisting of SEQ ID NOs. 380-758, and analogous thereof with an identity of at least about 80% at least about 90%, at least about 95%, at least about 98%, or at least about 99% to about 100%, of any one of SEQ ID NOs. 380-758. In some embodiments, the first primer can have a sequence selected from any one of SEQ ID NOs. 380-758, and analogous thereof with an identity of at least about 90%. In some embodiments, the first primer can have a sequence selected from any one of SEQ ID NOs. 380-758, and analogous thereof with an identity of at least about 98%. In some embodiments, the first primer can have a sequence selected from any one of SEQ ID NOs. 380-758.

Without intending to be bound by theory, it is believed that the novel features of the Alpha-RNA method herein include but are not limited to: i) a single primer near the gene exon-intron boundary is used to prime both genomic DNA and RNA (reversed transcribed into cDNA); ii) a novel method counting the quantities of genomic DNA and RNA is used; iii) a novel ratio based on RNA to DNA quantities of the same primer is developed to quantify RNA expression level per gene/cell, without the need in the conventional method that relies on house-keeping gene expression level, which is often variable across tissues and patients and associated with imprecise quantification.

Correspondingly, the invention's advantages include but are not limited to: i) high efficiency in quantifying clinically relevant genes; ii) high accuracy in measuring low-level but clinically important genes; iii) cost-efficient, as it allows target enrichment and sequencing of most clinically relevant genes; iv) broad applicability and can be used for various types of clinical samples, including the most accessible but low-quality clinical samples such as FFPE tumor tissues; v) high multiplexing ability (over 100s that is sufficient for clinical diagnosis); and vi) can be integrated with the main stream NGS workflow, without extra sample or additional processing.

Based on the next-generation sequencing (NGS) technology that is widely applicable and becoming the main workhorse in clinical diagnosis, it is believed that the Alpha-RNA method provided herein can open a new area for NGS to precisely and efficiently quantify RNA expressions of a set of highly clinical relevant genes. Alpha-RNA can be used to guide cancer immunotherapy, as the inventors have demonstrated in the clinical study on lung cancer patients who received checkpoint blockade immunotherapy.

An embodiment of the present invention relates to a method of identifying or determining a subject who could be responsive to an immune checkpoint inhibitor (i.e., a ICI responder), which includes the steps of obtaining a sample from the subject and then subjecting the sample to the method of measuring RNA expression level of a target sequence, as provided herein.

In an embodiment herein, the subject having high expression of CD274 and low expression of GZMA is identified or determined as responsive to the immune checkpoint inhibitor. In an embodiment herein, the subject having high expression of CD274 at single exon level is identified or determined as responsive to the immune checkpoint inhibitor. For example, the subject responsive to ICI may have high expression of CD274 exon6.

An embodiment of the present invention relates to a method of treating cancer, including applying an immune checkpoint inhibitor to a subject in need thereof, wherein the subject is tested for RNA expression level of a target sequence by using the method of of measuring RNA expression level of a target sequence as described herein before administrating the immune checkpoint inhibitor to the subject.

In an embodiment herein, the subject has high expression of CD274 and low expression of GZMA.

There is no gold standard/cutoff to determine high or low expression of target sequences. However, in this invention, in view of the reportedly about 20% NSLC patients response to ICIs^{3,4}, we define High and Low expression based on a cutoff percentile 20% among the patient group (>20% percentile as High expression and <=80% as Low expression). The cutoff percentile is calculated by using the RNA expression levels among all the NSCLC cancer patients.

In an embodiment herein, the subject has high expression of CD274 at single exon level. For example, the subject responsive to ICI may have high expression of CD274 exon6.

Without intending to be bound by theory, it is believed that the ability to pinpoint down to a single exon level, in contrast to what is generally available in conventional RNA-seq and expressed as RPM (read per million sequences), allows quantification of alternatively spliced mRNA variants that in certain scenario is important in disease initiation, progression, response to drugs, and patient survival.

The methods provided herein are applicable for cancers of any tissue type, of human and animal, and also any diseases, cancers and non-cancer diseases, where mRNA gene expression level testing are involved.

In some embodiments, the cancer is non-small cell lung cancer.

In an embodiment herein, the non-small cell lung cancer is selected from the group of adenocarcinoma, squamous cell carcinoma, large-cell lung carcinoma and a combination thereof. In an embodiment herein, the non-small cell lung cancer is selected from adenocarcinoma, squamous cell carcinoma and a combination thereof. In an embodiment herein, the non-small cell lung cancer can be squamous cell carcinoma.

In an embodiment herein, the immune checkpoint inhibitor is selected from the group of a PD-1 inhibitor, a PD-L1 inhibitor, and a combination thereof.

In an embodiment herein, the immune checkpoint inhibitor is selected from the group of pembrolizumab, nivolumab, atezolizumab, durvalumab, and a combination thereof.

An embodiment of the present invention relates to a kit for performing the above-described method of measuring RNA expression level of a target sequence in a sample by simultaneously enriching a target DNA and corresponding RNA and calculating a RNA-to-DNA ratio of the same target sequence, the kit includes:
(e) a reverse transcriptase for transcribing RNA into complementary DNA (cDNA);
(f) a universal sequencing adaptor to be ligated to a terminal of gDNA and cDNA in the sample;
(g) a first primer that binds to the cDNA and gDNA sequence at a position that is 3′ in relation to the target sequence on the cDNA or gDNA; and
(h) a second primer that binds to the target sequence at a position that is between the first primer and the target sequence on the cDNA or gDNA.

In some embodiments herein, the the first primer has a sequence selected from the group consisting of SEQ ID NOs. 1-379, and analogous thereof with an identity of at least about 80% of any one of SEQ ID NOs. 1-379. In some embodiments, the first primer can have a sequence selected from any one of SEQ ID NOs. 1-379, and analogous thereof with an identity of at least about 90%. In some embodiments, the first primer can have a sequence selected from any one of SEQ ID NOs. 1-379, and analogous thereof with an identity of at least about 98%. In some embodiments, the first primer can have a sequence selected from any one of SEQ ID NOs. 1-379.

In some embodiments herein, the second primer has a sequence selected from the group consisting of SEQ ID NOs. 380-758, and analogous thereof with an identity of at least about 80% of any one of SEQ ID NOs. 380-758. In some embodiments, the first primer can have a sequence selected from any one of SEQ ID NOs. 380-758, and analogous thereof with an identity of at least about 90%. In some embodiments, the first primer can have a sequence selected from any one of SEQ ID NOs. 380-758, and analogous thereof with an identity of at least about 98%. In some embodiments, the first primer can have a sequence selected from any one of SEQ ID NOs. 380-758.

Immune checkpoint inhibitors (ICIs) have shown unprecedented clinical activity in non-small cell lung cancer (NSCLC) and have dramatically changed how NSCLC is treated¹. Five ICIs have been approved for the treatment of NSCLC², including anti-PD-1 antibody pembrolizumab and nivolumab, anti-PD-L1 antibody atezolizumab and durvalumab, anti-CTLA antibody ipilimumab, which is required to be in combination with nivolumab. Although ICIs have durable response and prolonged survival in some patients, the objective response rate (ORR) is 20%-30%^{3,4} for unselected NSCLC patients.

Without intending to be bound by theory, it is believed that the development of next generation sequencing (NGS) provides comprehensive overview of molecular and genetic features of tumor profile, which may help ICI biomarker discovery. Several studies have found that structural variations of PD-L1 affect response to immunotherapy. One publication reported that 3′ region disruption of PD-L1 leads to its elevated expression, which enables immune evasion of EG7-OVA tumour cells in mice. This structural variation affects multiple cancers including adult T-cell leukaemia/lymphoma (ATL), diffuse large B cell lymphoma, and stomach adenocarcinoma¹⁶. Moreover, a few publications have reported the existence of secreted form of PD-L1(sPD-L1)^{17,18,19}. The function of sPD-L1 on the predictive of ICIs treatment is on debate. One research reported that C-terminal-deficient splicing variant of PD-L1 is highly secreted and this variant contribute to the resistance to anti-PD-L1 treatment in NSCLC¹⁷. Interestingly, another publication found that sPD-L1 concentration was elevated during ICIs but it did not affect the survival of NSCLC patients¹⁸. Thus, it is needed to investigate the structural variations of PD-L1 in NSCLC analyze whether structural variations of PD-L1 could serve as a prognostic marker for NSCLC patients.

The present invention develops a NGS-based method to detect the mRNA expression CD274 (encoding PD-L1) and its various splicing variants in NSCLC patients. The present invention finds that the mRNA expression detected by NGS has a good correlation with its protein expression detected by IHC (Dako 22C3). More importantly, survival analysis shows that high expression of CD274 exon3 and low expression of CD274 exon5, encoding the secreted and 3′-UTR truncated forms of PD-L1, has worst overall survival (OS) when compared with other splicing forms or low expression of PD-L1 among all NSCLC patients. These results suggest that detection of PD-L1 expression in NSCLC patients by next generation sequencing could be used to evaluate the prognosis of NSCLC patients and guide personal immune checkpoint therapy. Moreover, the examples herein analyze the NSCLC patients treated with anti-PD1 therapy and survival analysis show that patients with high expression of GZMA and CD274 have longer Progression-Free-Survival (PFS). Without intending to be bound by theory, it is believed that the personalized treatment based on the NGS method provided herein may improve PD-L1 therapy outcomes in non-small cell lung cancer (NSCLC).

Without intending to be bound by theory, it is believed that T cell infiltration is also of vital for cancer survival and ICI therapy¹⁰. T cell infiltration is an independent predictive marker for gastric cancer¹¹ and NSCLC¹², patients with higher CD8⁺ TIL density would have favorable survival. Also, NSCLC patients with increased CD8⁺ T cell infiltration may have longer PFS after ICI therapy¹³. However, patents could still fail from ICI therapy because of T cell exhaustion. It is believed that the exhausted T cells in the tumor microenvironment have decreased effector cytokine production and cytolytic activity, leading to the failure of cancer elimination¹⁴. One publication has shown that T cell exhaustion signatures could predict ICIs response¹⁵, patients with higher dysfunction score would have worse survival. Thus, the examples herein will also include cytotoxic marker and combine with PD-L1 to predict the survival of ICIs therapy.

### EXAMPLES

The examples illustrate a next-generation sequencing-based method to quantify mRNA expression of CD274 (encoding PD-L1), its splicing isoforms, T-cell activity factors, and other genetic drivers to triage patients for ICI therapies. The method uses anchored ligation priming for highly accurate RNA expression (Alpha-RNA). The examples evaluated Alpha-RNA with two retrospective non-small cell lung cancer (NSCLC) cohorts. Cohort I includes 182 NSCLC patients who underwent surgery resections, with available clinical IHC assay results and were followed up for overall survival analysis. Cohort II includes 33 patients who failed first-line therapies and further received anti-PD-1 therapies, prior to which needle biopsies were obtained, and were followed up for progression-free survival analysis following the treatment. In Cohort I, the mRNA expression of CD274 assessed by Alpha-RNA is highly correlated with routine clinical TIC protein expression (R²=0.51, P value <0.01). By Alpha-RNA, ALK mRNA is indeed expressed in those ALK fusion-positive cases and very low or no expression in ALK negative cases. Interestingly, patients with high expression of CD274 exon6 and low expression of exon4 (presumably resembling those secreted and 3′ UTR-truncated PD-L1 isoforms) had the worst overall survival when compared to patients with other CD274 splicing isoforms or patients with low CD274 expression (P value <0.01). The finding is consistent with the results from the analysis of the Cancer Genome Atlas (TCGA) data. Furthermore, it is found that patients with high expression of CD274 and low expression of GZMA have best response and progression-free survival to ICIs (P value <0.01), suggesting that GZMA, in additional to CD274, may be additional predictive marker for triaging patients to receive ICIs therapy. Overall, the data herein demonstrates that Alpha-RNA may be an effective molecular diagnosis assay for RNA expression of multiple genes and their isoforms to guide cancer immunotherapy.

### Materials and Methods

### Patients and Specimens

Cohort I retrospectively recruit 208 NSCLC patients who underwent surgical resection between 2014 and 2016 at Cancer Hospital of Zhejiang Province, Hangzhou, Zhejiang, China. Pathologic and clinicopathologic staging of the diagnoses are performed according to the World Health Organization (WHO) histological classification and tumor-node-metastasis (TNM) classification system. In total, 75 patients with squamous cell carcinoma and 131 patients with adenocarcinoma are included and basic clinical parameters are recorded. Written informed consent from all patients are obtained, and the study is approved by Research Ethics Committee of Cancer Hospital of Zhejiang Province (IRB-2018-24).

### Immunohistochemistry (IHC)

Lung tissues collected by surgery underwent fixation, dehydration, transparentizing, immersion and embedding followed by section at a thickness of 4 m. Epitope unmasking is done by antigen retrieval reagent. After washing with Tris Buffered Saline with Tween 20 (TBST), slides are incubated overnight at 4° C. with primary antibody. The expression of PD-L1 is then detected by detection kit following manufacturer's instruction. The anti-PD-L1 antibody is used to detect the protein level of PD-L1.

### Targeted DNA and RNA Sequencing

The example uses the anchored multiplex PCR method previously described for targeted DNA and RNA sequencing²⁰. First, total nucleic acid is extracted from formalin-fixed paraffin-embedded lung tissue samples with FormaPure XL RNA kit (Beckman Coulter life sciences, UK). Total nucleic acid is used to perform reverse transcription without removing genomic DNA. Briefly, random hexamer (300 ng/μL) and dNTP (10 nM) are added to sample DNA/RNA and incubated at 65° C. for 5 min to denature RNA. To synthesize first strand cDNA, SuperScript IV Reverse Transcriptase (Catalog no.18091050, Invitrogen), 5× buffer, RNaseOUT and fresh DTT are added and the following program is performed: 25° C. 10 min, 42° C. 30 min, 70° C. 15 min. Second strand cDNAs are then synthesized by incubating at 16° C. for 2 hours with the addition of DNA Polymerase I (Catalog no. M0209S, New England Biolabs), RNase H (Catalog no. M0297S, New England Biolabs), and 10× second strand reaction buffer (Catalog no. B6117S, New England Biolabs). The reverse transcription product is then purified with 1.8×AMPure XP magnetic beads (Catalog no. A63881, Beckman Coulter). Fragmented gDNA and cDNA mixture is ligated with an adaptor and followed by 15 cycles of PCR amplification. The amplicon products are purified and used for a hemi-nested PCR for enrichment of targets of interest. The example designs gene specific primers (GSPIs) and their 3′ nested primers GSP2 near the exon/intron boundary. At the presence of both genomic DNA and cDNA, a single primer near the exon boundary can prime and enrich both genomic DNA (containing exon and intron) and cDNA (spanning enamouring exons). Using this method, the RNA to DNA ratio per enrichment primer can be calculated and used to quantify RNA expression level.

### Statistical Analysis

Cox Proportional Hazards regression analyses are performed to assess the associations between biomarkers and overall survival. Differences are considered statistically significant at a two-sided p-value of <0.05. R studio, package survival (https://cran.r-project.org/web/packages/survival/index.html) and package survminer (https://cran.r-project.org/web/packages/survminer/index.html) are used to analyze the relationship between PD-L1 and patients' survival.

### Results

### Targeted Next Generation Sequencing and Immunohistochemistry

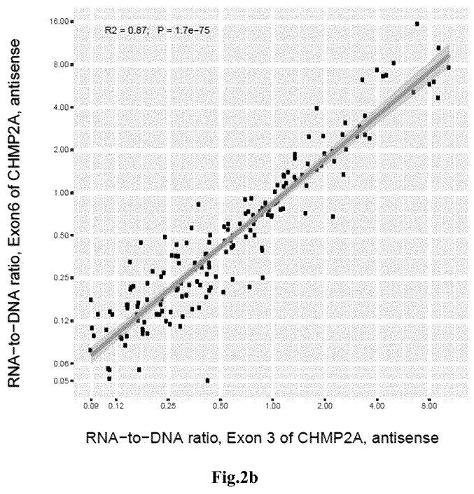

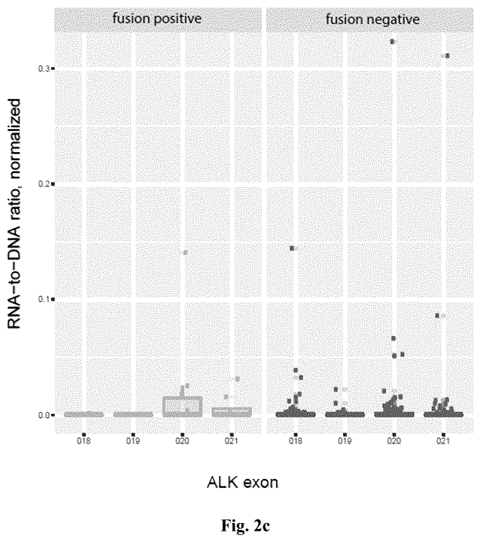

ALPHA-RNA method is based on anchored multiplex PCR, FIG. 1a and FIG. 1b show the schematic description of the targeted gDNA and cDNA sequencing. To test the reliability of the NGS method, RNA-to-DNA ratio of two house-keeping gene CHMP2A and B2M is calculated with antisense and sense. As shown in FIG. 2a, antisense RNA-to-DNA ratio has excellent correlation with sense RNA-to-DNA ratio both in CHMP2A and B2M, the correlation coefficient is 0.81 (p<0.001) and 0.86 (p<0.001) respectively. In the same house-keeping gene, RNA-to-DNA ratio of different exon is calculated, and RNA-to-DNA ratio of CHMP2A exon3 has good correlation with exon6 (FIG. 2b). ALK gene fusion and ALK expression are also quantified. As the box plot shows in FIG. 2c, ALK gene fusion leads to the up-regulation of ALK, which is consistent with other publications. IHC is the current standard method for PD-L1 detection but its limitation is obvious. Using the new method of this invention, the inventors quantify the expression of CD274 exons 3, and 5 and PDCD1 (encoding PD-1) exons 2 and 3. Also, protein expression of PD-L1 is assayed by IHC. The results showed that RNA expression of CD274 exons 3 and 5 are correlated with protein expression level of PD-L1 (correlation coefficient is 0.27 and 0.40, respectively, Spearman's rank test, FIG. 2d). The RNA expression of PD-1 (exons 2 and 3) is not correlated with PD-L1 protein expression (FIG. 2d).

### High Expression of CD274 Exon3 and Low Expression of CD274 Exon5 as a Negative Prognostic Marker in NSCLC Patients

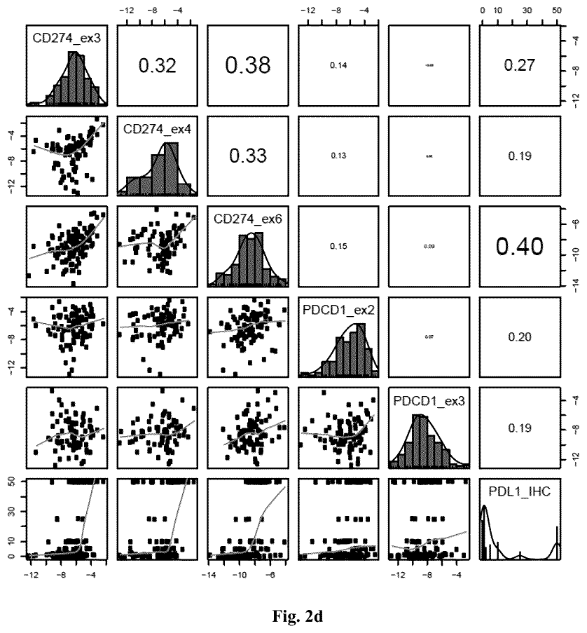

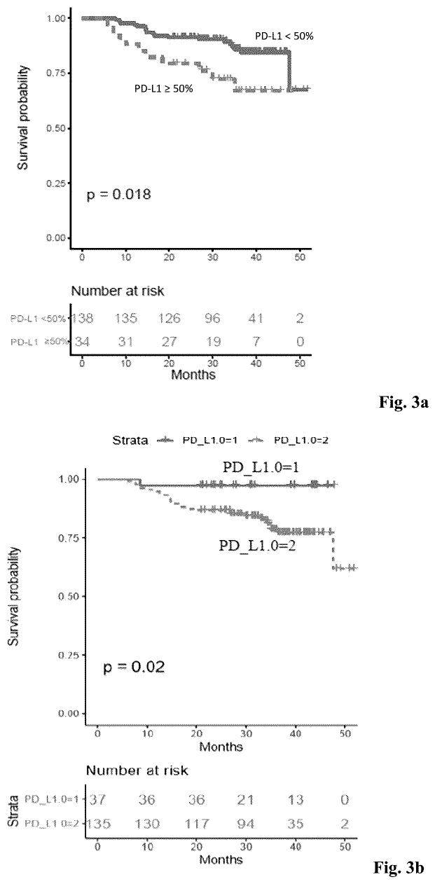

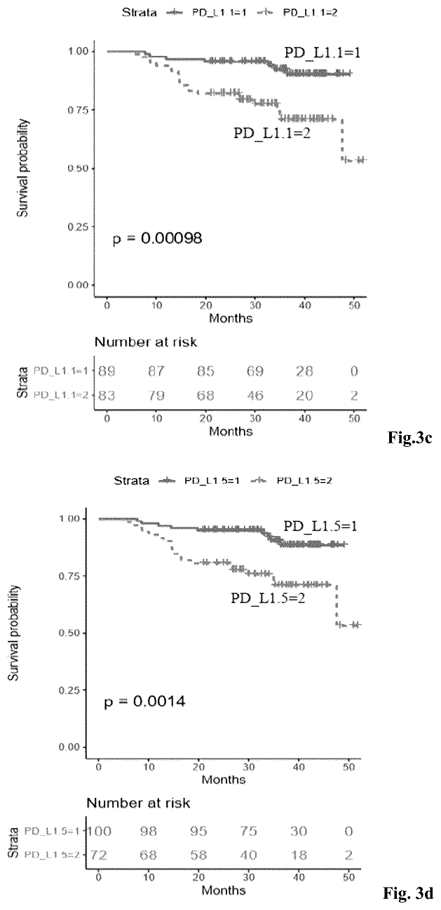

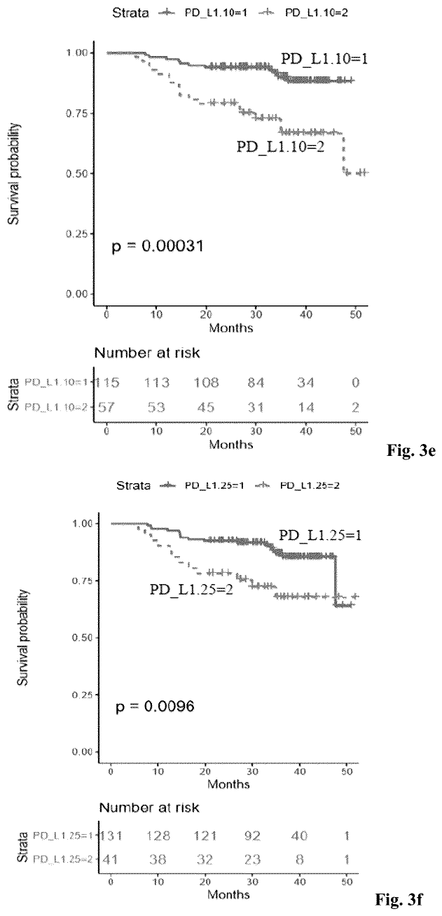

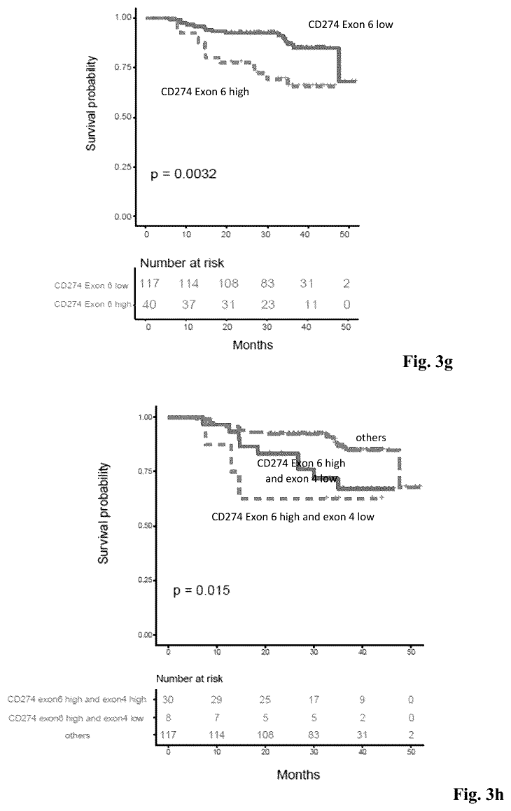

Next, the inventors assess the association between PD-L1 expression (both protein and RNA expression) and patient overall survival. Using different PD-L1 IHC positive tumor cellularity percentage cutoffs, patients with high expression of PD-L1 have shorter OS than patients with low PD-L1 expression consistently across the different cutoffs (FIG. 3a and FIGS. 3b-3f). Then, the inventors investigate whether RNA expression of CD274, at the exon-level/isoform level, could be used to predict NSCLC patients' survival. FIG. 3g shows that patients with high expression of CD274 exon6 have better OS. It is also found that patients with high expression of CD274 exon 6 and low expression of exon 4, likely reflecting the isoform with missing transmembrane domain, have the worst OS than patients with other expressions (P=0.00028, FIG. 3h). Univariate Cox regression analysis is performed and reveals that not only PD-L1 protein level but also the new PD-L1 RNA group (CD274 exon4− and exon6+) is significantly associated with survival (Table 1 below).

TABLE 1
Hazard ratios and 95% confidence intervals of PD-L1 protein expression
(at different cut-off percentiles, IHC assay) and PDL1 RNA expression
(continuous mean or dichotomous groups, ALPHA RNA assay) in studied
non-small cell lung cancer patients, with adjustment for gender,
age, and smoking status, using COX regression model.

| | |
|---|---|
| HR (95% CI for HR) | p. value |

| | | | |
|---|---|---|---|
| Gender | 0.49 | (0.2153-1.1363) | 0.097 |
| Age | 1 | (0.97309-1.0641) | 0.44 |
| Smoking | 1.6 | (0.7305-3.2984) | 0.25 |
| PD_L1.50 | 2.9 | (1.3143-6.2719) | 0.0082 |
| PD_L1.25 | 2.5 | (1.1851-5.4062) | 0.016 |
| PD_L1.10 | 3.6 | (1.6821-7.726) | 0.00098 |
| PD_L1.1 | 3.8 | (1.6002-8.8874) | 0.0024 |
| PDL1 RNA mean value | 2.2 | (1.023-4.761) | 0.044 |
| PDL1.RNA Group | 2.1 | (1.2495-3.6089) | 0.0054 |

### Validate the Prognostic Role of CD274 Using the Cancer Genome Atlas (TCGA) Data

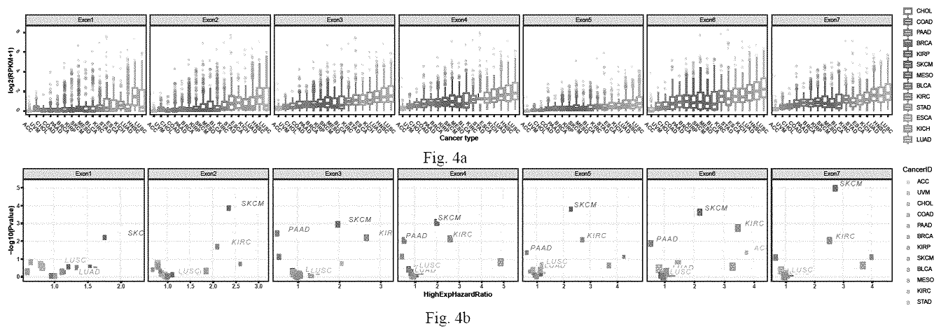

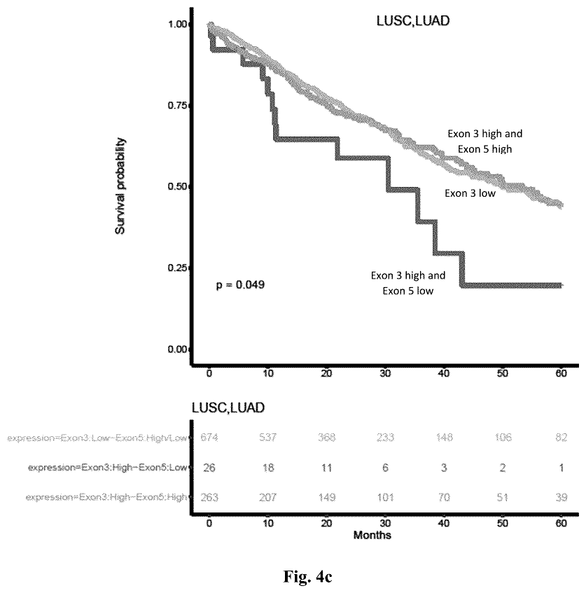

There is little data on the mRNA level of PD-L1 and NSCLC clinical outcomes. Thus, the inventors analyze the TCGA data across different tumor types and found that high expression of CD274 at the single exon level does not increase the hazard ratio (FIGS. 4a and 4b). However, in combined exon analysis, high expression of CD274 exon3 and low expression of CD274 exon5—the group likely resembling the secreted and 3′-UTR truncated forms of PD-L1—have the worst OS when compared with other splicing forms among all NSCLC patients (FIG. 4c).

### Investigate the Predictive Role of GZMA, PRF1, and CD274 in Immune Checkpoint Blockade Therapy in NSCLC Patients

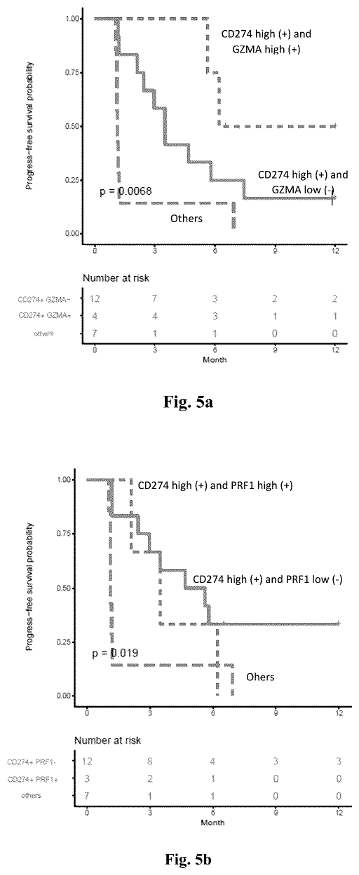

The success of anti-PD1/anti-PD-L1 therapy depends largely on the immune state of an individual. Thus, the inventors wonder whether the status of CD8⁺ T cells and the expression of its suppression ligand PD-L1 could predict the NSCLC patient response to anti-PD-1/PD-L1 therapies. This example have included 38 patients who have received the therapies and have available biopsies taken prior to the treatments, and with follow-up on treatment outcomes (stable disease [SD], partial response [PR] and complete response [PD]). We will use the method described before to quantify the expression of granzyme A, perforin, PD1 and PD-L1 (with isoforms). The expression of these genes is normalized by housekeeping genes (CHAMP and B2M). Progression Free Survival (PFS) is analyzed by CD8 signature genes and CD274 to investigate the predictive role of GZMA, PRF1, and CD274 isoforms in anti-PD-1/PD-L1 treatment outcomes in NSCLC patients. Survival analysis shows that GZA and CD274 double positive patients have the best PFS when they are treated with ICIs (FIG. 5a), but another CD8 signature gene PRF1 combined with CD274 does not improve PFS (FIG. 5b).

### Discussion

Without intending to be bound by theory, it is believed that PD-L1 is broadly expressed in NSCLC patients, both in adenocarcinoma and squamous cell carcinoma. The expression of inhibitory molecule PD-L1 and high infiltration of T cells in tumor microenvironment indicate that those exhausted T cells may be the reason for tumor escaping. Immune checkpoint inhibitors against PD-1/PD-L1 axis has produced huge clinical benefit, but the method to discriminate ICIs-responders and non-responders is poorly developed.

The present invention identifies one novel transcript of PD-L1, which has high expression of exon6 and low expression of exon4. One publication has reported highly secreted PD-L1 in Adult T-cell leukemia (ATL) patient, about 27% ATL has this sPD-L1 variant. In the examples herein, eight of 155 NSCLC patients have ex6+ ex4− transcript, which is consistent with previous publication. Patients with ex6+ ex4− transcript have poor survival than others, but univariate and multivariate. Cox regression shows that the novel transcript of this invention does not increase hazard (HR>1, but p value >0.05), more samples are needed to elucidate the function of ex6+ ex4− transcript.

To validate the predictive function of PD-L1 and other immune factors, the inventors use cohort II which contains 33 patients who fails first-line therapies and further receives anti-PD-1 therapies, prior to which needle biopsies are obtained. The result proves that patients with high expression of CD274 and low expression of GZMA have the best response to ICIs, which suggests that combination of CD274 and GZMA has the potential to be predictive marker for ICIs therapy.

Although, responders could be identified with this method, it is believed that some patients could still develop resistance to ICIs. Many genetic and epigenetic mechanisms enable the acquired resistance to ICIs therapy²¹. Downregulation of HLA class I molecules and loss of (B2M) expression have been described in melanoma patients treated with PD-1 blockade²² The loss of B2M results in impaired cell surface expression of MHC class I and defective antigen presentation, which mediates the resistance to ICI therapy. With the method according to the invention, one could adjust the primer panel and monitor the immune status of patients and combine ICI and chemotherapy, which could release more tumor antigens and generate adequate anti-tumor T cells. Despite this, it is believed that expression of alternative co-inhibitory immune checkpoints (e.g., CTLA-4, TIM-3, LAG-3, and VISTA) has been associated with resistance to ICIs²³, thus to improve the method, primer panels need to be enlarged.
1. Gandhi L, Rodriguez-Abreu D, Gadgeel S, et al. Pembrolizumab plus Chemotherapy in Metastatic Non-Small-Cell Lung Cancer. N Engl J Med. 2018; 378(22):2078-2092. doi:10.1056/NEJMoa1801005
2. Chiang A C, Herbst R S. Frontline immunotherapy for NSCLC the tale of the tail. Nat Rev Clin Oncol. 2020; 17(2):73-74. doi:10.1038/s41571-019-0317-y
3. Garon E B, Rizvi N A, Hui R, et al. Pembrolizumab for the Treatment of Non-Small-Cell Lung Cancer. N Engl J Med. 2015; 372(21):2018-2028. doi:10.1056/nejmoa1501824
4. Gettinger S N, Shepherd F A, Antonia S J, et al. First-line nivolumab (anti-PD-1; BMS-936558, ONO-4538) monotherapy in advanced NSCLC: Safety, efficacy, and correlation of outcomes with PD-L1 status. J Clin Oncol. 2014; 32(15_suppl):8024-8024. doi:10.1200/jco.2014.32.15_suppl.8024
5. Bodor J N, Boumber Y, Borghaei H. Biomarkers for immune checkpoint inhibition in non-small cell lung cancer (NSCLC). Cancer. 2020; 126(2):260-270. doi:10.1002/cncr.32468
6. Mok TSK, Wu Y-L, Kudaba I, et al. Pembrolizumab versus chemotherapy for previously untreated, PD-L1-expressing, locally advanced or metastatic non-small-cell lung cancer (KEYNOTE-042): a randomised, open-label, controlled, phase 3 trial. Lancet. 2019; 393(10183):1819-1830. doi:10.1016/S0140-6736(18)32409-7
7. Kintsler S, Cassataro M A, Drosch M, Holenya P, Knuechel R, Braunschweig T. Expression of programmed death ligand (PD-L1) in different tumors. Comparison of several current available antibody clones and antibody profiling. Ann Diagn Pathol. 2019; 41:24-37. doi:10.1016/J.ANNDIAGPATH.2019.05.005
8. Hirsch F R, McElhinny A, Stanforth D, et al. PD-L1 Immunohistochemistry Assays for Lung Cancer: Results from Phase 1 of the Blueprint PD-L1 IHC Assay Comparison Project. J Thorac Oncol. 2017; 12(2):208-222. doi:10.1016/J.JTHO.2016.11.2228
9. Lawson N L, Dix C I, Scorer P W, et al. Mapping the binding sites of antibodies utilized in programmed cell death ligand-1 predictive immunohistochemical assays for use with immuno-oncology therapies. Mod Pathol. 2020; 33(4):518-530. doi:10.1038/s41379-019-0372-z
10. Jiang Y, Li Y, Zhu B. T-cell exhaustion in the tumor microenvironment. Cell Death Dis. 2015; 6(6):e1792-e1792. doi:10.1038/cddis.2015.162
11. Zhang D, He W, Wu C, et al. Scoring system for tumor-infiltrating lymphocytes and its prognostic value for gastric cancer. Front Immunol. 2019; 10(JAN):71. doi:10.3389/fimmu.2019.00071
12. Tokito T, Azuma K, Kawahara A, et al. Predictive relevance of PD-L1 expression combined with CD8+ TIL density in stage III non-small cell lung cancer patients receiving concurrent chemoradiotherapy. Eur J Cancer. 2016; 55:7-14. doi:10.1016/j.ejca.2015.11.020
13. Fumet J-D, Richard C, Ledys F, et al. Prognostic and predictive role of CD8 and PD-L1 determination in lung tumor tissue of patients under anti-PD-1 therapy. Br J Cancer. 2018; 119(8):950-960. doi:10.1038/s41416-018-0220-9
14. Blank C U, Haining W N, Held W, et al. Defining ‘T cell exhaustion.’ Nat Rev Immunol. 2019; 19(11):665-674. doi:10.1038/s41577-019-0221-9
15. Wucherpfennig K W, Shirley Liu X, Jiang P, et al. Signatures of T cell dysfunction and exclusion predict cancer immunotherapy response. Nat Med. doi:10.1038/s41591-018-0136-1
16. Kataoka K, Shiraishi Y, Takeda Y, et al. Aberrant PD-L1 expression through 3′-UTR disruption in multiple cancers. Nature. 2016; 534(7607):402-406. doi:10.1038/nature18294
17. Gong B, Kiyotani K, Sakata S, et al. Secreted PD-L1 variants mediate resistance to PD-L1 blockade therapy in non-small cell lung cancer. J Exp Med. 2019; 216(4):982-1000. doi:10.1084/jem.20180870
18. Castello A, Rossi S, Toschi L, Mansi L, Lopci E. Soluble PD-L1 in NSCLC patients treated with checkpoint inhibitors and its correlation with metabolic parameters. Cancers (Basel). 2020; 12(6):1373. doi:10.3390/cancers12061373
19. Meyo M T, Jouinot A, Giroux-Leprieur E, et al. Predictive value of soluble PD-1, PD-L1, VEGFA, CD40 ligand and CD44 for nivolumab therapy in advanced non-small cell lung cancer: A case-control study. Cancers (Basel). 2020; 12(2):473. doi:10.3390/cancers12020473
20. Zheng Z, Liebers M, Zhelyazkova B, et al. Anchored multiplex PCR for targeted next-generation sequencing. Nat Med. 2014; 20(12):1479-1484. doi:10.1038/nm.3729
21. Jenkins R W, Barbie D A, Flaherty K T. Mechanisms of resistance to immune checkpoint inhibitors. Br J Cancer. 2018; 118(1):9-16. doi:10.1038/bjc.2017.434
22. Zaretsky J M, Garcia-Diaz A, Shin D S, et al. Mutations Associated with Acquired Resistance to PD-1 Blockade in Melanoma. N Engl J Med. 2016; 375(9):819-829. doi:10.1056/nejmoa1604958
23. Thommen D S, Schreiner J, Müller P, et al. Progression of lung cancer is associated with increased dysfunction of T cells defined by coexpression of multiple inhibitory receptors. Cancer Immunol Res. 2015; 3(12):1344-1354. doi:10.1158/2326-6066.CIR-15-0097

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A method of measuring RNA expression level of a target sequence in a sample by simultaneously enriching the target sequence and corresponding RNA and calculating a RNA-to-DNA ratio of the same target sequence, comprising the steps of:
(a) reverse transcribing RNA into complementary DNA (cDNA) resulting in a mixture of cDNA and genomic DNA (gDNA);
(b) ligating a universal sequencing adaptor to a terminal of the gDNA and cDNA from step (a);
(c) conducting a first polymerase chain reaction process to produce a single-stranded primer extension product comprising a complementary sequence, wherein the complementary sequence is complementary to the target sequence on the gDNA, the cDNA, or a combination thereof, and is complementary to the universal sequencing adaptor;
(d) conducting a second polymerase chain reaction process to amplify the single-stranded primer extension product; and
(e) performing a next-generation sequencing process and calculating the relative ratio of cDNA sequencing reads to gDNA sequencing reads, namely the RNA-to-DNA ratio, of the same target sequence,
wherein the target sequence is selected from a group consisting of a gene sequence, an exon of a gene, and a combination thereof.

2. The method of claim 1, wherein the first polymerase chain reaction process comprises the steps of:
(i) contacting a first primer that binds to the cDNA and gDNA sequence at a position that is 3′ in relation to the target sequence on the cDNA or gDNA;
(ii) extending by polymerization the first primer till the terminal comprising the ligated universal sequencing adaptor to produce the single-stranded primer extension product;
(iii) dissociating the single-stranded primer extension product comprising the complementary sequence to the target sequence from the template strand cDNA or gDNA; and
(iv) optionally, repeating steps (i) through (iii) one or more times.

3. The method of claim 2, wherein the second polymerase chain reaction process comprises binding a second primer to the target sequence at a position that is between the first primer and the target sequence on the cDNA or gDNA.

4. The method of claim 2, wherein steps (i) through (iii) are repeated for from about 1 cycles to about 100 cycles.

5. The method of claim 3, wherein the second primer is used to prime and enrich both gDNA and cDNA.

6. The method of claim 1, wherein the sample is selected from the group consisting of a formalin-fix paraffin-embedded tissue, fresh tissue collected by surgical biopsy or needle aspiration, blood, urine, ascites, pleural effusion, cerebrospinal fluid, pancreas cyst fluid, and a combination thereof.

7. The method of claim 1, wherein the method is for quantifying the expression of the target sequence selected from the group consisting of genes related to immune microenvironment, oncogenes, tumor suppressor genes, housekeeping genes, and other disease relevant genes.

8. The method of claim 1, wherein total nucleic acid is used to perform the reverse transcription in step (a) without removing genomic DNA.

9. The method of claim 1, wherein the first primer has a sequence selected from the group consisting of SEQ ID NOs. 1-379, and analogous thereof with an identity of at least about 80% of any one of SEQ ID NOs. 1-379.

10. The method of claim 3, wherein the second primer has a sequence selected from the group consisting of SEQ ID NOs. 380-758, and analogous thereof with an identity of at least about 80% of any one of SEQ ID NOs. 380-758.

11. A method of treating cancer in a subject comprising the steps of:
a. testing the subject for RNA expression level of a target sequence according to the method of claim 1; and
b. applying an immune checkpoint inhibitor to the subject.

12. The method of claim 11, wherein the subject has high expression of CD274 and low expression of GZMA.

13. The method of claim 11, wherein the subject has high expression of CD274 at single exon level.

14. The method of claim 11, wherein the cancer is non-small cell lung cancer.

15. The method of claim 14, wherein the non-small cell lung cancer is selected from the group consisting of adenocarcinoma, squamous cell carcinoma, large-cell lung carcinoma and a combination thereof.

16. The method of claim 11, wherein the immune checkpoint inhibitor is selected from the group consisting of a PD-1 inhibitor, a PD-L1 inhibitor, and a combination thereof.

17. The method of claim 11, wherein the immune checkpoint inhibitor is selected from the group consisting of pembrolizumab, nivolumab, atezolizumab, durvalumab, and a combination thereof.

18. A kit for performing the method of measuring RNA expression level of a target sequence in a sample by simultaneously enriching a target DNA and corresponding RNA and calculating a RNA-to-DNA ratio of the same target sequence according to claim 1, the kit comprising:
(a) a reverse transcriptase for transcribing RNA into complementary DNA (cDNA);
(b) a universal sequencing adaptor to be ligated to a terminal of gDNA and cDNA in the sample;
(c) a first primer that binds to the cDNA and gDNA sequence at a position that is 3′ in relation to the target sequence on the cDNA or gDNA; and
(d) a second primer that binds to the target sequence at a position that is between the first primer and the target sequence on the cDNA or gDNA.

19. The kit of claim 18, wherein the the first primer has a sequence selected from the group consisting of SEQ ID NOs. 1-379, and analogous thereof with an identity of at least about 80% of any one of SEQ ID NOs. 1-379.

20. The kit of claim 18, wherein the second primer has a sequence selected from the group consisting of SEQ ID NOs. 380-758, and analogous thereof with an identity of at least about 80% of any one of SEQ ID NOs. 380-758.


SEQUENCE LISTING (758 sequences)

SEQ ID NO: 1 - DNA, 44 bp, synthetic construct
  source 1..44: mol_type = other DNA
ttagggataacagggtaatcagatatcgatctgttagaaacctc

SEQ ID NO: 2 - DNA, 46 bp, synthetic construct
  source 1..46: mol_type = other DNA
ttagggataacagggtaatcagctgctgaaaatgtaactttgtatc

SEQ ID NO: 3 - DNA, 42 bp, synthetic construct
  source 1..42: mol_type = other DNA
ttagggataacagggtaatcggccgttgtacactcatcttcc

SEQ ID NO: 4 - DNA, 41 bp, synthetic construct
  source 1..41: mol_type = other DNA
ttagggataacagggtaatccacaaaatggatccagacaac

SEQ ID NO: 5 - DNA, 42 bp, synthetic construct
  source 1..42: mol_type = other DNA
ttagggataacagggtaatctaatgcttgctctgataggaaa

SEQ ID NO: 6 - DNA, 41 bp, synthetic construct
  source 1..41: mol_type = other DNA
ttagggataacagggtaatcttacacccagtggagaagctc

SEQ ID NO: 7 - DNA, 40 bp, synthetic construct
  source 1..40: mol_type = other DNA
ttagggataacagggtaatccccaccagaccatgagaggc

SEQ ID NO: 8 - DNA, 42 bp, synthetic construct
  source 1..42: mol_type = other DNA
ttagggataacagggtaatccagttaacgtcttccttctctc

SEQ ID NO: 9 - DNA, 40 bp, synthetic construct
  source 1..40: mol_type = other DNA
ttagggataacagggtaatcggacccccacacagcaaagc

SEQ ID NO: 10 - DNA, 40 bp, synthetic construct
  source 1..40: mol_type = other DNA
ttagggataacagggtaatcccctccttctggccaccatg

SEQ ID NO: 11 - DNA, 41 bp, synthetic construct
  source 1..41: mol_type = other DNA
ttagggataacagggtaatcctgggagccaatattgtcttt

SEQ ID NO: 12 - DNA, 41 bp, synthetic construct
  source 1..41: mol_type = other DNA
ttagggataacagggtaatcgaacgtactggtgaaaacacc

SEQ ID NO: 13 - DNA, 40 bp, synthetic construct
  source 1..40: mol_type = other DNA
ttagggataacagggtaatctcaggaaaatgctggctgac

SEQ ID NO: 14 - DNA, 40 bp, synthetic construct
  source 1..40: mol_type = other DNA
ttagggataacagggtaatcggtatgtggctacatgttcc

SEQ ID NO: 15 - DNA, 41 bp, synthetic construct
  source 1..41: mol_type = other DNA
ttagggataacagggtaatcctgaggaaggataggacaggg

SEQ ID NO: 16 - DNA, 42 bp, synthetic construct
  source 1..42: mol_type = other DNA
ttagggataacagggtaatccccataccctctcagcgtaccc

SEQ ID NO: 17 - DNA, 41 bp, synthetic construct
  source 1..41: mol_type = other DNA
ttagggataacagggtaatcataggggtttgctccggagag

SEQ ID NO: 18 - DNA, 41 bp, synthetic construct
  source 1..41: mol_type = other DNA
ttagggataacagggtaatctgttggatcatattcgtccac

SEQ ID NO: 19 - DNA, 45 bp, synthetic construct
  source 1..45: mol_type = other DNA
ttagggataacagggtaatctttattataaggcctgctgaaaatg

SEQ ID NO: 20 - DNA, 40 bp, synthetic construct
  source 1..40: mol_type = other DNA
ttagggataacagggtaatcaaagaaagccctccccagtc

SEQ ID NO: 21 - DNA, 44 bp, synthetic construct
  source 1..44: mol_type = other DNA
ttagggataacagggtaatccctacaggaagcaagtagtaattg

SEQ ID NO: 22 - DNA, 40 bp, synthetic construct
  source 1..40: mol_type = other DNA
ttagggataacagggtaatctgagttctgggcactgggtc

SEQ ID NO: 23 - DNA, 41 bp, synthetic construct
  source 1..41: mol_type = other DNA
ttagggataacagggtaatccacaacccactgaggtatatg

SEQ ID NO: 24 - DNA, 42 bp, synthetic construct
  source 1..42: mol_type = other DNA
ttagggataacagggtaatctaaatgttacgcagtgctaacc

SEQ ID NO: 25 - DNA, 44 bp, synthetic construct
  source 1..44: mol_type = other DNA
ttagggataacagggtaatcgttgcaaaccacaaaagtatactc

SEQ ID NO: 26 - DNA, 41 bp, synthetic construct
  source 1..41: mol_type = other DNA
ttagggataacagggtaatcgtcctttctgtaggctggatg

SEQ ID NO: 27 - DNA, 42 bp, synthetic construct
  source 1..42: mol_type = other DNA
ttagggataacagggtaatccatctgacttggtggtaaactt

SEQ ID NO: 28 - DNA, 42 bp, synthetic construct
  source 1..42: mol_type = other DNA
ttagggataacagggtaatcggaaaatgacaaagaacagctc

SEQ ID NO: 29 - DNA, 41 bp, synthetic construct
  source 1..41: mol_type = other DNA
ttagggataacagggtaatcccattttagcacttacctgtg

SEQ ID NO: 30 - DNA, 42 bp, synthetic construct
  source 1..42: mol_type = other DNA
ttagggataacagggtaatctgacattgcatacattcgaaag

SEQ ID NO: 31 - DNA, 46 bp, synthetic construct
  source 1..46: mol_type = other DNA
ttagggataacagggtaatcattatgaaaccaatattatggatccc

SEQ ID NO: 32 - DNA, 41 bp, synthetic construct
  source 1..41: mol_type = other DNA
ttagggataacagggtaatcgactcctgcttctgtcatatc

SEQ ID NO: 33 - DNA, 40 bp, synthetic construct
  source 1..40: mol_type = other DNA
ttagggataacagggtaatcgagaggatcagcgagagtgg

SEQ ID NO: 34 - DNA, 40 bp, synthetic construct
  source 1..40: mol_type = other DNA
ttagggataacagggtaatcacctggccttcatacacctc

SEQ ID NO: 35 - DNA, 40 bp, synthetic construct
  source 1..40: mol_type = other DNA
ttagggataacagggtaatcgtcagtggggctgcggcatg

SEQ ID NO: 36 - DNA, 43 bp, synthetic construct
  source 1..43: mol_type = other DNA
ttagggataacagggtaatccaatttcttgcagaacacacagc

SEQ ID NO: 37 - DNA, 40 bp, synthetic construct
  source 1..40: mol_type = other DNA
ttagggataacagggtaatcttttccaaggacggttgaag

SEQ ID NO: 38 - DNA, 40 bp, synthetic construct
  source 1..40: mol_type = other DNA
ttagggataacagggtaatccgaaggactagtttggaacc

SEQ ID NO: 39 - DNA, 42 bp, synthetic construct
  source 1..42: mol_type = other DNA
ttagggataacagggtaatcacatatactctccagaatcagc

SEQ ID NO: 40 - DNA, 41 bp, synthetic construct
  source 1..41: mol_type = other DNA
ttagggataacagggtaatctgaagacacatatgctccttc

SEQ ID NO: 41 - DNA, 40 bp, synthetic construct
  source 1..40: mol_type = other DNA
ttagggataacagggtaatctccttctccgcacattttac

SEQ ID NO: 42 - DNA, 40 bp, synthetic construct
  source 1..40: mol_type = other DNA
ttagggataacagggtaatcgcatctccaccgccgtgtgc

SEQ ID NO: 43 - DNA, 41 bp, synthetic construct
  source 1..41: mol_type = other DNA
ttagggataacagggtaatctcttctccaccgggtctccag

SEQ ID NO: 44 - DNA, 42 bp, synthetic construct
  source 1..42: mol_type = other DNA
ttagggataacagggtaatcgcacaaggagcagcgtagaaag

SEQ ID NO: 45 - DNA, 40 bp, synthetic construct
  source 1..40: mol_type = other DNA
ttagggataacagggtaatcgggagctgccacccaatgtc

SEQ ID NO: 46 - DNA, 40 bp, synthetic construct
  source 1..40: mol_type = other DNA
ttagggataacagggtaatctctgaggttggagattcgag

SEQ ID NO: 47 - DNA, 41 bp, synthetic construct
  source 1..41: mol_type = other DNA
ttagggataacagggtaatcggtgtccccgatgtcattcgc

SEQ ID NO: 48 - DNA, 41 bp, synthetic construct
  source 1..41: mol_type = other DNA
ttagggataacagggtaatctcccaccacagacgcaatcac

SEQ ID NO: 49 - DNA, 43 bp, synthetic construct
  source 1..43: mol_type = other DNA
ttagggataacagggtaatcttgtcttgattttactttcccaa

SEQ ID NO: 50 - DNA, 40 bp, synthetic construct
  source 1..40: mol_type = other DNA
ttagggataacagggtaatctggcagagtcatcatcattg

SEQ ID NO: 51 - DNA, 40 bp, synthetic construct
  source 1..40: mol_type = other DNA
ttagggataacagggtaatcacaggcaaaagcagcaagtc

SEQ ID NO: 52 - DNA, 43 bp, synthetic construct
  source 1..43: mol_type = other DNA
ttagggataacagggtaatcgctggctgagtcctcctcaccac

SEQ ID NO: 53 - DNA, 40 bp, synthetic construct
  source 1..40: mol_type = other DNA
ttagggataacagggtaatctcagcacgatgtctctcctc

SEQ ID NO: 54 - DNA, 40 bp, synthetic construct
  source 1..40: mol_type = other DNA
ttagggataacagggtaatcggctccccaggctcgtgtcc

SEQ ID NO: 55 - DNA, 44 bp, synthetic construct
  source 1..44: mol_type = other DNA
ttagggataacagggtaatccttcaggacgttgaactctgacag

SEQ ID NO: 56 - DNA, 40 bp, synthetic construct
  source 1..40: mol_type = other DNA
ttagggataacagggtaatcaggaccttggctgcatgaag

SEQ ID NO: 57 - DNA, 41 bp, synthetic construct
  source 1..41: mol_type = other DNA
ttagggataacagggtaatctagtaatttgggaatgcctgg

SEQ ID NO: 58 - DNA, 44 bp, synthetic construct
  source 1..44: mol_type = other DNA
ttagggataacagggtaatcctgcaggatccattaaatgtcatc

SEQ ID NO: 59 - DNA, 41 bp, synthetic construct
  source 1..41: mol_type = other DNA
ttagggataacagggtaatcggcaggaagattttcaatctc

SEQ ID NO: 60 - DNA, 45 bp, synthetic construct
  source 1..45: mol_type = other DNA
ttagggataacagggtaatctcaagtttaatatcccatgattgtc

SEQ ID NO: 61 - DNA, 42 bp, synthetic construct
  source 1..42: mol_type = other DNA
ttagggataacagggtaatcatgtgacattcgtctacctcac

SEQ ID NO: 62 - DNA, 40 bp, synthetic construct
  source 1..40: mol_type = other DNA
ttagggataacagggtaatccgacgaggtggcctgtcgtc

SEQ ID NO: 63 - DNA, 41 bp, synthetic construct
  source 1..41: mol_type = other DNA
ttagggataacagggtaatctcctccatctcatagctgtcg

SEQ ID NO: 64 - DNA, 46 bp, synthetic construct
  source 1..46: mol_type = other DNA
ttagggataacagggtaatcgagtgagtctttaaattcaccaatac

SEQ ID NO: 65 - DNA, 41 bp, synthetic construct
  source 1..41: mol_type = other DNA
ttagggataacagggtaatcgcggggagaactggggtgctg

SEQ ID NO: 66 - DNA, 41 bp, synthetic construct
  source 1..41: mol_type = other DNA
ttagggataacagggtaatcatccaaatgaggagtgtgtac

SEQ ID NO: 67 - DNA, 45 bp, synthetic construct
  source 1..45: mol_type = other DNA
ttagggataacagggtaatcgtaatggactggatatatcagagtc

SEQ ID NO: 68 - DNA, 46 bp, synthetic construct
  source 1..46: mol_type = other DNA
ttagggataacagggtaatcactcctcgccggctctttttcaattg

SEQ ID NO: 69 - DNA, 40 bp, synthetic construct
  source 1..40: mol_type = other DNA
ttagggataacagggtaatccagcatggtgagcagctcag

SEQ ID NO: 70 - DNA, 40 bp, synthetic construct
  source 1..40: mol_type = other DNA
ttagggataacagggtaatccctggagccacatcctcccc

SEQ ID NO: 71 - DNA, 44 bp, synthetic construct
  source 1..44: mol_type = other DNA
ttagggataacagggtaatcccttttcagaacaatgttatgtcg

SEQ ID NO: 72 - DNA, 40 bp, synthetic construct
  source 1..40: mol_type = other DNA
ttagggataacagggtaatctccttgcctccactcacacc

SEQ ID NO: 73 - DNA, 41 bp, synthetic construct
  source 1..41: mol_type = other DNA
ttagggataacagggtaatctcctgagggcaaatgttgatg

SEQ ID NO: 74 - DNA, 41 bp, synthetic construct
  source 1..41: mol_type = other DNA
ttagggataacagggtaatctgggcaaacttgtggtagcag

SEQ ID NO: 75 - DNA, 41 bp, synthetic construct
  source 1..41: mol_type = other DNA
ttagggataacagggtaatctcttctcctggtctgtggaac

SEQ ID NO: 76 - DNA, 40 bp, synthetic construct
  source 1..40: mol_type = other DNA
ttagggataacagggtaatcgactctgtaggctgcagttc

SEQ ID NO: 77 - DNA, 41 bp, synthetic construct
  source 1..41: mol_type = other DNA
ttagggataacagggtaatcttgctccttccatccttgctc

SEQ ID NO: 78 - DNA, 40 bp, synthetic construct
  source 1..40: mol_type = other DNA
ttagggataacagggtaatcaagggacaagcagccacacc

SEQ ID NO: 79 - DNA, 44 bp, synthetic construct
  source 1..44: mol_type = other DNA
ttagggataacagggtaatccaaaagagaaatcacgcatttatg

SEQ ID NO: 80 - DNA, 41 bp, synthetic construct
  source 1..41: mol_type = other DNA
ttagggataacagggtaatcgctgtctgcatttatgaaccc

SEQ ID NO: 81 - DNA, 41 bp, synthetic construct
  source 1..41: mol_type = other DNA
ttagggataacagggtaatcacttgcccagcgtgtcctctc

SEQ ID NO: 82 - DNA, 41 bp, synthetic construct
  source 1..41: mol_type = other DNA
ttagggataacagggtaatccagttagagggcccacagagg

SEQ ID NO: 83 - DNA, 45 bp, synthetic construct
  source 1..45: mol_type = other DNA
ttagggataacagggtaatcgagacatgcatgaacatttttctcc

SEQ ID NO: 84 - DNA, 44 bp, synthetic construct
  source 1..44: mol_type = other DNA
ttagggataacagggtaatcttttgatctctgaatttgcaagag

SEQ ID NO: 85 - DNA, 40 bp, synthetic construct
  source 1..40: mol_type = other DNA
ttagggataacagggtaatcctggaaggcaggtaggatcc

SEQ ID NO: 86 - DNA, 40 bp, synthetic construct
  source 1..40: mol_type = other DNA
ttagggataacagggtaatcccccaaaccccaatgaagag

SEQ ID NO: 87 - DNA, 40 bp, synthetic construct
  source 1..40: mol_type = other DNA
ttagggataacagggtaatctttgcagaaaacagatctgt

SEQ ID NO: 88 - DNA, 40 bp, synthetic construct
  source 1..40: mol_type = other DNA
ttagggataacagggtaatcaagttgtggacaggttttga

SEQ ID NO: 89 - DNA, 40 bp, synthetic construct
  source 1..40: mol_type = other DNA
ttagggataacagggtaatctttcagttcaatgcatgctg

SEQ ID NO: 90 - DNA, 40 bp, synthetic construct
  source 1..40: mol_type = other DNA
ttagggataacagggtaatccggggccgcggaaaggaagg

SEQ ID NO: 91 - DNA, 40 bp, synthetic construct
  source 1..40: mol_type = other DNA
ttagggataacagggtaatccccacgtggcggagggactg

SEQ ID NO: 92 - DNA, 40 bp, synthetic construct
  source 1..40: mol_type = other DNA
ttagggataacagggtaatcctggccctcccactcctgac

SEQ ID NO: 93 - DNA, 40 bp, synthetic construct
  source 1..40: mol_type = other DNA
ttagggataacagggtaatcatcctggtcccctttcatgc

SEQ ID NO: 94 - DNA, 39 bp, synthetic construct
  source 1..39: mol_type = other DNA
ttagggataacagggtaatctgtactcagggctctgcag

SEQ ID NO: 95 - DNA, 40 bp, synthetic construct
  source 1..40: mol_type = other DNA
ttagggataacagggtaatcgccacccatatgtaccatcg

SEQ ID NO: 96 - DNA, 42 bp, synthetic construct
  source 1..42: mol_type = other DNA
ttagggataacagggtaatcattctccaaaatggcccgagac

SEQ ID NO: 97 - DNA, 40 bp, synthetic construct
  source 1..40: mol_type = other DNA
ttagggataacagggtaatcacgagtacctcatcccacag

SEQ ID NO: 98 - DNA, 40 bp, synthetic construct
  source 1..40: mol_type = other DNA
ttagggataacagggtaatcattttgaaggagacggactg

SEQ ID NO: 99 - DNA, 40 bp, synthetic construct
  source 1..40: mol_type = other DNA
ttagggataacagggtaatcccatcaccacgaaatccttg

SEQ ID NO: 100 - DNA, 41 bp, synthetic construct
  source 1..41: mol_type = other DNA
ttagggataacagggtaatcaggctttcacgttagttagtg

SEQ ID NO: 101 - DNA, 43 bp, synthetic construct
  source 1..43: mol_type = other DNA
ttagggataacagggtaatctctcccaatcatcactcgagtcc

SEQ ID NO: 102 - DNA, 40 bp, synthetic construct
  source 1..40: mol_type = other DNA
ttagggataacagggtaatctgaggtgtaggtgctgtcac

SEQ ID NO: 103 - DNA, 41 bp, synthetic construct
  source 1..41: mol_type = other DNA
ttagggataacagggtaatcaaaggtcagtgggattgtaac

SEQ ID NO: 104 - DNA, 40 bp, synthetic construct
  source 1..40: mol_type = other DNA
ttagggataacagggtaatctaagcactgtcaccccttcc

SEQ ID NO: 105 - DNA, 41 bp, synthetic construct
  source 1..41: mol_type = other DNA
ttagggataacagggtaatcgtcctcatgtattggtctctc

SEQ ID NO: 106 - DNA, 44 bp, synthetic construct
  source 1..44: mol_type = other DNA
ttagggataacagggtaatccagaaaacaagtggttatagatgg

SEQ ID NO: 107 - DNA, 40 bp, synthetic construct
  source 1..40: mol_type = other DNA
ttagggataacagggtaatcaagtggttctggattagctg

SEQ ID NO: 108 - DNA, 41 bp, synthetic construct
  source 1..41: mol_type = other DNA
ttagggataacagggtaatctggtgtgaaatgactgagtac

SEQ ID NO: 109 - DNA, 42 bp, synthetic construct
  source 1..42: mol_type = other DNA
ttagggataacagggtaatcgacaggatgacaggaagagcac

SEQ ID NO: 110 - DNA, 40 bp, synthetic construct
  source 1..40: mol_type = other DNA
ttagggataacagggtaatctcgtcagtggcccgcttctg

SEQ ID NO: 111 - DNA, 40 bp, synthetic construct
  source 1..40: mol_type = other DNA
ttagggataacagggtaatcgtcagcattactctgtgtcc

SEQ ID NO: 112 - DNA, 40 bp, synthetic construct
  source 1..40: mol_type = other DNA
ttagggataacagggtaatccaggccttcccctctctttc

SEQ ID NO: 113 - DNA, 43 bp, synthetic construct
  source 1..43: mol_type = other DNA
ttagggataacagggtaatccttgtttccgtgaaagactatac

SEQ ID NO: 114 - DNA, 43 bp, synthetic construct
  source 1..43: mol_type = other DNA
ttagggataacagggtaatcactagggctacttctttgatttc

SEQ ID NO: 115 - DNA, 44 bp, synthetic construct
  source 1..44: mol_type = other DNA
ttagggataacagggtaatccctattatgacttgtcacaatgtc

SEQ ID NO: 116 - DNA, 41 bp, synthetic construct
  source 1..41: mol_type = other DNA
ttagggataacagggtaatctctttttctgtttggcttgac

SEQ ID NO: 117 - DNA, 40 bp, synthetic construct
  source 1..40: mol_type = other DNA
ttagggataacagggtaatctttacccctccttcctagag

SEQ ID NO: 118 - DNA, 40 bp, synthetic construct
  source 1..40: mol_type = other DNA
ttagggataacagggtaatcccaccccaagagagcaacac

SEQ ID NO: 119 - DNA, 41 bp, synthetic construct
  source 1..41: mol_type = other DNA
ttagggataacagggtaatcgtgatgtccaccctgttcctg

SEQ ID NO: 120 - DNA, 40 bp, synthetic construct
  source 1..40: mol_type = other DNA
ttagggataacagggtaatcgcagaacaggatgcagagac

SEQ ID NO: 121 - DNA, 41 bp, synthetic construct
  source 1..41: mol_type = other DNA
ttagggataacagggtaatctggtggaaagaacctctcaac

SEQ ID NO: 122 - DNA, 41 bp, synthetic construct
  source 1..41: mol_type = other DNA
ttagggataacagggtaatcctctgagtagaaccattgctc

SEQ ID NO: 123 - DNA, 40 bp, synthetic construct
  source 1..40: mol_type = other DNA
ttagggataacagggtaatcgggtgggccgaagtctgacc

SEQ ID NO: 124 - DNA, 40 bp, synthetic construct
  source 1..40: mol_type = other DNA
ttagggataacagggtaatcgccttcggctccacctggtc

SEQ ID NO: 125 - DNA, 43 bp, synthetic construct
  source 1..43: mol_type = other DNA
ttagggataacagggtaatcgtatgtttggtagttgcatatgg

SEQ ID NO: 126 - DNA, 43 bp, synthetic construct
  source 1..43: mol_type = other DNA
ttagggataacagggtaatcaatatttcgtatggtgccatttg

SEQ ID NO: 127 - DNA, 41 bp, synthetic construct
  source 1..41: mol_type = other DNA
ttagggataacagggtaatcctcctgatgagaagagggttg

SEQ ID NO: 128 - DNA, 43 bp, synthetic construct
  source 1..43: mol_type = other DNA
ttagggataacagggtaatctcactgcagttgtaggttataac

SEQ ID NO: 129 - DNA, 46 bp, synthetic construct
  source 1..46: mol_type = other DNA
ttagggataacagggtaatcaaaatatttcaatgggtcatatcaca

SEQ ID NO: 130 - DNA, 41 bp, synthetic construct
  source 1..41: mol_type = other DNA
ttagggataacagggtaatctagtcactggcagcaacagtc

SEQ ID NO: 131 - DNA, 41 bp, synthetic construct
  source 1..41: mol_type = other DNA
ttagggataacagggtaatcttcctcaggattgcctttacc

SEQ ID NO: 132 - DNA, 40 bp, synthetic construct
  source 1..40: mol_type = other DNA
ttagggataacagggtaatcactgcattctgactttcagt

SEQ ID NO: 133 - DNA, 44 bp, synthetic construct
  source 1..44: mol_type = other DNA
ttagggataacagggtaatcaaaaagctctaattctctacatgc

SEQ ID NO: 134 - DNA, 42 bp, synthetic construct
  source 1..42: mol_type = other DNA
ttagggataacagggtaatcatgtgtcatcctgtaagctacc

SEQ ID NO: 135 - DNA, 40 bp, synthetic construct
  source 1..40: mol_type = other DNA
ttagggataacagggtaatcgtggcccctgagcgtcatct

SEQ ID NO: 136 - DNA, 40 bp, synthetic construct
  source 1..40: mol_type = other DNA
ttagggataacagggtaatcacccctagacccaaatcctc

SEQ ID NO: 137 - DNA, 40 bp, synthetic construct
  source 1..40: mol_type = other DNA
ttagggataacagggtaatccgctcccagtggtgcctgcg

SEQ ID NO: 138 - DNA, 40 bp, synthetic construct
  source 1..40: mol_type = other DNA
ttagggataacagggtaatcgctcagaacctggtatctac

SEQ ID NO: 139 - DNA, 40 bp, synthetic construct
  source 1..40: mol_type = other DNA
ttagggataacagggtaatcgctgggagcagcggggagag

SEQ ID NO: 140 - DNA, 41 bp, synthetic construct
  source 1..41: mol_type = other DNA
ttagggataacagggtaatcggcgtcctactggcatgaccc

SEQ ID NO: 141 - DNA, 40 bp, synthetic construct
  source 1..40: mol_type = other DNA
ttagggataacagggtaatctggaggacctggaccgtgtc

SEQ ID NO: 142 - DNA, 40 bp, synthetic construct
  source 1..40: mol_type = other DNA
ttagggataacagggtaatcggtgatcatcgaccaagtcc

SEQ ID NO: 143 - DNA, 43 bp, synthetic construct
  source 1..43: mol_type = other DNA
ttagggataacagggtaatctgagacattgttggccaaaatag

SEQ ID NO: 144 - DNA, 40 bp, synthetic construct
  source 1..40: mol_type = other DNA
ttagggataacagggtaatcactgcagcagccaccgtgag

SEQ ID NO: 145 - DNA, 41 bp, synthetic construct
  source 1..41: mol_type = other DNA
ttagggataacagggtaatcattgattcaatgaccctccag

SEQ ID NO: 146 - DNA, 44 bp, synthetic construct
  source 1..44: mol_type = other DNA
ttagggataacagggtaatctccataggctgttccttcaaccac

SEQ ID NO: 147 - DNA, 40 bp, synthetic construct
  source 1..40: mol_type = other DNA
ttagggataacagggtaatcttcagttcagacatgagagc

SEQ ID NO: 148 - DNA, 40 bp, synthetic construct
  source 1..40: mol_type = other DNA
ttagggataacagggtaatcagatggcttgatcctgagtc

SEQ ID NO: 149 - DNA, 40 bp, synthetic construct
  source 1..40: mol_type = other DNA
ttagggataacagggtaatcggatgagcctgaccagtgag

SEQ ID NO: 150 - DNA, 40 bp, synthetic construct
  source 1..40: mol_type = other DNA
ttagggataacagggtaatctccagcactctgacatatgg

SEQ ID NO: 151 - DNA, 41 bp, synthetic construct
  source 1..41: mol_type = other DNA
ttagggataacagggtaatccaagtgaattgcagtccttcc

SEQ ID NO: 152 - DNA, 44 bp, synthetic construct
  source 1..44: mol_type = other DNA
ttagggataacagggtaatcttctctctccagagtgctctaatg

SEQ ID NO: 153 - DNA, 40 bp, synthetic construct
  source 1..40: mol_type = other DNA
ttagggataacagggtaatcaaaaggtgacatggaaagcc

SEQ ID NO: 154 - DNA, 44 bp, synthetic construct
  source 1..44: mol_type = other DNA
ttagggataacagggtaatcatggtactgcatgcgcttgacatc

SEQ ID NO: 155 - DNA, 41 bp, synthetic construct
  source 1..41: mol_type = other DNA
ttagggataacagggtaatcagacaataaaaggcagcttgg

SEQ ID NO: 156 - DNA, 45 bp, synthetic construct
  source 1..45: mol_type = other DNA
ttagggataacagggtaatctgaatttaaatggttttcttttctc

SEQ ID NO: 157 - DNA, 42 bp, synthetic construct
  source 1..42: mol_type = other DNA
ttagggataacagggtaatctcctttgcaggactgtcaagca

SEQ ID NO: 158 - DNA, 41 bp, synthetic construct
  source 1..41: mol_type = other DNA
ttagggataacagggtaatctcatcaggagagcatttaagg

SEQ ID NO: 159 - DNA, 46 bp, synthetic construct
  source 1..46: mol_type = other DNA
ttagggataacagggtaatcaccataaaatcatatgctccactaac

SEQ ID NO: 160 - DNA, 43 bp, synthetic construct
  source 1..43: mol_type = other DNA
ttagggataacagggtaatccatgttttgatgggtcatgttgc

SEQ ID NO: 161 - DNA, 40 bp, synthetic construct
  source 1..40: mol_type = other DNA
ttagggataacagggtaatcgaagtcccaaccatgacaag

SEQ ID NO: 162 - DNA, 44 bp, synthetic construct
  source 1..44: mol_type = other DNA
ttagggataacagggtaatctcctttctacccaaaagtaatcat

SEQ ID NO: 163 - DNA, 44 bp, synthetic construct
  source 1..44: mol_type = other DNA
ttagggataacagggtaatcaagaggaagcttagatagttctcg

SEQ ID NO: 164 - DNA, 45 bp, synthetic construct
  source 1..45: mol_type = other DNA
ttagggataacagggtaatcgaatgtgtttctccatacaggtcac

SEQ ID NO: 165 - DNA, 41 bp, synthetic construct
  source 1..41: mol_type = other DNA
ttagggataacagggtaatcatctttgtcatcagctgaaga

SEQ ID NO: 166 - DNA, 40 bp, synthetic construct
  source 1..40: mol_type = other DNA
ttagggataacagggtaatcatctccctccaaaagtggtg

SEQ ID NO: 167 - DNA, 40 bp, synthetic construct
  source 1..40: mol_type = other DNA
ttagggataacagggtaatccagatagccctggacaaacc

SEQ ID NO: 168 - DNA, 40 bp, synthetic construct
  source 1..40: mol_type = other DNA
ttagggataacagggtaatcaggtgccctactacctggag

SEQ ID NO: 169 - DNA, 41 bp, synthetic construct
  source 1..41: mol_type = other DNA
ttagggataacagggtaatctggtcctgtgagggctgcaag

SEQ ID NO: 170 - DNA, 42 bp, synthetic construct
  source 1..42: mol_type = other DNA
ttagggataacagggtaatcggaagagctgccaggcctgccg

SEQ ID NO: 171 - DNA, 42 bp, synthetic construct
  source 1..42: mol_type = other DNA
ttagggataacagggtaatctttgtataaaagtttacacggg

SEQ ID NO: 172 - DNA, 40 bp, synthetic construct
  source 1..40: mol_type = other DNA
ttagggataacagggtaatcgggtgaagtggggtctgctg

SEQ ID NO: 173 - DNA, 41 bp, synthetic construct
  source 1..41: mol_type = other DNA
ttagggataacagggtaatcttagagcgtttgatcatgagc

SEQ ID NO: 174 - DNA, 40 bp, synthetic construct
  source 1..40: mol_type = other DNA
ttagggataacagggtaatcgatggtcagtgccttgttgg

SEQ ID NO: 175 - DNA, 40 bp, synthetic construct
  source 1..40: mol_type = other DNA
ttagggataacagggtaatctcactgaagggtctggtagg

SEQ ID NO: 176 - DNA, 41 bp, synthetic construct
  source 1..41: mol_type = other DNA
ttagggataacagggtaatcagggagctggttcacatgatc

SEQ ID NO: 177 - DNA, 42 bp, synthetic construct
  source 1..42: mol_type = other DNA
ttagggataacagggtaatctcacaacaagttcttgaaaagc

SEQ ID NO: 178 - DNA, 40 bp, synthetic construct
  source 1..40: mol_type = other DNA
ttagggataacagggtaatctctagggatttcagcactcc

SEQ ID NO: 179 - DNA, 40 bp, synthetic construct
  source 1..40: mol_type = other DNA
ttagggataacagggtaatccagcagggattatctgaacc

SEQ ID NO: 180 - DNA, 44 bp, synthetic construct
  source 1..44: mol_type = other DNA
ttagggataacagggtaatcagttagtatatacaatgccacctg

SEQ ID NO: 181 - DNA, 41 bp, synthetic construct
  source 1..41: mol_type = other DNA
ttagggataacagggtaatctagtctactttgtccccagtc

SEQ ID NO: 182 - DNA, 40 bp, synthetic construct
  source 1..40: mol_type = other DNA
ttagggataacagggtaatccaagcagctggtggaagacc

SEQ ID NO: 183 - DNA, 41 bp, synthetic construct
  source 1..41: mol_type = other DNA
ttagggataacagggtaatcccttgcttctcagatgaaacc

SEQ ID NO: 184 - DNA, 40 bp, synthetic construct
  source 1..40: mol_type = other DNA
ttagggataacagggtaatccctccccgcctcccgctctc

SEQ ID NO: 185 - DNA, 40 bp, synthetic construct
  source 1..40: mol_type = other DNA
ttagggataacagggtaatccattacccaggggagccttc

SEQ ID NO: 186 - DNA, 40 bp, synthetic construct
  source 1..40: mol_type = other DNA
ttagggataacagggtaatcaggtgaagccaaacctgctc

SEQ ID NO: 187 - DNA, 40 bp, synthetic construct
  source 1..40: mol_type = other DNA
ttagggataacagggtaatcggactgataccccagctcag

SEQ ID NO: 188 - DNA, 42 bp, synthetic construct
  source 1..42: mol_type = other DNA
ttagggataacagggtaatccccaagtaaatgagtctcaacg

SEQ ID NO: 189 - DNA, 41 bp, synthetic construct
  source 1..41: mol_type = other DNA
ttagggataacagggtaatcccagagttcatggatgcactg

SEQ ID NO: 190 - DNA, 40 bp, synthetic construct
  source 1..40: mol_type = other DNA
ttagggataacagggtaatcccaggtacaggggcgaggtc

SEQ ID NO: 191 - DNA, 41 bp, synthetic construct
  source 1..41: mol_type = other DNA
ttagggataacagggtaatcaagcacctccatctctttgtc

SEQ ID NO: 192 - DNA, 41 bp, synthetic construct
  source 1..41: mol_type = other DNA
ttagggataacagggtaatctccacagtgagctcgatcctc

SEQ ID NO: 193 - DNA, 45 bp, synthetic construct
  source 1..45: mol_type = other DNA
ttagggataacagggtaatctctgaactatttatggacaacagtc

SEQ ID NO: 194 - DNA, 40 bp, synthetic construct
  source 1..40: mol_type = other DNA
ttagggataacagggtaatctgctctgagaaaggcattag

SEQ ID NO: 195 - DNA, 40 bp, synthetic construct
  source 1..40: mol_type = other DNA
ttagggataacagggtaatcttacggtagtgggggaaggc

SEQ ID NO: 196 - DNA, 41 bp, synthetic construct
  source 1..41: mol_type = other DNA
ttagggataacagggtaatctttgacctcaggtttctaacg

SEQ ID NO: 197 - DNA, 41 bp, synthetic construct
  source 1..41: mol_type = other DNA
ttagggataacagggtaatctctcagggtacaaattctcag

SEQ ID NO: 198 - DNA, 42 bp, synthetic construct
  source 1..42: mol_type = other DNA
ttagggataacagggtaatccgacatcgcgatggcccagctc

SEQ ID NO: 199 - DNA, 41 bp, synthetic construct
  source 1..41: mol_type = other DNA
ttagggataacagggtaatcgtgcacgacgctgcccgggag

SEQ ID NO: 200 - DNA, 41 bp, synthetic construct
  source 1..41: mol_type = other DNA
ttagggataacagggtaatctctacacaagcttcctttccg

SEQ ID NO: 201 - DNA, 40 bp, synthetic construct
  source 1..40: mol_type = other DNA
ttagggataacagggtaatccccctgcaaacttcgtcctc

SEQ ID NO: 202 - DNA, 40 bp, synthetic construct
  source 1..40: mol_type = other DNA
ttagggataacagggtaatcggctgcggagagggggagag

SEQ ID NO: 203 - DNA, 43 bp, synthetic construct
  source 1..43: mol_type = other DNA
ttagggataacagggtaatcgataatccattgcctgtctaaag

SEQ ID NO: 204 - DNA, 43 bp, synthetic construct
  source 1..43: mol_type = other DNA
ttagggataacagggtaatctgttgactttttagttttacttt

SEQ ID NO: 205 - DNA, 40 bp, synthetic construct
  source 1..40: mol_type = other DNA
ttagggataacagggtaatcctgtgggctgaaggctgttc

SEQ ID NO: 206 - DNA, 40 bp, synthetic construct
  source 1..40: mol_type = other DNA
ttagggataacagggtaatcagcgcaacaagcccactgtc

SEQ ID NO: 207 - DNA, 40 bp, synthetic construct
  source 1..40: mol_type = other DNA
ttagggataacagggtaatcgttccatctcccacttgtcg

SEQ ID NO: 208 - DNA, 40 bp, synthetic construct
  source 1..40: mol_type = other DNA
ttagggataacagggtaatctaatgccagcagacgccttg

SEQ ID NO: 209 - DNA, 40 bp, synthetic construct
  source 1..40: mol_type = other DNA
ttagggataacagggtaatcacccactgaaaagcacttcc

SEQ ID NO: 210 - DNA, 40 bp, synthetic construct
  source 1..40: mol_type = other DNA
ttagggataacagggtaatccggcgcgagaccctctcttc

SEQ ID NO: 211 - DNA, 40 bp, synthetic construct
  source 1..40: mol_type = other DNA
ttagggataacagggtaatcgtgttgaagtcctcgttgtc

SEQ ID NO: 212 - DNA, 40 bp, synthetic construct
  source 1..40: mol_type = other DNA
ttagggataacagggtaatccgccctgagacctcctaggc

SEQ ID NO: 213 - DNA, 42 bp, synthetic construct
  source 1..42: mol_type = other DNA
ttagggataacagggtaatcacctgttaagtttgtatgcaac

SEQ ID NO: 214 - DNA, 42 bp, synthetic construct
  source 1..42: mol_type = other DNA
ttagggataacagggtaatcgtgaagatgacaatcatgttgc

SEQ ID NO: 215 - DNA, 45 bp, synthetic construct
  source 1..45: mol_type = other DNA
ttagggataacagggtaatcaatttgccccgatgtaataaatatg

SEQ ID NO: 216 - DNA, 43 bp, synthetic construct
  source 1..43: mol_type = other DNA
ttagggataacagggtaatctccaataaattctcagatccagg

SEQ ID NO: 217 - DNA, 42 bp, synthetic construct
  source 1..42: mol_type = other DNA
ttagggataacagggtaatctaaatggctacgacccagttac

SEQ ID NO: 218 - DNA, 46 bp, synthetic construct
  source 1..46: mol_type = other DNA
ttagggataacagggtaatcggttaagaaaactgttccaatacatg

SEQ ID NO: 219 - DNA, 41 bp, synthetic construct
  source 1..41: mol_type = other DNA
ttagggataacagggtaatcacagtttgacagttaaaggca

SEQ ID NO: 220 - DNA, 44 bp, synthetic construct
  source 1..44: mol_type = other DNA
ttagggataacagggtaatcatacattcttcataccaggaccag

SEQ ID NO: 221 - DNA, 40 bp, synthetic construct
  source 1..40: mol_type = other DNA
ttagggataacagggtaatctcggttggctttgtctttat

SEQ ID NO: 222 - DNA, 41 bp, synthetic construct
  source 1..41: mol_type = other DNA
ttagggataacagggtaatcgttcttcatcagctgtactcc

SEQ ID NO: 223 - DNA, 41 bp, synthetic construct
  source 1..41: mol_type = other DNA
ttagggataacagggtaatcgttggtagaagacttggatcg

SEQ ID NO: 224 - DNA, 46 bp, synthetic construct
  source 1..46: mol_type = other DNA
ttagggataacagggtaatctctaaaatgattcaatcaaactgcag

SEQ ID NO: 225 - DNA, 45 bp, synthetic construct
  source 1..45: mol_type = other DNA
ttagggataacagggtaatcgaaatgaactgatttgtgaatatgc

SEQ ID NO: 226 - DNA, 40 bp, synthetic construct
  source 1..40: mol_type = other DNA
ttagggataacagggtaatccggggtgttggagttcatgg

SEQ ID NO: 227 - DNA, 43 bp, synthetic construct
  source 1..43: mol_type = other DNA
ttagggataacagggtaatcatgcagtaaatggctatctccag

SEQ ID NO: 228 - DNA, 40 bp, synthetic construct
  source 1..40: mol_type = other DNA
ttagggataacagggtaatccaatctctttgtccgtggtg

SEQ ID NO: 229 - DNA, 40 bp, synthetic construct
  source 1..40: mol_type = other DNA
ttagggataacagggtaatctgtggccaaacccatgaagg

SEQ ID NO: 230 - DNA, 40 bp, synthetic construct
  source 1..40: mol_type = other DNA
ttagggataacagggtaatcggtcactgacagccctctgg

SEQ ID NO: 231 - DNA, 41 bp, synthetic construct
  source 1..41: mol_type = other DNA
ttagggataacagggtaatcggcatggactacgcgcaatgc

SEQ ID NO: 232 - DNA, 40 bp, synthetic construct
  source 1..40: mol_type = other DNA
ttagggataacagggtaatcctgtgcggcgtgggctcccg

SEQ ID NO: 233 - DNA, 40 bp, synthetic construct
  source 1..40: mol_type = other DNA
ttagggataacagggtaatccccccacggaaggtcctgag

SEQ ID NO: 234 - DNA, 40 bp, synthetic construct
  source 1..40: mol_type = other DNA
ttagggataacagggtaatctggctgtgtcctgggctcgc

SEQ ID NO: 235 - DNA, 40 bp, synthetic construct
  source 1..40: mol_type = other DNA
ttagggataacagggtaatcggcaggtggggcaggagacc

SEQ ID NO: 236 - DNA, 44 bp, synthetic construct
  source 1..44: mol_type = other DNA
ttagggataacagggtaatcattcaggagcttccaaatagtatg

SEQ ID NO: 237 - DNA, 45 bp, synthetic construct
  source 1..45: mol_type = other DNA
ttagggataacagggtaatccagtttaaaaatcacatggcatctg

SEQ ID NO: 238 - DNA, 40 bp, synthetic construct
  source 1..40: mol_type = other DNA
ttagggataacagggtaatccgctgtgagacatgtctgag

SEQ ID NO: 239 - DNA, 40 bp, synthetic construct
  source 1..40: mol_type = other DNA
ttagggataacagggtaatcactcggcactgggctctctg

SEQ ID NO: 240 - DNA, 40 bp, synthetic construct
  source 1..40: mol_type = other DNA
ttagggataacagggtaatctcgacgccacttcatgttcc

SEQ ID NO: 241 - DNA, 40 bp, synthetic construct
  source 1..40: mol_type = other DNA
ttagggataacagggtaatcccactgggctacgtcatccc

SEQ ID NO: 242 - DNA, 42 bp, synthetic construct
  source 1..42: mol_type = other DNA
ttagggataacagggtaatcccaaagaggactttcgctatcg

SEQ ID NO: 243 - DNA, 40 bp, synthetic construct
  source 1..40: mol_type = other DNA
ttagggataacagggtaatcccaccctggaaactctatac

SEQ ID NO: 244 - DNA, 42 bp, synthetic construct
  source 1..42: mol_type = other DNA
ttagggataacagggtaatcagggttatggtgcacattatcc

SEQ ID NO: 245 - DNA, 41 bp, synthetic construct
  source 1..41: mol_type = other DNA
ttagggataacagggtaatcgtctccccgcctgaagagcac

SEQ ID NO: 246 - DNA, 41 bp, synthetic construct
  source 1..41: mol_type = other DNA
ttagggataacagggtaatcgcaactgatagacgtaatccc

SEQ ID NO: 247 - DNA, 42 bp, synthetic construct
  source 1..42: mol_type = other DNA
ttagggataacagggtaatcatagtggatcccaacggactgg

SEQ ID NO: 248 - DNA, 42 bp, synthetic construct
  source 1..42: mol_type = other DNA
ttagggataacagggtaatcgctctggacattttctcatagg

SEQ ID NO: 249 - DNA, 42 bp, synthetic construct
  source 1..42: mol_type = other DNA
ttagggataacagggtaatcgattattttgccctgttgtctc

SEQ ID NO: 250 - DNA, 42 bp, synthetic construct
  source 1..42: mol_type = other DNA
ttagggataacagggtaatcgtgaggccagaaggaaccatcg

SEQ ID NO: 251 - DNA, 40 bp, synthetic construct
  source 1..40: mol_type = other DNA
ttagggataacagggtaatcgggttcagcagaaagaggac

SEQ ID NO: 252 - DNA, 42 bp, synthetic construct
  source 1..42: mol_type = other DNA
ttagggataacagggtaatcgactgctaccttatatcccttc

SEQ ID NO: 253 - DNA, 40 bp, synthetic construct
  source 1..40: mol_type = other DNA
ttagggataacagggtaatcgggaattcaaggtagtccag

SEQ ID NO: 254 - DNA, 40 bp, synthetic construct
  source 1..40: mol_type = other DNA
ttagggataacagggtaatcggggtccccatccattcttc

SEQ ID NO: 255 - DNA, 40 bp, synthetic construct
  source 1..40: mol_type = other DNA
ttagggataacagggtaatcccccacgcccaaccctccac

SEQ ID NO: 256 - DNA, 40 bp, synthetic construct
  source 1..40: mol_type = other DNA
ttagggataacagggtaatcagtaaagccacctcacgaac

SEQ ID NO: 257 - DNA, 40 bp, synthetic construct
  source 1..40: mol_type = other DNA
ttagggataacagggtaatcgccagtggctgaaattggtg

SEQ ID NO: 258 - DNA, 42 bp, synthetic construct
  source 1..42: mol_type = other DNA
ttagggataacagggtaatcgacttttggtgataggagtctg

SEQ ID NO: 259 - DNA, 42 bp, synthetic construct
  source 1..42: mol_type = other DNA
ttagggataacagggtaatcactctttatttgtccccttgcc

SEQ ID NO: 260 - DNA, 40 bp, synthetic construct
  source 1..40: mol_type = other DNA
ttagggataacagggtaatcctcagggcacaagggagcag

SEQ ID NO: 261 - DNA, 42 bp, synthetic construct
  source 1..42: mol_type = other DNA
ttagggataacagggtaatcttgtctctgagtccactaaaag

SEQ ID NO: 262 - DNA, 42 bp, synthetic construct
  source 1..42: mol_type = other DNA
ttagggataacagggtaatcaaaaggagagcgtatccaagag

SEQ ID NO: 263 - DNA, 40 bp, synthetic construct
  source 1..40: mol_type = other DNA
ttagggataacagggtaatctccgtggccatctggatgcg

SEQ ID NO: 264 - DNA, 42 bp, synthetic construct
  source 1..42: mol_type = other DNA
ttagggataacagggtaatcgggctgcatgttagaatcatcc

SEQ ID NO: 265 - DNA, 40 bp, synthetic construct
  source 1..40: mol_type = other DNA
ttagggataacagggtaatctccccatcccaggagcttac

SEQ ID NO: 266 - DNA, 42 bp, synthetic construct
  source 1..42: mol_type = other DNA
ttagggataacagggtaatcgagtttagagcttggctttatg

SEQ ID NO: 267 - DNA, 40 bp, synthetic construct
  source 1..40: mol_type = other DNA
ttagggataacagggtaatcgacacaacacaaaatagccg

SEQ ID NO: 268 - DNA, 42 bp, synthetic construct
  source 1..42: mol_type = other DNA
ttagggataacagggtaatcactccaggataatacacatcac

SEQ ID NO: 269 - DNA, 40 bp, synthetic construct
  source 1..40: mol_type = other DNA
ttagggataacagggtaatctgggcaaagagggctccagc

SEQ ID NO: 270 - DNA, 41 bp, synthetic construct
  source 1..41: mol_type = other DNA
ttagggataacagggtaatcggtctgacgggtagagtgtgc

SEQ ID NO: 271 - DNA, 40 bp, synthetic construct
  source 1..40: mol_type = other DNA
ttagggataacagggtaatcggtgcccaggtcagtggatc

SEQ ID NO: 272 - DNA, 43 bp, synthetic construct
  source 1..43: mol_type = other DNA
ttagggataacagggtaatcttcaaattctggttgaaagatgg

SEQ ID NO: 273 - DNA, 40 bp, synthetic construct
  source 1..40: mol_type = other DNA
ttagggataacagggtaatcgtgcaggccaacttgttcag

SEQ ID NO: 274 - DNA, 41 bp, synthetic construct
  source 1..41: mol_type = other DNA
ttagggataacagggtaatccatgtgatgtcatctctcctc

SEQ ID NO: 275 - DNA, 40 bp, synthetic construct
  source 1..40: mol_type = other DNA
ttagggataacagggtaatcgctgcctttgaccatgaagg

SEQ ID NO: 276 - DNA, 43 bp, synthetic construct
  source 1..43: mol_type = other DNA
ttagggataacagggtaatcgaaccatcttttaactcaggtac

SEQ ID NO: 277 - DNA, 41 bp, synthetic construct
  source 1..41: mol_type = other DNA
ttagggataacagggtaatcacggcattttgagtgttagac

SEQ ID NO: 278 - DNA, 43 bp, synthetic construct
  source 1..43: mol_type = other DNA
ttagggataacagggtaatcgcctcagattcacttttatcacc

SEQ ID NO: 279 - DNA, 40 bp, synthetic construct
  source 1..40: mol_type = other DNA
ttagggataacagggtaatcctccaccgcttcttgtcctg

SEQ ID NO: 280 - DNA, 43 bp, synthetic construct
  source 1..43: mol_type = other DNA
ttagggataacagggtaatctactgcctcttgcttctcttttc

SEQ ID NO: 281 - DNA, 41 bp, synthetic construct
  source 1..41: mol_type = other DNA
ttagggataacagggtaatcagaggtgggcccaggggtcag

SEQ ID NO: 282 - DNA, 42 bp, synthetic construct
  source 1..42: mol_type = other DNA
ttagggataacagggtaatcttgccacaggtctccccaaggc

SEQ ID NO: 283 - DNA, 41 bp, synthetic construct
  source 1..41: mol_type = other DNA
ttagggataacagggtaatcccttaacccctcctcccagag

SEQ ID NO: 284 - DNA, 40 bp, synthetic construct
  source 1..40: mol_type = other DNA
ttagggataacagggtaatcggtccccaggcctctgattc

SEQ ID NO: 285 - DNA, 40 bp, synthetic construct
  source 1..40: mol_type = other DNA
ttagggataacagggtaatcgccctgtcgtctctccagcc

SEQ ID NO: 286 - DNA, 40 bp, synthetic construct
  source 1..40: mol_type = other DNA
ttagggataacagggtaatcctgttcacttgtgccctgac

SEQ ID NO: 287 - DNA, 41 bp, synthetic construct
  source 1..41: mol_type = other DNA
ttagggataacagggtaatcatacggccaggcattgaagtc

SEQ ID NO: 288 - DNA, 40 bp, synthetic construct
  source 1..40: mol_type = other DNA
ttagggataacagggtaatctgccctggtaggttttctgg

SEQ ID NO: 289 - DNA, 41 bp, synthetic construct
  source 1..41: mol_type = other DNA
ttagggataacagggtaatcccagatgaagctcccagaatg

SEQ ID NO: 290 - DNA, 40 bp, synthetic construct
  source 1..40: mol_type = other DNA
ttagggataacagggtaatccctctgactgctcttttcac

SEQ ID NO: 291 - DNA, 40 bp, synthetic construct
  source 1..40: mol_type = other DNA
ttagggataacagggtaatctccaggtccccagcccaacc

SEQ ID NO: 292 - DNA, 40 bp, synthetic construct
  source 1..40: mol_type = other DNA
ttagggataacagggtaatcggaagcgaaaattccatggg

SEQ ID NO: 293 - DNA, 43 bp, synthetic construct
  source 1..43: mol_type = other DNA
ttagggataacagggtaatccgcttcccacaggtctctgctag

SEQ ID NO: 294 - DNA, 40 bp, synthetic construct
  source 1..40: mol_type = other DNA
ttagggataacagggtaatcaagtgtctcatgctggatcc

SEQ ID NO: 295 - DNA, 40 bp, synthetic construct
  source 1..40: mol_type = other DNA
ttagggataacagggtaatcttgtggagtgcaagtgaaag

SEQ ID NO: 296 - DNA, 42 bp, synthetic construct
  source 1..42: mol_type = other DNA
ttagggataacagggtaatctgtacatgggaaaacataacct

SEQ ID NO: 297 - DNA, 42 bp, synthetic construct
  source 1..42: mol_type = other DNA
ttagggataacagggtaatcggattgcagattgggccttggg

SEQ ID NO: 298 - DNA, 40 bp, synthetic construct
  source 1..40: mol_type = other DNA
ttagggataacagggtaatccggcccccaacgctgcccct

SEQ ID NO: 299 - DNA, 40 bp, synthetic construct
  source 1..40: mol_type = other DNA
ttagggataacagggtaatcggctttggtgagatccattg

SEQ ID NO: 300 - DNA, 40 bp, synthetic construct
  source 1..40: mol_type = other DNA
ttagggataacagggtaatctggttattccagagggactg

SEQ ID NO: 301 - DNA, 40 bp, synthetic construct
  source 1..40: mol_type = other DNA
ttagggataacagggtaatctggaataaccagctgtcctc

SEQ ID NO: 302 - DNA, 40 bp, synthetic construct
  source 1..40: mol_type = other DNA
ttagggataacagggtaatcatccctaacaacacagaagc

SEQ ID NO: 303 - DNA, 46 bp, synthetic construct
  source 1..46: mol_type = other DNA
ttagggataacagggtaatcctagagtttttccaagaaccaagttc

SEQ ID NO: 304 - DNA, 40 bp, synthetic construct
  source 1..40: mol_type = other DNA
ttagggataacagggtaatccggcctccctctggaaatcc

SEQ ID NO: 305 - DNA, 40 bp, synthetic construct
  source 1..40: mol_type = other DNA
ttagggataacagggtaatccaaatatggtcgacggtcca

SEQ ID NO: 306 - DNA, 40 bp, synthetic construct
  source 1..40: mol_type = other DNA
ttagggataacagggtaatccgtcagggacacaactgcca

SEQ ID NO: 307 - DNA, 40 bp, synthetic construct
  source 1..40: mol_type = other DNA
ttagggataacagggtaatcaacctcagcccgaccaagga

SEQ ID NO: 308 - DNA, 40 bp, synthetic construct
  source 1..40: mol_type = other DNA
ttagggataacagggtaatctttctttctgcctcatgctc

SEQ ID NO: 309 - DNA, 40 bp, synthetic construct
  source 1..40: mol_type = other DNA
ttagggataacagggtaatcgtggctcaagtgggctgaaa

SEQ ID NO: 310 - DNA, 43 bp, synthetic construct
  source 1..43: mol_type = other DNA
ttagggataacagggtaatctttggtcagcagcaggagagctt

SEQ ID NO: 311 - DNA, 40 bp, synthetic construct
  source 1..40: mol_type = other DNA
ttagggataacagggtaatcactttccaagttgggcatat

SEQ ID NO: 312 - DNA, 40 bp, synthetic construct
  source 1..40: mol_type = other DNA
ttagggataacagggtaatcggacatgagggcctccttct

SEQ ID NO: 313 - DNA, 41 bp, synthetic construct
  source 1..41: mol_type = other DNA
ttagggataacagggtaatcatcctccttgcccagaccaaa

SEQ ID NO: 314 - DNA, 46 bp, synthetic construct
  source 1..46: mol_type = other DNA
ttagggataacagggtaatccctcatagctctcgatgatcttccag

SEQ ID NO: 315 - DNA, 40 bp, synthetic construct
  source 1..40: mol_type = other DNA
ttagggataacagggtaatcgatggacatgcaggcgacca

SEQ ID NO: 316 - DNA, 40 bp, synthetic construct
  source 1..40: mol_type = other DNA
ttagggataacagggtaatcgcaccgtcaccttgtggaag

SEQ ID NO: 317 - DNA, 40 bp, synthetic construct
  source 1..40: mol_type = other DNA
ttagggataacagggtaatccagagccgttgatgaatgtc

SEQ ID NO: 318 - DNA, 40 bp, synthetic construct
  source 1..40: mol_type = other DNA
ttagggataacagggtaatcgcaaggatccagaggaggaa

SEQ ID NO: 319 - DNA, 40 bp, synthetic construct
  source 1..40: mol_type = other DNA
ttagggataacagggtaatctgccaacaccacctgcccaa

SEQ ID NO: 320 - DNA, 40 bp, synthetic construct
  source 1..40: mol_type = other DNA
ttagggataacagggtaatcgcaaccctgcttgcaggatg

SEQ ID NO: 321 - DNA, 41 bp, synthetic construct
  source 1..41: mol_type = other DNA
ttagggataacagggtaatccaccacagagtccattatgat

SEQ ID NO: 322 - DNA, 41 bp, synthetic construct
  source 1..41: mol_type = other DNA
ttagggataacagggtaatcccatcttttctggggatgtcc

SEQ ID NO: 323 - DNA, 45 bp, synthetic construct
  source 1..45: mol_type = other DNA
ttagggataacagggtaatcgttatctgtttctttctcctctgaa

SEQ ID NO: 324 - DNA, 40 bp, synthetic construct
  source 1..40: mol_type = other DNA
ttagggataacagggtaatcgaaggactccacttccacag

SEQ ID NO: 325 - DNA, 40 bp, synthetic construct
  source 1..40: mol_type = other DNA
ttagggataacagggtaatctcagcctcaacgagtgcttc

SEQ ID NO: 326 - DNA, 40 bp, synthetic construct
  source 1..40: mol_type = other DNA
ttagggataacagggtaatcgccacgagttgttgaggaag

SEQ ID NO: 327 - DNA, 40 bp, synthetic construct
  source 1..40: mol_type = other DNA
ttagggataacagggtaatcccctgcccactgtgttactg

SEQ ID NO: 328 - DNA, 42 bp, synthetic construct
  source 1..42: mol_type = other DNA
ttagggataacagggtaatctttgttctggcggtgttttgaa

SEQ ID NO: 329 - DNA, 40 bp, synthetic construct
  source 1..40: mol_type = other DNA
ttagggataacagggtaatcatggcatcaaagcagaggac

SEQ ID NO: 330 - DNA, 41 bp, synthetic construct
  source 1..41: mol_type = other DNA
ttagggataacagggtaatcaagtctggcttcttggtcgtg

SEQ ID NO: 331 - DNA, 43 bp, synthetic construct
  source 1..43: mol_type = other DNA
ttagggataacagggtaatcactgcataggggtcttcttaatc

SEQ ID NO: 332 - DNA, 40 bp, synthetic construct
  source 1..40: mol_type = other DNA
ttagggataacagggtaatcggcctgcttatctgttcctc

SEQ ID NO: 333 - DNA, 41 bp, synthetic construct
  source 1..41: mol_type = other DNA
ttagggataacagggtaatcgatgtgctgggtccttttcag

SEQ ID NO: 334 - DNA, 40 bp, synthetic construct
  source 1..40: mol_type = other DNA
ttagggataacagggtaatcaaacgagaagcacatcaacc

SEQ ID NO: 335 - DNA, 41 bp, synthetic construct
  source 1..41: mol_type = other DNA
ttagggataacagggtaatcaaaccaaacaacttccacctc

SEQ ID NO: 336 - DNA, 41 bp, synthetic construct
  source 1..41: mol_type = other DNA
ttagggataacagggtaatccctttcggaagagatgtctaa

SEQ ID NO: 337 - DNA, 42 bp, synthetic construct
  source 1..42: mol_type = other DNA
ttagggataacagggtaatcaggacaggtataaaggaaacac

SEQ ID NO: 338 - DNA, 42 bp, synthetic construct
  source 1..42: mol_type = other DNA
ttagggataacagggtaatccttctgtgagctccaaaccaag

SEQ ID NO: 339 - DNA, 40 bp, synthetic construct
  source 1..40: mol_type = other DNA
ttagggataacagggtaatcggaggggtgcagcattggtg

SEQ ID NO: 340 - DNA, 44 bp, synthetic construct
  source 1..44: mol_type = other DNA
ttagggataacagggtaatcaagctttgttttcttcacattcta

SEQ ID NO: 341 - DNA, 40 bp, synthetic construct
  source 1..40: mol_type = other DNA
ttagggataacagggtaatccaagaccaacctccatgtgg

SEQ ID NO: 342 - DNA, 41 bp, synthetic construct
  source 1..41: mol_type = other DNA
ttagggataacagggtaatcttgattggggaactgtaacgc

SEQ ID NO: 343 - DNA, 41 bp, synthetic construct
  source 1..41: mol_type = other DNA
ttagggataacagggtaatcttggagggcagcctcttcacc

SEQ ID NO: 344 - DNA, 41 bp, synthetic construct
  source 1..41: mol_type = other DNA
ttagggataacagggtaatctttggctctgcaccctaacgg

SEQ ID NO: 345 - DNA, 38 bp, synthetic construct
  source 1..38: mol_type = other DNA
ttagggataacagggtaatcagcctctccacgctccct

SEQ ID NO: 346 - DNA, 40 bp, synthetic construct
  source 1..40: mol_type = other DNA
ttagggataacagggtaatcacaggcctgagagtggagct

SEQ ID NO: 347 - DNA, 40 bp, synthetic construct
  source 1..40: mol_type = other DNA
ttagggataacagggtaatcctcaactccaggccaggtgt

SEQ ID NO: 348 - DNA, 38 bp, synthetic construct
  source 1..38: mol_type = other DNA
ttagggataacagggtaatcaggtcctcgtgcctgcca

SEQ ID NO: 349 - DNA, 39 bp, synthetic construct
  source 1..39: mol_type = other DNA
ttagggataacagggtaatcggcacgaggacctgtggga

SEQ ID NO: 350 - DNA, 43 bp, synthetic construct
  source 1..43: mol_type = other DNA
ttagggataacagggtaatcgccctttttgtggggtaggacag

SEQ ID NO: 351 - DNA, 42 bp, synthetic construct
  source 1..42: mol_type = other DNA
ttagggataacagggtaatcctctagctccttgtcggtggtg

SEQ ID NO: 352 - DNA, 38 bp, synthetic construct
  source 1..38: mol_type = other DNA
ttagggataacagggtaatctgcccgcctcgtcagcct

SEQ ID NO: 353 - DNA, 42 bp, synthetic construct
  source 1..42: mol_type = other DNA
ttagggataacagggtaatcgttggagctcatggacgcgttg

SEQ ID NO: 354 - DNA, 41 bp, synthetic construct
  source 1..41: mol_type = other DNA
ttagggataacagggtaatctcacagctctcagggacccaa

SEQ ID NO: 355 - DNA, 40 bp, synthetic construct
  source 1..40: mol_type = other DNA
ttagggataacagggtaatccaggacctccacctctgagc

SEQ ID NO: 356 - DNA, 39 bp, synthetic construct
  source 1..39: mol_type = other DNA
ttagggataacagggtaatcagggagcccgacgcgtaca

SEQ ID NO: 357 - DNA, 41 bp, synthetic construct
  source 1..41: mol_type = other DNA
ttagggataacagggtaatccttgacttgccggaagagcct

SEQ ID NO: 358 - DNA, 38 bp, synthetic construct
  source 1..38: mol_type = other DNA
ttagggataacagggtaatcctggtggaggcgctggac

SEQ ID NO: 359 - DNA, 44 bp, synthetic construct
  source 1..44: mol_type = other DNA
ttagggataacagggtaatctgactttccattctctgctggatg

SEQ ID NO: 360 - DNA, 43 bp, synthetic construct
  source 1..43: mol_type = other DNA
ttagggataacagggtaatcagatgagtatgcctgccgtgtga

SEQ ID NO: 361 - DNA, 41 bp, synthetic construct
  source 1..41: mol_type = other DNA
ttagggataacagggtaatcgttcagcaaatgccagtaggt

SEQ ID NO: 362 - DNA, 45 bp, synthetic construct
  source 1..45: mol_type = other DNA
ttagggataacagggtaatcaccatactctaccacatataggtcc

SEQ ID NO: 363 - DNA, 40 bp, synthetic construct
  source 1..40: mol_type = other DNA
ttagggataacagggtaatcaggtgactggatccacaacc

SEQ ID NO: 364 - DNA, 45 bp, synthetic construct
  source 1..45: mol_type = other DNA
ttagggataacagggtaatcgagattagatcctgaggaaaaccat

SEQ ID NO: 365 - DNA, 45 bp, synthetic construct
  source 1..45: mol_type = other DNA
ttagggataacagggtaatctgggagccatcttattatgccttgg

SEQ ID NO: 366 - DNA, 44 bp, synthetic construct
  source 1..44: mol_type = other DNA
ttagggataacagggtaatcctttgagtttgtatcttggatgcc

SEQ ID NO: 367 - DNA, 45 bp, synthetic construct
  source 1..45: mol_type = other DNA
ttagggataacagggtaatccgcatcaatacaaacttgcgcacat

SEQ ID NO: 368 - DNA, 42 bp, synthetic construct
  source 1..42: mol_type = other DNA
ttagggataacagggtaatcccatgaagggtgtcaccaaggc

SEQ ID NO: 369 - DNA, 44 bp, synthetic construct
  source 1..44: mol_type = other DNA
ttagggataacagggtaatccaagactaagtaggaccatgtagg

SEQ ID NO: 370 - DNA, 43 bp, synthetic construct
  source 1..43: mol_type = other DNA
ttagggataacagggtaatccctggttattgagtgagccccaa

SEQ ID NO: 371 - DNA, 44 bp, synthetic construct
  source 1..44: mol_type = other DNA
ttagggataacagggtaatctggtttcacatcgtcccccttttt

SEQ ID NO: 372 - DNA, 41 bp, synthetic construct
  source 1..41: mol_type = other DNA
ttagggataacagggtaatctgacccctcggaaaacaccct

SEQ ID NO: 373 - DNA, 40 bp, synthetic construct
  source 1..40: mol_type = other DNA
ttagggataacagggtaatcgcgttgtccccttcggtcac

SEQ ID NO: 374 - DNA, 38 bp, synthetic construct
  source 1..38: mol_type = other DNA
ttagggataacagggtaatcctggggtgggctgtgggc

SEQ ID NO: 375 - DNA, 40 bp, synthetic construct
  source 1..40: mol_type = other DNA
ttagggataacagggtaatcagcagcaggagaaggatgcc

SEQ ID NO: 376 - DNA, 41 bp, synthetic construct
  source 1..41: mol_type = other DNA
ttagggataacagggtaatcccccgagggccctcttgaagt

SEQ ID NO: 377 - DNA, 46 bp, synthetic construct
  source 1..46: mol_type = other DNA
ttagggataacagggtaatcactggatttagagtctctcagctggt

SEQ ID NO: 378 - DNA, 43 bp, synthetic construct
  source 1..43: mol_type = other DNA
ttagggataacagggtaatctgatggctcaaacacagcgacct

SEQ ID NO: 379 - DNA, 40 bp, synthetic construct
  source 1..40: mol_type = other DNA
ttagggataacagggtaatcgcacgatgtcccggcgcttg

SEQ ID NO: 380 - DNA, 56 bp, synthetic construct
  source 1..56: mol_type = other DNA
gtgactggagttcagacgtgtgctcttccgatctgatctgttagaaacctctccag

SEQ ID NO: 381 - DNA, 58 bp, synthetic construct
  source 1..58: mol_type = other DNA
gtgactggagttcagacgtgtgctcttccgatctatgtaactttgtatcctgttcctc

SEQ ID NO: 382 - DNA, 60 bp, synthetic construct
  source 1..60: mol_type = other DNA
gtgactggagttcagacgtgtgctcttccgatctgccgttgtacactcatcttcctaggg

SEQ ID NO: 383 - DNA, 54 bp, synthetic construct
  source 1..54: mol_type = other DNA
gtgactggagttcagacgtgtgctcttccgatctatggatccagacaactgttc

SEQ ID NO: 384 - DNA, 57 bp, synthetic construct
  source 1..57: mol_type = other DNA
gtgactggagttcagacgtgtgctcttccgatctgcttgctctgataggaaaatgag

SEQ ID NO: 385 - DNA, 54 bp, synthetic construct
  source 1..54: mol_type = other DNA
gtgactggagttcagacgtgtgctcttccgatctggagaagctcccaaccaagc

SEQ ID NO: 386 - DNA, 55 bp, synthetic construct
  source 1..55: mol_type = other DNA
gtgactggagttcagacgtgtgctcttccgatctcagaccatgagaggccctgcg

SEQ ID NO: 387 - DNA, 60 bp, synthetic construct
  source 1..60: mol_type = other DNA
gtgactggagttcagacgtgtgctcttccgatctgttaacgtcttccttctctctctgtc

SEQ ID NO: 388 - DNA, 55 bp, synthetic construct
  source 1..55: mol_type = other DNA
gtgactggagttcagacgtgtgctcttccgatctcccacacagcaaagcagaaac

SEQ ID NO: 389 - DNA, 56 bp, synthetic construct
  source 1..56: mol_type = other DNA
gtgactggagttcagacgtgtgctcttccgatcttccttctggccaccatgcgaag

SEQ ID NO: 390 - DNA, 56 bp, synthetic construct
  source 1..56: mol_type = other DNA
gtgactggagttcagacgtgtgctcttccgatctagccaatattgtctttgtgttc

SEQ ID NO: 391 - DNA, 54 bp, synthetic construct
  source 1..54: mol_type = other DNA
gtgactggagttcagacgtgtgctcttccgatcttgaaaacaccgcagcatgtc

SEQ ID NO: 392 - DNA, 57 bp, synthetic construct
  source 1..57: mol_type = other DNA
gtgactggagttcagacgtgtgctcttccgatctgaaaatgctggctgacctaaagc

SEQ ID NO: 393 - DNA, 54 bp, synthetic construct
  source 1..54: mol_type = other DNA
gtgactggagttcagacgtgtgctcttccgatctgtggctacatgttcctgatc

SEQ ID NO: 394 - DNA, 55 bp, synthetic construct
  source 1..55: mol_type = other DNA
gtgactggagttcagacgtgtgctcttccgatctgaaggataggacagggtgggc

SEQ ID NO: 395 - DNA, 60 bp, synthetic construct
  source 1..60: mol_type = other DNA
gtgactggagttcagacgtgtgctcttccgatctcataccctctcagcgtacccttgtcc

SEQ ID NO: 396 - DNA, 56 bp, synthetic construct
  source 1..56: mol_type = other DNA
gtgactggagttcagacgtgtgctcttccgatctgggtttgctccggagagacctg

SEQ ID NO: 397 - DNA, 59 bp, synthetic construct
  source 1..59: mol_type = other DNA
gtgactggagttcagacgtgtgctcttccgatctttggatcatattcgtccacaaaatg

SEQ ID NO: 398 - DNA, 54 bp, synthetic construct
  source 1..54: mol_type = other DNA
gtgactggagttcagacgtgtgctcttccgatctggcctgctgaaaatgactga

SEQ ID NO: 399 - DNA, 54 bp, synthetic construct
  source 1..54: mol_type = other DNA
gtgactggagttcagacgtgtgctcttccgatcttccccagtcctcatgtactg

SEQ ID NO: 400 - DNA, 59 bp, synthetic construct
  source 1..59: mol_type = other DNA
gtgactggagttcagacgtgtgctcttccgatctaggaagcaagtagtaattgatggag

SEQ ID NO: 401 - DNA, 54 bp, synthetic construct
  source 1..54: mol_type = other DNA
gtgactggagttcagacgtgtgctcttccgatcttgggcactgggtcaaagtct

SEQ ID NO: 402 - DNA, 58 bp, synthetic construct
  source 1..58: mol_type = other DNA
gtgactggagttcagacgtgtgctcttccgatctcaacccactgaggtatatgtatag

SEQ ID NO: 403 - DNA, 54 bp, synthetic construct
  source 1..54: mol_type = other DNA
gtgactggagttcagacgtgtgctcttccgatctacgcagtgctaaccaagttc

SEQ ID NO: 404 - DNA, 60 bp, synthetic construct
  source 1..60: mol_type = other DNA
gtgactggagttcagacgtgtgctcttccgatctgcaaaccacaaaagtatactccatgg

SEQ ID NO: 405 - DNA, 60 bp, synthetic construct
  source 1..60: mol_type = other DNA
gtgactggagttcagacgtgtgctcttccgatctgtaggctggatgaaaaattcacagtc

SEQ ID NO: 406 - DNA, 58 bp, synthetic construct
  source 1..58: mol_type = other DNA
gtgactggagttcagacgtgtgctcttccgatctctgacttggtggtaaacttttgag

SEQ ID NO: 407 - DNA, 59 bp, synthetic construct
  source 1..59: mol_type = other DNA
gtgactggagttcagacgtgtgctcttccgatctaaaatgacaaagaacagctcaaagc

SEQ ID NO: 408 - DNA, 57 bp, synthetic construct
  source 1..57: mol_type = other DNA
gtgactggagttcagacgtgtgctcttccgatctagcacttacctgtgactccatag

SEQ ID NO: 409 - DNA, 57 bp, synthetic construct
  source 1..57: mol_type = other DNA
gtgactggagttcagacgtgtgctcttccgatctagccttagataaaactgagcaag

SEQ ID NO: 410 - DNA, 57 bp, synthetic construct
  source 1..57: mol_type = other DNA
gtgactggagttcagacgtgtgctcttccgatctccaatattatggatcccaactgc

SEQ ID NO: 411 - DNA, 56 bp, synthetic construct
  source 1..56: mol_type = other DNA
gtgactggagttcagacgtgtgctcttccgatctctgcttctgtcatatcatccag

SEQ ID NO: 412 - DNA, 56 bp, synthetic construct
  source 1..56: mol_type = other DNA
gtgactggagttcagacgtgtgctcttccgatctggatcagcgagagtggcaggtg

SEQ ID NO: 413 - DNA, 55 bp, synthetic construct
  source 1..55: mol_type = other DNA
gtgactggagttcagacgtgtgctcttccgatctccttcatacacctccccaaag

SEQ ID NO: 414 - DNA, 55 bp, synthetic construct
  source 1..55: mol_type = other DNA
gtgactggagttcagacgtgtgctcttccgatctgggctgcggcatgatccttgg

SEQ ID NO: 415 - DNA, 55 bp, synthetic construct
  source 1..55: mol_type = other DNA
gtgactggagttcagacgtgtgctcttccgatcttgcagaacacacagcccattc

SEQ ID NO: 416 - DNA, 54 bp, synthetic construct
  source 1..54: mol_type = other DNA
gtgactggagttcagacgtgtgctcttccgatctaggacggttgaagaaattgc

SEQ ID NO: 417 - DNA, 54 bp, synthetic construct
  source 1..54: mol_type = other DNA
gtgactggagttcagacgtgtgctcttccgatctgactagtttggaacctgcag

SEQ ID NO: 418 - DNA, 57 bp, synthetic construct
  source 1..57: mol_type = other DNA
gtgactggagttcagacgtgtgctcttccgatctatactctccagaatcagccagtg

SEQ ID NO: 419 - DNA, 60 bp, synthetic construct
  source 1..60: mol_type = other DNA
gtgactggagttcagacgtgtgctcttccgatctagacacatatgctccttcagttgagg

SEQ ID NO: 420 - DNA, 59 bp, synthetic construct
  source 1..59: mol_type = other DNA
gtgactggagttcagacgtgtgctcttccgatctccttctccgcacattttacaagatg

SEQ ID NO: 421 - DNA, 54 bp, synthetic construct
  source 1..54: mol_type = other DNA
gtgactggagttcagacgtgtgctcttccgatcttccaccgccgtgtgcagctg

SEQ ID NO: 422 - DNA, 55 bp, synthetic construct
  source 1..55: mol_type = other DNA
gtgactggagttcagacgtgtgctcttccgatctccaccgggtctccagatgtgc

SEQ ID NO: 423 - DNA, 58 bp, synthetic construct
  source 1..58: mol_type = other DNA
gtgactggagttcagacgtgtgctcttccgatctaggagcagcgtagaaaggaagagg

SEQ ID NO: 424 - DNA, 60 bp, synthetic construct
  source 1..60: mol_type = other DNA
gtgactggagttcagacgtgtgctcttccgatctccaatgtcatgaaatgcagggacatg

SEQ ID NO: 425 - DNA, 56 bp, synthetic construct
  source 1..56: mol_type = other DNA
gtgactggagttcagacgtgtgctcttccgatctgaggttggagattcgagaaatg

SEQ ID NO: 426 - DNA, 57 bp, synthetic construct
  source 1..57: mol_type = other DNA
gtgactggagttcagacgtgtgctcttccgatctgtccccgatgtcattcgctgcag

SEQ ID NO: 427 - DNA, 57 bp, synthetic construct
  source 1..57: mol_type = other DNA
gtgactggagttcagacgtgtgctcttccgatctaccacagacgcaatcaccaccac

SEQ ID NO: 428 - DNA, 55 bp, synthetic construct
  source 1..55: mol_type = other DNA
gtgactggagttcagacgtgtgctcttccgatctgattttactttcccaaatcca

SEQ ID NO: 429 - DNA, 58 bp, synthetic construct
  source 1..58: mol_type = other DNA
gtgactggagttcagacgtgtgctcttccgatctgagtcatcatcattgctgataacg

SEQ ID NO: 430 - DNA, 55 bp, synthetic construct
  source 1..55: mol_type = other DNA
gtgactggagttcagacgtgtgctcttccgatctcaaaagcagcaagtccaactg

SEQ ID NO: 431 - DNA, 60 bp, synthetic construct
  source 1..60: mol_type = other DNA
gtgactggagttcagacgtgtgctcttccgatcttgagtcctcctcaccactgatgacag

SEQ ID NO: 432 - DNA, 56 bp, synthetic construct
  source 1..56: mol_type = other DNA
gtgactggagttcagacgtgtgctcttccgatctcacgatgtctctcctcttaatg

SEQ ID NO: 433 - DNA, 54 bp, synthetic construct
  source 1..54: mol_type = other DNA
gtgactggagttcagacgtgtgctcttccgatctccaggctcgtgtcccccaac

SEQ ID NO: 434 - DNA, 56 bp, synthetic construct
  source 1..56: mol_type = other DNA
gtgactggagttcagacgtgtgctcttccgatctgacagcaggtctcgcagctcac

SEQ ID NO: 435 - DNA, 60 bp, synthetic construct
  source 1..60: mol_type = other DNA
gtgactggagttcagacgtgtgctcttccgatctggaccttggctgcatgaagttttaac

SEQ ID NO: 436 - DNA, 54 bp, synthetic construct
  source 1..54: mol_type = other DNA
gtgactggagttcagacgtgtgctcttccgatctggaatgcctggtttatttgg

SEQ ID NO: 437 - DNA, 60 bp, synthetic construct
  source 1..60: mol_type = other DNA
gtgactggagttcagacgtgtgctcttccgatctatgtcatcttccaccttaaattctgg

SEQ ID NO: 438 - DNA, 57 bp, synthetic construct
  source 1..57: mol_type = other DNA
gtgactggagttcagacgtgtgctcttccgatctaagattttcaatctcctcttggg

SEQ ID NO: 439 - DNA, 60 bp, synthetic construct
  source 1..60: mol_type = other DNA
gtgactggagttcagacgtgtgctcttccgatcttaatatcccatgattgtctatgtttg

SEQ ID NO: 440 - DNA, 59 bp, synthetic construct
  source 1..59: mol_type = other DNA
gtgactggagttcagacgtgtgctcttccgatcttgacattcgtctacctcacagtgac

SEQ ID NO: 441 - DNA, 54 bp, synthetic construct
  source 1..54: mol_type = other DNA
gtgactggagttcagacgtgtgctcttccgatctgtggcctgtcgtccggtctg

SEQ ID NO: 442 - DNA, 56 bp, synthetic construct
  source 1..56: mol_type = other DNA
gtgactggagttcagacgtgtgctcttccgatctctcatagctgtcggccccacag

SEQ ID NO: 443 - DNA, 60 bp, synthetic construct
  source 1..60: mol_type = other DNA
gtgactggagttcagacgtgtgctcttccgatcttctttaaattcaccaatacctattcc

SEQ ID NO: 444 - DNA, 54 bp, synthetic construct
  source 1..54: mol_type = other DNA
gtgactggagttcagacgtgtgctcttccgatctggtgctgcccgcctctgacc

SEQ ID NO: 445 - DNA, 56 bp, synthetic construct
  source 1..56: mol_type = other DNA
gtgactggagttcagacgtgtgctcttccgatctagtgtgtactcttgcatcgtag

SEQ ID NO: 446 - DNA, 57 bp, synthetic construct
  source 1..57: mol_type = other DNA
gtgactggagttcagacgtgtgctcttccgatctctagttaggatgggggacatgtc

SEQ ID NO: 447 - DNA, 58 bp, synthetic construct
  source 1..58: mol_type = other DNA
gtgactggagttcagacgtgtgctcttccgatctaattgccccgcagccacccacctc

SEQ ID NO: 448 - DNA, 54 bp, synthetic construct
  source 1..54: mol_type = other DNA
gtgactggagttcagacgtgtgctcttccgatctgtgagcagctcagcctcacg

SEQ ID NO: 449 - DNA, 55 bp, synthetic construct
  source 1..55: mol_type = other DNA
gtgactggagttcagacgtgtgctcttccgatctccagcagcttggcatcaggtc

SEQ ID NO: 450 - DNA, 57 bp, synthetic construct
  source 1..57: mol_type = other DNA
gtgactggagttcagacgtgtgctcttccgatctgaacaatgttatgtcgcttgatg

SEQ ID NO: 451 - DNA, 55 bp, synthetic construct
  source 1..55: mol_type = other DNA
gtgactggagttcagacgtgtgctcttccgatcttccactcacacctgcctgttg

SEQ ID NO: 452 - DNA, 54 bp, synthetic construct
  source 1..54: mol_type = other DNA
gtgactggagttcagacgtgtgctcttccgatctggcaaatgttgatgtcttgg

SEQ ID NO: 453 - DNA, 60 bp, synthetic construct
  source 1..60: mol_type = other DNA
gtgactggagttcagacgtgtgctcttccgatctgggcaaacttgtggtagcagtggatg

SEQ ID NO: 454 - DNA, 56 bp, synthetic construct
  source 1..56: mol_type = other DNA
gtgactggagttcagacgtgtgctcttccgatctctcctggtctgtggaacccttc

SEQ ID NO: 455 - DNA, 55 bp, synthetic construct
  source 1..55: mol_type = other DNA
gtgactggagttcagacgtgtgctcttccgatctctgcagttctcagctcacagc

SEQ ID NO: 456 - DNA, 55 bp, synthetic construct
  source 1..55: mol_type = other DNA
gtgactggagttcagacgtgtgctcttccgatctatccttgctcctgtccttggc

SEQ ID NO: 457 - DNA, 54 bp, synthetic construct
  source 1..54: mol_type = other DNA
gtgactggagttcagacgtgtgctcttccgatctagccacaccccattcttgag

SEQ ID NO: 458 - DNA, 59 bp, synthetic construct
  source 1..59: mol_type = other DNA
gtgactggagttcagacgtgtgctcttccgatctagaaatcacgcatttatgttttctc

SEQ ID NO: 459 - DNA, 60 bp, synthetic construct
  source 1..60: mol_type = other DNA
gtgactggagttcagacgtgtgctcttccgatcttctgcatttatgaacccccagccttg

SEQ ID NO: 460 - DNA, 56 bp, synthetic construct
  source 1..56: mol_type = other DNA
gtgactggagttcagacgtgtgctcttccgatctgcccagcgtgtcctctctcctc

SEQ ID NO: 461 - DNA, 54 bp, synthetic construct
  source 1..54: mol_type = other DNA
gtgactggagttcagacgtgtgctcttccgatctgagggcccacagaggaggac

SEQ ID NO: 462 - DNA, 60 bp, synthetic construct
  source 1..60: mol_type = other DNA
gtgactggagttcagacgtgtgctcttccgatctatgcatgaacatttttctccaccttg

SEQ ID NO: 463 - DNA, 55 bp, synthetic construct
  source 1..55: mol_type = other DNA
gtgactggagttcagacgtgtgctcttccgatctttctgttctccttcactttcc

SEQ ID NO: 464 - DNA, 54 bp, synthetic construct
  source 1..54: mol_type = other DNA
gtgactggagttcagacgtgtgctcttccgatctccagcccacgctcttctcac

SEQ ID NO: 465 - DNA, 59 bp, synthetic construct
  source 1..59: mol_type = other DNA
gtgactggagttcagacgtgtgctcttccgatctccccaaaccccaatgaagagagacc

SEQ ID NO: 466 - DNA, 58 bp, synthetic construct
  source 1..58: mol_type = other DNA
gtgactggagttcagacgtgtgctcttccgatctcagaaaacagatctgtatttattt

SEQ ID NO: 467 - DNA, 56 bp, synthetic construct
  source 1..56: mol_type = other DNA
gtgactggagttcagacgtgtgctcttccgatctttgtggacaggttttgaaagat

SEQ ID NO: 468 - DNA, 57 bp, synthetic construct
  source 1..57: mol_type = other DNA
gtgactggagttcagacgtgtgctcttccgatctcatgctgtttaattgtgtggaag

SEQ ID NO: 469 - DNA, 54 bp, synthetic construct
  source 1..54: mol_type = other DNA
gtgactggagttcagacgtgtgctcttccgatctccgcggaaaggaaggggagg

SEQ ID NO: 470 - DNA, 54 bp, synthetic construct
  source 1..54: mol_type = other DNA
gtgactggagttcagacgtgtgctcttccgatctgtggcggagggactggggac

SEQ ID NO: 471 - DNA, 54 bp, synthetic construct
  source 1..54: mol_type = other DNA
gtgactggagttcagacgtgtgctcttccgatctcccactcctgaccctgtctc

SEQ ID NO: 472 - DNA, 55 bp, synthetic construct
  source 1..55: mol_type = other DNA
gtgactggagttcagacgtgtgctcttccgatctcccctttcatgccccttgtgg

SEQ ID NO: 473 - DNA, 54 bp, synthetic construct
  source 1..54: mol_type = other DNA
gtgactggagttcagacgtgtgctcttccgatctatggcttgcagctcctggtg

SEQ ID NO: 474 - DNA, 57 bp, synthetic construct
  source 1..57: mol_type = other DNA
gtgactggagttcagacgtgtgctcttccgatcttatgtaccatcgatgtctacatg

SEQ ID NO: 475 - DNA, 54 bp, synthetic construct
  source 1..54: mol_type = other DNA
gtgactggagttcagacgtgtgctcttccgatctcccgagacccccagcgctac

SEQ ID NO: 476 - DNA, 54 bp, synthetic construct
  source 1..54: mol_type = other DNA
gtgactggagttcagacgtgtgctcttccgatctccacagcagggcttcttcag

SEQ ID NO: 477 - DNA, 56 bp, synthetic construct
  source 1..56: mol_type = other DNA
gtgactggagttcagacgtgtgctcttccgatctttgaaggagacggactggtgag

SEQ ID NO: 478 - DNA, 56 bp, synthetic construct
  source 1..56: mol_type = other DNA
gtgactggagttcagacgtgtgctcttccgatcttcaccacgaaatccttggtctc

SEQ ID NO: 479 - DNA, 55 bp, synthetic construct
  source 1..55: mol_type = other DNA
gtgactggagttcagacgtgtgctcttccgatctgttagtgagccaggtaatgag

SEQ ID NO: 480 - DNA, 57 bp, synthetic construct
  source 1..57: mol_type = other DNA
gtgactggagttcagacgtgtgctcttccgatcttcatcactcgagtcccgtctacc

SEQ ID NO: 481 - DNA, 54 bp, synthetic construct
  source 1..54: mol_type = other DNA
gtgactggagttcagacgtgtgctcttccgatcttaggtgctgtcacattcaac

SEQ ID NO: 482 - DNA, 58 bp, synthetic construct
  source 1..58: mol_type = other DNA
gtgactggagttcagacgtgtgctcttccgatctggtcagtgggattgtaacaaccag

SEQ ID NO: 483 - DNA, 55 bp, synthetic construct
  source 1..55: mol_type = other DNA
gtgactggagttcagacgtgtgctcttccgatctcactgtcaccccttccttggc

SEQ ID NO: 484 - DNA, 56 bp, synthetic construct
  source 1..56: mol_type = other DNA
gtgactggagttcagacgtgtgctcttccgatcttcatgtattggtctctcatggc

SEQ ID NO: 485 - DNA, 60 bp, synthetic construct
  source 1..60: mol_type = other DNA
gtgactggagttcagacgtgtgctcttccgatctaaaacaagtggttatagatggtgaaa

SEQ ID NO: 486 - DNA, 57 bp, synthetic construct
  source 1..57: mol_type = other DNA
gtgactggagttcagacgtgtgctcttccgatctggttctggattagctggattgtc

SEQ ID NO: 487 - DNA, 58 bp, synthetic construct
  source 1..58: mol_type = other DNA
gtgactggagttcagacgtgtgctcttccgatcttgtgaaatgactgagtacaaactg

SEQ ID NO: 488 - DNA, 55 bp, synthetic construct
  source 1..55: mol_type = other DNA
gtgactggagttcagacgtgtgctcttccgatctaggaagagcacagtcactttg

SEQ ID NO: 489 - DNA, 54 bp, synthetic construct
  source 1..54: mol_type = other DNA
gtgactggagttcagacgtgtgctcttccgatctagtggcccgcttctgtctcc

SEQ ID NO: 490 - DNA, 60 bp, synthetic construct
  source 1..60: mol_type = other DNA
gtgactggagttcagacgtgtgctcttccgatctcagcattactctgtgtcccgttaaac

SEQ ID NO: 491 - DNA, 59 bp, synthetic construct
  source 1..59: mol_type = other DNA
gtgactggagttcagacgtgtgctcttccgatctcaggccttcccctctctttctatag

SEQ ID NO: 492 - DNA, 55 bp, synthetic construct
  source 1..55: mol_type = other DNA
gtgactggagttcagacgtgtgctcttccgatctactataccagtccacggatag

SEQ ID NO: 493 - DNA, 58 bp, synthetic construct
  source 1..58: mol_type = other DNA
gtgactggagttcagacgtgtgctcttccgatctggctacttctttgatttctggaac

SEQ ID NO: 494 - DNA, 60 bp, synthetic construct
  source 1..60: mol_type = other DNA
gtgactggagttcagacgtgtgctcttccgatctattatgacttgtcacaatgtcaccac

SEQ ID NO: 495 - DNA, 57 bp, synthetic construct
  source 1..57: mol_type = other DNA
gtgactggagttcagacgtgtgctcttccgatctttttctgtttggcttgacttgac

SEQ ID NO: 496 - DNA, 59 bp, synthetic construct
  source 1..59: mol_type = other DNA
gtgactggagttcagacgtgtgctcttccgatctacccctccttcctagagagttagag

SEQ ID NO: 497 - DNA, 57 bp, synthetic construct
  source 1..57: mol_type = other DNA
gtgactggagttcagacgtgtgctcttccgatctccccaagagagcaacacccacac

SEQ ID NO: 498 - DNA, 56 bp, synthetic construct
  source 1..56: mol_type = other DNA
gtgactggagttcagacgtgtgctcttccgatctccctgctgactcctctcctgac

SEQ ID NO: 499 - DNA, 54 bp, synthetic construct
  source 1..54: mol_type = other DNA
gtgactggagttcagacgtgtgctcttccgatctacaggatgcagagacagagc

SEQ ID NO: 500 - DNA, 60 bp, synthetic construct
  source 1..60: mol_type = other DNA
gtgactggagttcagacgtgtgctcttccgatctggtggaaagaacctctcaacattgtc

SEQ ID NO: 501 - DNA, 55 bp, synthetic construct
  source 1..55: mol_type = other DNA
gtgactggagttcagacgtgtgctcttccgatcttagaaccattgctcaaaggac

SEQ ID NO: 502 - DNA, 55 bp, synthetic construct
  source 1..55: mol_type = other DNA
gtgactggagttcagacgtgtgctcttccgatctgccgaagtctgaccctttttg

SEQ ID NO: 503 - DNA, 55 bp, synthetic construct
  source 1..55: mol_type = other DNA
gtgactggagttcagacgtgtgctcttccgatcttggtccaccgccagtctcctg

SEQ ID NO: 504 - DNA, 56 bp, synthetic construct
  source 1..56: mol_type = other DNA
gtgactggagttcagacgtgtgctcttccgatcttggtagttgcatatggtgattt

SEQ ID NO: 505 - DNA, 54 bp, synthetic construct
  source 1..54: mol_type = other DNA
gtgactggagttcagacgtgtgctcttccgatctggtgccatttggtgatttcc

SEQ ID NO: 506 - DNA, 57 bp, synthetic construct
  source 1..57: mol_type = other DNA
gtgactggagttcagacgtgtgctcttccgatctatgagaagagggttgaggagttc

SEQ ID NO: 507 - DNA, 58 bp, synthetic construct
  source 1..58: mol_type = other DNA
gtgactggagttcagacgtgtgctcttccgatcttgcagttgtaggttataactatcc

SEQ ID NO: 508 - DNA, 58 bp, synthetic construct
  source 1..58: mol_type = other DNA
gtgactggagttcagacgtgtgctcttccgatctttcaatgggtcatatcacagattc

SEQ ID NO: 509 - DNA, 54 bp, synthetic construct
  source 1..54: mol_type = other DNA
gtgactggagttcagacgtgtgctcttccgatctggcagcaacagtcttacctg

SEQ ID NO: 510 - DNA, 54 bp, synthetic construct
  source 1..54: mol_type = other DNA
gtgactggagttcagacgtgtgctcttccgatctggattgcctttaccactcag

SEQ ID NO: 511 - DNA, 58 bp, synthetic construct
  source 1..58: mol_type = other DNA
gtgactggagttcagacgtgtgctcttccgatctctgcattctgactttcagtaaggc

SEQ ID NO: 512 - DNA, 60 bp, synthetic construct
  source 1..60: mol_type = other DNA
gtgactggagttcagacgtgtgctcttccgatctacatgctccataaatattctaacatg

SEQ ID NO: 513 - DNA, 60 bp, synthetic construct
  source 1..60: mol_type = other DNA
gtgactggagttcagacgtgtgctcttccgatctacttcttgtgcattgcaggtaatatc

SEQ ID NO: 514 - DNA, 54 bp, synthetic construct
  source 1..54: mol_type = other DNA
gtgactggagttcagacgtgtgctcttccgatctcctgagcgtcatctgccccc

SEQ ID NO: 515 - DNA, 55 bp, synthetic construct
  source 1..55: mol_type = other DNA
gtgactggagttcagacgtgtgctcttccgatctacccaaatcctcacgcaaccc

SEQ ID NO: 516 - DNA, 54 bp, synthetic construct
  source 1..54: mol_type = other DNA
gtgactggagttcagacgtgtgctcttccgatctcaggggcatccatgggagcc

SEQ ID NO: 517 - DNA, 60 bp, synthetic construct
  source 1..60: mol_type = other DNA
gtgactggagttcagacgtgtgctcttccgatctgctcagaacctggtatctactttctg

SEQ ID NO: 518 - DNA, 56 bp, synthetic construct
  source 1..56: mol_type = other DNA
gtgactggagttcagacgtgtgctcttccgatctcggggagaggtggagaggcttc

SEQ ID NO: 519 - DNA, 54 bp, synthetic construct
  source 1..54: mol_type = other DNA
gtgactggagttcagacgtgtgctcttccgatctactggcatgacccccacccc

SEQ ID NO: 520 - DNA, 55 bp, synthetic construct
  source 1..55: mol_type = other DNA
gtgactggagttcagacgtgtgctcttccgatctgaccgtgtccttaccgtgacg

SEQ ID NO: 521 - DNA, 55 bp, synthetic construct
  source 1..55: mol_type = other DNA
gtgactggagttcagacgtgtgctcttccgatcttcatcgaccaagtccagaatg

SEQ ID NO: 522 - DNA, 60 bp, synthetic construct
  source 1..60: mol_type = other DNA
gtgactggagttcagacgtgtgctcttccgatctagacattgttggccaaaatagtccag

SEQ ID NO: 523 - DNA, 56 bp, synthetic construct
  source 1..56: mol_type = other DNA
gtgactggagttcagacgtgtgctcttccgatctgcagcagccaccgtgagttcag

SEQ ID NO: 524 - DNA, 55 bp, synthetic construct
  source 1..55: mol_type = other DNA
gtgactggagttcagacgtgtgctcttccgatctaccctccagcgaatttcatac

SEQ ID NO: 525 - DNA, 57 bp, synthetic construct
  source 1..57: mol_type = other DNA
gtgactggagttcagacgtgtgctcttccgatctggctgttccttcaaccaccttcc

SEQ ID NO: 526 - DNA, 56 bp, synthetic construct
  source 1..56: mol_type = other DNA
gtgactggagttcagacgtgtgctcttccgatctagagcttgtttttcactggatc

SEQ ID NO: 527 - DNA, 59 bp, synthetic construct
  source 1..59: mol_type = other DNA
gtgactggagttcagacgtgtgctcttccgatctgatcctgagtcatttcttccttttc

SEQ ID NO: 528 - DNA, 54 bp, synthetic construct
  source 1..54: mol_type = other DNA
gtgactggagttcagacgtgtgctcttccgatctagcctgaccagtgagggaag

SEQ ID NO: 529 - DNA, 59 bp, synthetic construct
  source 1..59: mol_type = other DNA
gtgactggagttcagacgtgtgctcttccgatcttgacatatggccatttctgttttcc

SEQ ID NO: 530 - DNA, 54 bp, synthetic construct
  source 1..54: mol_type = other DNA
gtgactggagttcagacgtgtgctcttccgatctattgcagtccttcccctctg

SEQ ID NO: 531 - DNA, 60 bp, synthetic construct
  source 1..60: mol_type = other DNA
gtgactggagttcagacgtgtgctcttccgatctctctccagagtgctctaatgactgag

SEQ ID NO: 532 - DNA, 54 bp, synthetic construct
  source 1..54: mol_type = other DNA
gtgactggagttcagacgtgtgctcttccgatctggaaagcccctgtttcatac

SEQ ID NO: 533 - DNA, 58 bp, synthetic construct
  source 1..58: mol_type = other DNA
gtgactggagttcagacgtgtgctcttccgatctgcatgcgcttgacatcagtttgcc

SEQ ID NO: 534 - DNA, 56 bp, synthetic construct
  source 1..56: mol_type = other DNA
gtgactggagttcagacgtgtgctcttccgatctaataaaaggcagcttggacacg

SEQ ID NO: 535 - DNA, 57 bp, synthetic construct
  source 1..57: mol_type = other DNA
gtgactggagttcagacgtgtgctcttccgatctaatggttttcttttctcctccaa

SEQ ID NO: 536 - DNA, 60 bp, synthetic construct
  source 1..60: mol_type = other DNA
gtgactggagttcagacgtgtgctcttccgatctcctttgcaggactgtcaagcagagaa

SEQ ID NO: 537 - DNA, 55 bp, synthetic construct
  source 1..55: mol_type = other DNA
gtgactggagttcagacgtgtgctcttccgatctaggagagcatttaagggagag

SEQ ID NO: 538 - DNA, 60 bp, synthetic construct
  source 1..60: mol_type = other DNA
gtgactggagttcagacgtgtgctcttccgatctaaaatcatatgctccactaacaaccc

SEQ ID NO: 539 - DNA, 54 bp, synthetic construct
  source 1..54: mol_type = other DNA
gtgactggagttcagacgtgtgctcttccgatcttgggtcatgttgcagcagtc

SEQ ID NO: 540 - DNA, 59 bp, synthetic construct
  source 1..59: mol_type = other DNA
gtgactggagttcagacgtgtgctcttccgatctaagtcccaaccatgacaagattttc

SEQ ID NO: 541 - DNA, 60 bp, synthetic construct
  source 1..60: mol_type = other DNA
gtgactggagttcagacgtgtgctcttccgatctacccaaaagtaatcatcttaagtgtt

SEQ ID NO: 542 - DNA, 60 bp, synthetic construct
  source 1..60: mol_type = other DNA
gtgactggagttcagacgtgtgctcttccgatctaggaagcttagatagttctcgttctg

SEQ ID NO: 543 - DNA, 55 bp, synthetic construct
  source 1..55: mol_type = other DNA
gtgactggagttcagacgtgtgctcttccgatctcaggtcacggggagccaatgg

SEQ ID NO: 544 - DNA, 57 bp, synthetic construct
  source 1..57: mol_type = other DNA
gtgactggagttcagacgtgtgctcttccgatctcatcagctgaagatgaaatagga

SEQ ID NO: 545 - DNA, 57 bp, synthetic construct
  source 1..57: mol_type = other DNA
gtgactggagttcagacgtgtgctcttccgatctccctccaaaagtggtgctcagac

SEQ ID NO: 546 - DNA, 56 bp, synthetic construct
  source 1..56: mol_type = other DNA
gtgactggagttcagacgtgtgctcttccgatcttggacaaaccatgccaccaagc

SEQ ID NO: 547 - DNA, 59 bp, synthetic construct
  source 1..59: mol_type = other DNA
gtgactggagttcagacgtgtgctcttccgatctggtgccctactacctggagaacgag

SEQ ID NO: 548 - DNA, 55 bp, synthetic construct
  source 1..55: mol_type = other DNA
gtgactggagttcagacgtgtgctcttccgatctctgcaaggccttcttcaagag

SEQ ID NO: 549 - DNA, 57 bp, synthetic construct
  source 1..57: mol_type = other DNA
gtgactggagttcagacgtgtgctcttccgatctagctgccaggcctgccggctccg

SEQ ID NO: 550 - DNA, 58 bp, synthetic construct
  source 1..58: mol_type = other DNA
gtgactggagttcagacgtgtgctcttccgatctgtataaaagtttacacgggaaaaa

SEQ ID NO: 551 - DNA, 55 bp, synthetic construct
  source 1..55: mol_type = other DNA
gtgactggagttcagacgtgtgctcttccgatctctgctggagacatgagagctg

SEQ ID NO: 552 - DNA, 54 bp, synthetic construct
  source 1..54: mol_type = other DNA
gtgactggagttcagacgtgtgctcttccgatcttttgatcatgagcgggcttg

SEQ ID NO: 553 - DNA, 54 bp, synthetic construct
  source 1..54: mol_type = other DNA
gtgactggagttcagacgtgtgctcttccgatcttgccttgttggatgctgagc

SEQ ID NO: 554 - DNA, 60 bp, synthetic construct
  source 1..60: mol_type = other DNA
gtgactggagttcagacgtgtgctcttccgatctcactgaagggtctggtaggatcatac

SEQ ID NO: 555 - DNA, 54 bp, synthetic construct
  source 1..54: mol_type = other DNA
gtgactggagttcagacgtgtgctcttccgatctatgatcaactgggcgaagag

SEQ ID NO: 556 - DNA, 60 bp, synthetic construct
  source 1..60: mol_type = other DNA
gtgactggagttcagacgtgtgctcttccgatctacaagttcttgaaaagctattgactc

SEQ ID NO: 557 - DNA, 54 bp, synthetic construct
  source 1..54: mol_type = other DNA
gtgactggagttcagacgtgtgctcttccgatctggttggctctaaagtagtcc

SEQ ID NO: 558 - DNA, 54 bp, synthetic construct
  source 1..54: mol_type = other DNA
gtgactggagttcagacgtgtgctcttccgatctggattatctgaaccgtgtgg

SEQ ID NO: 559 - DNA, 60 bp, synthetic construct
  source 1..60: mol_type = other DNA
gtgactggagttcagacgtgtgctcttccgatctgtatatacaatgccacctgaatacag

SEQ ID NO: 560 - DNA, 54 bp, synthetic construct
  source 1..54: mol_type = other DNA
gtgactggagttcagacgtgtgctcttccgatcttgtccccagtccattttcac

SEQ ID NO: 561 - DNA, 55 bp, synthetic construct
  source 1..55: mol_type = other DNA
gtgactggagttcagacgtgtgctcttccgatctgaccgcatcgtggccttgacc

SEQ ID NO: 562 - DNA, 54 bp, synthetic construct
  source 1..54: mol_type = other DNA
gtgactggagttcagacgtgtgctcttccgatcttcagatgaaaccaccagcac

SEQ ID NO: 563 - DNA, 54 bp, synthetic construct
  source 1..54: mol_type = other DNA
gtgactggagttcagacgtgtgctcttccgatctccgcctcccgctctcccttc

SEQ ID NO: 564 - DNA, 55 bp, synthetic construct
  source 1..55: mol_type = other DNA
gtgactggagttcagacgtgtgctcttccgatctcccaggggagccttcaggttc

SEQ ID NO: 565 - DNA, 55 bp, synthetic construct
  source 1..55: mol_type = other DNA
gtgactggagttcagacgtgtgctcttccgatctgaagccaaacctgctcacctg

SEQ ID NO: 566 - DNA, 55 bp, synthetic construct
  source 1..55: mol_type = other DNA
gtgactggagttcagacgtgtgctcttccgatctgataccccagctcagatcttc

SEQ ID NO: 567 - DNA, 57 bp, synthetic construct
  source 1..57: mol_type = other DNA
gtgactggagttcagacgtgtgctcttccgatctagtaaatgagtctcaacgtgttc

SEQ ID NO: 568 - DNA, 55 bp, synthetic construct
  source 1..55: mol_type = other DNA
gtgactggagttcagacgtgtgctcttccgatctgttcatggatgcactggagtc

SEQ ID NO: 569 - DNA, 58 bp, synthetic construct
  source 1..58: mol_type = other DNA
gtgactggagttcagacgtgtgctcttccgatctggtacaggggcgaggtcatcactg

SEQ ID NO: 570 - DNA, 60 bp, synthetic construct
  source 1..60: mol_type = other DNA
gtgactggagttcagacgtgtgctcttccgatcttccatctctttgtcggtggtattaac

SEQ ID NO: 571 - DNA, 60 bp, synthetic construct
  source 1..60: mol_type = other DNA
gtgactggagttcagacgtgtgctcttccgatctccacagtgagctcgatcctccttttc

SEQ ID NO: 572 - DNA, 59 bp, synthetic construct
  source 1..59: mol_type = other DNA
gtgactggagttcagacgtgtgctcttccgatctatggacaacagtcaaacaacaattc

SEQ ID NO: 573 - DNA, 58 bp, synthetic construct
  source 1..58: mol_type = other DNA
gtgactggagttcagacgtgtgctcttccgatctgcattagaaagcctgtagttttac

SEQ ID NO: 574 - DNA, 60 bp, synthetic construct
  source 1..60: mol_type = other DNA
gtgactggagttcagacgtgtgctcttccgatcttacggtagtgggggaaggcatatatc

SEQ ID NO: 575 - DNA, 56 bp, synthetic construct
  source 1..56: mol_type = other DNA
gtgactggagttcagacgtgtgctcttccgatctcaggtttctaacgcctgttttc

SEQ ID NO: 576 - DNA, 57 bp, synthetic construct
  source 1..57: mol_type = other DNA
gtgactggagttcagacgtgtgctcttccgatctagggtacaaattctcagatcatc

SEQ ID NO: 577 - DNA, 56 bp, synthetic construct
  source 1..56: mol_type = other DNA
gtgactggagttcagacgtgtgctcttccgatctcgcgatggcccagctcctcagc

SEQ ID NO: 578 - DNA, 54 bp, synthetic construct
  source 1..54: mol_type = other DNA
gtgactggagttcagacgtgtgctcttccgatctgggcttcctggacacgctgg

SEQ ID NO: 579 - DNA, 59 bp, synthetic construct
  source 1..59: mol_type = other DNA
gtgactggagttcagacgtgtgctcttccgatctctacacaagcttcctttccgtcatg

SEQ ID NO: 580 - DNA, 54 bp, synthetic construct
  source 1..54: mol_type = other DNA
gtgactggagttcagacgtgtgctcttccgatctaacttcgtcctccagagtcg

SEQ ID NO: 581 - RNA, 54 nt, synthetic construct
  source 1..54: mol_type = other RNA
gtgactggagttcagacgtgtgctcttccgatctcggagagggggagagcaggc

SEQ ID NO: 582 - DNA, 58 bp, synthetic construct
  source 1..58: mol_type = other DNA
gtgactggagttcagacgtgtgctcttccgatctatccattgcctgtctaaagaacac

SEQ ID NO: 583 - DNA, 60 bp, synthetic construct
  source 1..60: mol_type = other DNA
gtgactggagttcagacgtgtgctcttccgatcttgttattaatatgagtattgttaacc

SEQ ID NO: 584 - DNA, 54 bp, synthetic construct
  source 1..54: mol_type = other DNA
gtgactggagttcagacgtgtgctcttccgatcttgaaggctgttccctgtttc

SEQ ID NO: 585 - DNA, 58 bp, synthetic construct
  source 1..58: mol_type = other DNA
gtgactggagttcagacgtgtgctcttccgatctgcgcaacaagcccactgtctatgg

SEQ ID NO: 586 - DNA, 55 bp, synthetic construct
  source 1..55: mol_type = other DNA
gtgactggagttcagacgtgtgctcttccgatctatctcccacttgtcgtagttg

SEQ ID NO: 587 - DNA, 54 bp, synthetic construct
  source 1..54: mol_type = other DNA
gtgactggagttcagacgtgtgctcttccgatctagacgccttgccccacgtac

SEQ ID NO: 588 - DNA, 60 bp, synthetic construct
  source 1..60: mol_type = other DNA
gtgactggagttcagacgtgtgctcttccgatctaaagcacttcctgaaataatttcacc

SEQ ID NO: 589 - DNA, 54 bp, synthetic construct
  source 1..54: mol_type = other DNA
gtgactggagttcagacgtgtgctcttccgatctcgagaccctctcttcagagc

SEQ ID NO: 590 - DNA, 59 bp, synthetic construct
  source 1..59: mol_type = other DNA
gtgactggagttcagacgtgtgctcttccgatcttgttgaagtcctcgttgtcttgttg

SEQ ID NO: 591 - DNA, 55 bp, synthetic construct
  source 1..55: mol_type = other DNA
gtgactggagttcagacgtgtgctcttccgatctggctggggctttttgtaaaag

SEQ ID NO: 592 - DNA, 60 bp, synthetic construct
  source 1..60: mol_type = other DNA
gtgactggagttcagacgtgtgctcttccgatctatgcaacatttctaaagttacctact

SEQ ID NO: 593 - DNA, 55 bp, synthetic construct
  source 1..55: mol_type = other DNA
gtgactggagttcagacgtgtgctcttccgatctacaatcatgttgcagcaattc

SEQ ID NO: 594 - DNA, 60 bp, synthetic construct
  source 1..60: mol_type = other DNA
gtgactggagttcagacgtgtgctcttccgatcttatcattacaccagttcgtccctttc

SEQ ID NO: 595 - DNA, 54 bp, synthetic construct
  source 1..54: mol_type = other DNA
gtgactggagttcagacgtgtgctcttccgatcttcagatccaggaagaggaaa

SEQ ID NO: 596 - DNA, 54 bp, synthetic construct
  source 1..54: mol_type = other DNA
gtgactggagttcagacgtgtgctcttccgatctctacgacccagttaccatag

SEQ ID NO: 597 - DNA, 58 bp, synthetic construct
  source 1..58: mol_type = other DNA
gtgactggagttcagacgtgtgctcttccgatctacatggaaggatgagaatttcaag

SEQ ID NO: 598 - DNA, 60 bp, synthetic construct
  source 1..60: mol_type = other DNA
gtgactggagttcagacgtgtgctcttccgatctttcctgtgaaataatactggtatgta

SEQ ID NO: 599 - DNA, 60 bp, synthetic construct
  source 1..60: mol_type = other DNA
gtgactggagttcagacgtgtgctcttccgatcttcttcataccaggaccagaggaaacc

SEQ ID NO: 600 - DNA, 60 bp, synthetic construct
  source 1..60: mol_type = other DNA
gtgactggagttcagacgtgtgctcttccgatcttttgtctttatttgctttgtcaagat

SEQ ID NO: 601 - DNA, 60 bp, synthetic construct
  source 1..60: mol_type = other DNA
gtgactggagttcagacgtgtgctcttccgatcttcagctgtactcctagaattaaacac

SEQ ID NO: 602 - DNA, 60 bp, synthetic construct
  source 1..60: mol_type = other DNA
gtgactggagttcagacgtgtgctcttccgatctgttggtagaagacttggatcgaattc

SEQ ID NO: 603 - DNA, 55 bp, synthetic construct
  source 1..55: mol_type = other DNA
gtgactggagttcagacgtgtgctcttccgatctttgggcgaatgcagtttttcc

SEQ ID NO: 604 - DNA, 60 bp, synthetic construct
  source 1..60: mol_type = other DNA
gtgactggagttcagacgtgtgctcttccgatctctgatttgtgaatatgcctactgttc

SEQ ID NO: 605 - DNA, 54 bp, synthetic construct
  source 1..54: mol_type = other DNA
gtgactggagttcagacgtgtgctcttccgatcttggagttcatggaggagctg

SEQ ID NO: 606 - DNA, 60 bp, synthetic construct
  source 1..60: mol_type = other DNA
gtgactggagttcagacgtgtgctcttccgatctaggtagtctggggaagctgtaatctc

SEQ ID NO: 607 - DNA, 55 bp, synthetic construct
  source 1..55: mol_type = other DNA
gtgactggagttcagacgtgtgctcttccgatctctctttgtccgtggtgttaac

SEQ ID NO: 608 - DNA, 58 bp, synthetic construct
  source 1..58: mol_type = other DNA
gtgactggagttcagacgtgtgctcttccgatctgtggccaaacccatgaaggagacc

SEQ ID NO: 609 - DNA, 58 bp, synthetic construct
  source 1..58: mol_type = other DNA
gtgactggagttcagacgtgtgctcttccgatctgtcactgacagccctctggacaac

SEQ ID NO: 610 - DNA, 57 bp, synthetic construct
  source 1..57: mol_type = other DNA
gtgactggagttcagacgtgtgctcttccgatctggactacgcgcaatgccttcagc

SEQ ID NO: 611 - DNA, 54 bp, synthetic construct
  source 1..54: mol_type = other DNA
gtgactggagttcagacgtgtgctcttccgatctctcccgggccagcctcacgg

SEQ ID NO: 612 - DNA, 55 bp, synthetic construct
  source 1..55: mol_type = other DNA
gtgactggagttcagacgtgtgctcttccgatctacggaaggtcctgagggggtc

SEQ ID NO: 613 - DNA, 54 bp, synthetic construct
  source 1..54: mol_type = other DNA
gtgactggagttcagacgtgtgctcttccgatctcagctgctggcacctggacg

SEQ ID NO: 614 - DNA, 57 bp, synthetic construct
  source 1..57: mol_type = other DNA
gtgactggagttcagacgtgtgctcttccgatctaggagaccctgtaggaggacccc

SEQ ID NO: 615 - DNA, 60 bp, synthetic construct
  source 1..60: mol_type = other DNA
gtgactggagttcagacgtgtgctcttccgatctctcattaaaagaggtgttcttgtgac

SEQ ID NO: 616 - DNA, 59 bp, synthetic construct
  source 1..59: mol_type = other DNA
gtgactggagttcagacgtgtgctcttccgatctaaaatcacatggcatctgtacagtg

SEQ ID NO: 617 - DNA, 54 bp, synthetic construct
  source 1..54: mol_type = other DNA
gtgactggagttcagacgtgtgctcttccgatcttgagacatgtctgagactcc

SEQ ID NO: 618 - DNA, 59 bp, synthetic construct
  source 1..59: mol_type = other DNA
gtgactggagttcagacgtgtgctcttccgatctactgggctctctggaggtgtatatg

SEQ ID NO: 619 - DNA, 55 bp, synthetic construct
  source 1..55: mol_type = other DNA
gtgactggagttcagacgtgtgctcttccgatctcgccacttcatgttccagtgc

SEQ ID NO: 620 - DNA, 57 bp, synthetic construct
  source 1..57: mol_type = other DNA
gtgactggagttcagacgtgtgctcttccgatctactgggctacgtcatccctgctc

SEQ ID NO: 621 - DNA, 60 bp, synthetic construct
  source 1..60: mol_type = other DNA
gtgactggagttcagacgtgtgctcttccgatctcaaagaggactttcgctatcgatctc

SEQ ID NO: 622 - DNA, 54 bp, synthetic construct
  source 1..54: mol_type = other DNA
gtgactggagttcagacgtgtgctcttccgatctcggaggtcatactgcatcag

SEQ ID NO: 623 - DNA, 55 bp, synthetic construct
  source 1..55: mol_type = other DNA
gtgactggagttcagacgtgtgctcttccgatctgatggcctgggggtcctctgg

SEQ ID NO: 624 - DNA, 55 bp, synthetic construct
  source 1..55: mol_type = other DNA
gtgactggagttcagacgtgtgctcttccgatctccgcctgaagagcacgccatg

SEQ ID NO: 625 - DNA, 60 bp, synthetic construct
  source 1..60: mol_type = other DNA
gtgactggagttcagacgtgtgctcttccgatctgcaactgatagacgtaatcccaaagc

SEQ ID NO: 626 - DNA, 56 bp, synthetic construct
  source 1..56: mol_type = other DNA
gtgactggagttcagacgtgtgctcttccgatctgatcccaacggactggctcgac

SEQ ID NO: 627 - DNA, 58 bp, synthetic construct
  source 1..58: mol_type = other DNA
gtgactggagttcagacgtgtgctcttccgatctctggacattttctcataggtcatg

SEQ ID NO: 628 - DNA, 60 bp, synthetic construct
  source 1..60: mol_type = other DNA
gtgactggagttcagacgtgtgctcttccgatctctctcatttacataatctgctctgtc

SEQ ID NO: 629 - DNA, 57 bp, synthetic construct
  source 1..57: mol_type = other DNA
gtgactggagttcagacgtgtgctcttccgatctggccagaaggaaccatcgggaac

SEQ ID NO: 630 - DNA, 60 bp, synthetic construct
  source 1..60: mol_type = other DNA
gtgactggagttcagacgtgtgctcttccgatctttcagcagaaagaggactcagaatag

SEQ ID NO: 631 - DNA, 56 bp, synthetic construct
  source 1..56: mol_type = other DNA
gtgactggagttcagacgtgtgctcttccgatctctaccttatatcccttctcacc

SEQ ID NO: 632 - DNA, 54 bp, synthetic construct
  source 1..54: mol_type = other DNA
gtgactggagttcagacgtgtgctcttccgatctaggtagtccagggtatgtgg

SEQ ID NO: 633 - DNA, 57 bp, synthetic construct
  source 1..57: mol_type = other DNA
gtgactggagttcagacgtgtgctcttccgatctccccatccattcttcctattccc

SEQ ID NO: 634 - DNA, 54 bp, synthetic construct
  source 1..54: mol_type = other DNA
gtgactggagttcagacgtgtgctcttccgatctcccaaccctccaccaccttc

SEQ ID NO: 635 - DNA, 55 bp, synthetic construct
  source 1..55: mol_type = other DNA
gtgactggagttcagacgtgtgctcttccgatctacgaactgtgctgatgggaag

SEQ ID NO: 636 - DNA, 60 bp, synthetic construct
  source 1..60: mol_type = other DNA
gtgactggagttcagacgtgtgctcttccgatctggtgcctatgggacagtgtacaaggc

SEQ ID NO: 637 - DNA, 55 bp, synthetic construct
  source 1..55: mol_type = other DNA
gtgactggagttcagacgtgtgctcttccgatctggtgataggagtctgtgattg

SEQ ID NO: 638 - DNA, 58 bp, synthetic construct
  source 1..58: mol_type = other DNA
gtgactggagttcagacgtgtgctcttccgatctcccttgcctccctttccaatggac

SEQ ID NO: 639 - DNA, 60 bp, synthetic construct
  source 1..60: mol_type = other DNA
gtgactggagttcagacgtgtgctcttccgatctacaagggagcagcagactttgtggtc

SEQ ID NO: 640 - DNA, 60 bp, synthetic construct
  source 1..60: mol_type = other DNA
gtgactggagttcagacgtgtgctcttccgatctgtctctgagtccactaaaagttgtgc

SEQ ID NO: 641 - DNA, 54 bp, synthetic construct
  source 1..54: mol_type = other DNA
gtgactggagttcagacgtgtgctcttccgatctagcgtatccaagaggcctag

SEQ ID NO: 642 - DNA, 56 bp, synthetic construct
  source 1..56: mol_type = other DNA
gtgactggagttcagacgtgtgctcttccgatctgtggccatctggatgcgtgcac

SEQ ID NO: 643 - DNA, 55 bp, synthetic construct
  source 1..55: mol_type = other DNA
gtgactggagttcagacgtgtgctcttccgatctttagaatcatcctggcttctg

SEQ ID NO: 644 - DNA, 56 bp, synthetic construct
  source 1..56: mol_type = other DNA
gtgactggagttcagacgtgtgctcttccgatctccatcccaggagcttacttccc

SEQ ID NO: 645 - DNA, 57 bp, synthetic construct
  source 1..57: mol_type = other DNA
gtgactggagttcagacgtgtgctcttccgatctttggctttatgcttattttgtcc

SEQ ID NO: 646 - DNA, 58 bp, synthetic construct
  source 1..58: mol_type = other DNA
gtgactggagttcagacgtgtgctcttccgatctacacaacacaaaatagccgtataa

SEQ ID NO: 647 - DNA, 57 bp, synthetic construct
  source 1..57: mol_type = other DNA
gtgactggagttcagacgtgtgctcttccgatctcatcacagtaaataacactctgg

SEQ ID NO: 648 - DNA, 57 bp, synthetic construct
  source 1..57: mol_type = other DNA
gtgactggagttcagacgtgtgctcttccgatctcccccaaatctgaatcccgagag

SEQ ID NO: 649 - DNA, 55 bp, synthetic construct
  source 1..55: mol_type = other DNA
gtgactggagttcagacgtgtgctcttccgatctctctcaccacccgcacgtctg

SEQ ID NO: 650 - DNA, 57 bp, synthetic construct
  source 1..57: mol_type = other DNA
gtgactggagttcagacgtgtgctcttccgatctccaggtcagtggatcccctctcc

SEQ ID NO: 651 - DNA, 54 bp, synthetic construct
  source 1..54: mol_type = other DNA
gtgactggagttcagacgtgtgctcttccgatctttgaaagatggcggctgcag

SEQ ID NO: 652 - DNA, 54 bp, synthetic construct
  source 1..54: mol_type = other DNA
gtgactggagttcagacgtgtgctcttccgatctggccaacttgttcagtggag

SEQ ID NO: 653 - DNA, 55 bp, synthetic construct
  source 1..55: mol_type = other DNA
gtgactggagttcagacgtgtgctcttccgatctgtcatctctcctccctgcttc

SEQ ID NO: 654 - DNA, 54 bp, synthetic construct
  source 1..54: mol_type = other DNA
gtgactggagttcagacgtgtgctcttccgatctctttgaccatgaaggcagga

SEQ ID NO: 655 - DNA, 60 bp, synthetic construct
  source 1..60: mol_type = other DNA
gtgactggagttcagacgtgtgctcttccgatctggtactgtgtatatacttacttctcc

SEQ ID NO: 656 - DNA, 60 bp, synthetic construct
  source 1..60: mol_type = other DNA
gtgactggagttcagacgtgtgctcttccgatctggcattttgagtgttagactggaaac

SEQ ID NO: 657 - DNA, 60 bp, synthetic construct
  source 1..60: mol_type = other DNA
gtgactggagttcagacgtgtgctcttccgatctattcacttttatcacctttccttgcc

SEQ ID NO: 658 - DNA, 54 bp, synthetic construct
  source 1..54: mol_type = other DNA
gtgactggagttcagacgtgtgctcttccgatctccgcttcttgtcctgcttgc

SEQ ID NO: 659 - DNA, 60 bp, synthetic construct
  source 1..60: mol_type = other DNA
gtgactggagttcagacgtgtgctcttccgatcttgcctcttgcttctcttttcctatcc

SEQ ID NO: 660 - DNA, 54 bp, synthetic construct
  source 1..54: mol_type = other DNA
gtgactggagttcagacgtgtgctcttccgatctgcccaggggtcagaggcaag

SEQ ID NO: 661 - DNA, 58 bp, synthetic construct
  source 1..58: mol_type = other DNA
gtgactggagttcagacgtgtgctcttccgatctgcctcatcttgggcctgtgttatc

SEQ ID NO: 662 - DNA, 54 bp, synthetic construct
  source 1..54: mol_type = other DNA
gtgactggagttcagacgtgtgctcttccgatctctcccagagaccccagttgc

SEQ ID NO: 663 - DNA, 55 bp, synthetic construct
  source 1..55: mol_type = other DNA
gtgactggagttcagacgtgtgctcttccgatctcccaggcctctgattcctcac

SEQ ID NO: 664 - DNA, 55 bp, synthetic construct
  source 1..55: mol_type = other DNA
gtgactggagttcagacgtgtgctcttccgatcttgtcgtctctccagccccagc

SEQ ID NO: 665 - DNA, 59 bp, synthetic construct
  source 1..59: mol_type = other DNA
gtgactggagttcagacgtgtgctcttccgatctgactttcaactctgtctccttcctc

SEQ ID NO: 666 - DNA, 54 bp, synthetic construct
  source 1..54: mol_type = other DNA
gtgactggagttcagacgtgtgctcttccgatctcaggcattgaagtctcatgg

SEQ ID NO: 667 - DNA, 57 bp, synthetic construct
  source 1..57: mol_type = other DNA
gtgactggagttcagacgtgtgctcttccgatcttctgggaagggacagaagatgac

SEQ ID NO: 668 - DNA, 54 bp, synthetic construct
  source 1..54: mol_type = other DNA
gtgactggagttcagacgtgtgctcttccgatctagctcccagaatgccagagg

SEQ ID NO: 669 - DNA, 55 bp, synthetic construct
  source 1..55: mol_type = other DNA
gtgactggagttcagacgtgtgctcttccgatcttgactgctcttttcacccatc

SEQ ID NO: 670 - DNA, 54 bp, synthetic construct
  source 1..54: mol_type = other DNA
gtgactggagttcagacgtgtgctcttccgatctccccagcccaacccttgtcc

SEQ ID NO: 671 - DNA, 55 bp, synthetic construct
  source 1..55: mol_type = other DNA
gtgactggagttcagacgtgtgctcttccgatctaattccatgggactgactttc

SEQ ID NO: 672 - DNA, 54 bp, synthetic construct
  source 1..54: mol_type = other DNA
gtgactggagttcagacgtgtgctcttccgatcttgctagggggctggggttgg

SEQ ID NO: 673 - DNA, 55 bp, synthetic construct
  source 1..55: mol_type = other DNA
gtgactggagttcagacgtgtgctcttccgatctcatgctggatccccacttttc

SEQ ID NO: 674 - DNA, 60 bp, synthetic construct
  source 1..60: mol_type = other DNA
gtgactggagttcagacgtgtgctcttccgatctgtgcaagtgaaagccttatatctttc

SEQ ID NO: 675 - DNA, 58 bp, synthetic construct
  source 1..58: mol_type = other DNA
gtgactggagttcagacgtgtgctcttccgatctacatgggaaaacataaccttgaat

SEQ ID NO: 676 - DNA, 54 bp, synthetic construct
  source 1..54: mol_type = other DNA
gtgactggagttcagacgtgtgctcttccgatctggctgagtcctggcgctgtg

SEQ ID NO: 677 - DNA, 55 bp, synthetic construct
  source 1..55: mol_type = other DNA
gtgactggagttcagacgtgtgctcttccgatctccccaacgctgcccctgctca

SEQ ID NO: 678 - DNA, 59 bp, synthetic construct
  source 1..59: mol_type = other DNA
gtgactggagttcagacgtgtgctcttccgatctggctttggtgagatccattgacctc

SEQ ID NO: 679 - DNA, 57 bp, synthetic construct
  source 1..57: mol_type = other DNA
gtgactggagttcagacgtgtgctcttccgatcttgtcaagaaaccatgatctctgt

SEQ ID NO: 680 - DNA, 55 bp, synthetic construct
  source 1..55: mol_type = other DNA
gtgactggagttcagacgtgtgctcttccgatcttaaccagctgtcctcctcccc

SEQ ID NO: 681 - DNA, 54 bp, synthetic construct
  source 1..54: mol_type = other DNA
gtgactggagttcagacgtgtgctcttccgatctaacaacacagaagcaaagcg

SEQ ID NO: 682 - DNA, 60 bp, synthetic construct
  source 1..60: mol_type = other DNA
gtgactggagttcagacgtgtgctcttccgatctgaaccaagttcttccgagggaattcc

SEQ ID NO: 683 - DNA, 55 bp, synthetic construct
  source 1..55: mol_type = other DNA
gtgactggagttcagacgtgtgctcttccgatctggaaatccttccgggcagcca

SEQ ID NO: 684 - DNA, 59 bp, synthetic construct
  source 1..59: mol_type = other DNA
gtgactggagttcagacgtgtgctcttccgatctaaatatggtcgacggtccaaatttg

SEQ ID NO: 685 - DNA, 54 bp, synthetic construct
  source 1..54: mol_type = other DNA
gtgactggagttcagacgtgtgctcttccgatctggacacaactgccacaagcc

SEQ ID NO: 686 - DNA, 57 bp, synthetic construct
  source 1..57: mol_type = other DNA
gtgactggagttcagacgtgtgctcttccgatctcctcagcccgaccaaggacaaga

SEQ ID NO: 687 - DNA, 54 bp, synthetic construct
  source 1..54: mol_type = other DNA
gtgactggagttcagacgtgtgctcttccgatcttctgcctcatgctctctcca

SEQ ID NO: 688 - DNA, 54 bp, synthetic construct
  source 1..54: mol_type = other DNA
gtgactggagttcagacgtgtgctcttccgatcttcaagtgggctgaaaatgag

SEQ ID NO: 689 - DNA, 58 bp, synthetic construct
  source 1..58: mol_type = other DNA
gtgactggagttcagacgtgtgctcttccgatctgcagcaggagagctttgccattca

SEQ ID NO: 690 - DNA, 54 bp, synthetic construct
  source 1..54: mol_type = other DNA
gtgactggagttcagacgtgtgctcttccgatctcatatgccaagagtccagac

SEQ ID NO: 691 - DNA, 54 bp, synthetic construct
  source 1..54: mol_type = other DNA
gtgactggagttcagacgtgtgctcttccgatcttgagggcctccttctcatca

SEQ ID NO: 692 - DNA, 55 bp, synthetic construct
  source 1..55: mol_type = other DNA
gtgactggagttcagacgtgtgctcttccgatctgcctccaccaccttcccaaag

SEQ ID NO: 693 - DNA, 60 bp, synthetic construct
  source 1..60: mol_type = other DNA
gtgactggagttcagacgtgtgctcttccgatctctctcgatgatcttccagcggacctg

SEQ ID NO: 694 - DNA, 54 bp, synthetic construct
  source 1..54: mol_type = other DNA
gtgactggagttcagacgtgtgctcttccgatctacatgcaggcgaccaccact

SEQ ID NO: 695 - DNA, 57 bp, synthetic construct
  source 1..57: mol_type = other DNA
gtgactggagttcagacgtgtgctcttccgatctcttgtggaagggctcctggctca

SEQ ID NO: 696 - DNA, 55 bp, synthetic construct
  source 1..55: mol_type = other DNA
gtgactggagttcagacgtgtgctcttccgatctgccgttgatgaatgtccatat

SEQ ID NO: 697 - DNA, 54 bp, synthetic construct
  source 1..54: mol_type = other DNA
gtgactggagttcagacgtgtgctcttccgatctgatccagaggaggaagtcag

SEQ ID NO: 698 - DNA, 55 bp, synthetic construct
  source 1..55: mol_type = other DNA
gtgactggagttcagacgtgtgctcttccgatctcctctcccaggggtttgccta

SEQ ID NO: 699 - DNA, 54 bp, synthetic construct
  source 1..54: mol_type = other DNA
gtgactggagttcagacgtgtgctcttccgatctttgcaggatgggccggtgag

SEQ ID NO: 700 - DNA, 55 bp, synthetic construct
  source 1..55: mol_type = other DNA
gtgactggagttcagacgtgtgctcttccgatctgtccattatgatgctccaggt

SEQ ID NO: 701 - DNA, 60 bp, synthetic construct
  source 1..60: mol_type = other DNA
gtgactggagttcagacgtgtgctcttccgatctatcttttctggggatgtccaatatgg

SEQ ID NO: 702 - DNA, 59 bp, synthetic construct
  source 1..59: mol_type = other DNA
gtgactggagttcagacgtgtgctcttccgatctctctgaagaggagtcatcatcatca

SEQ ID NO: 703 - DNA, 54 bp, synthetic construct
  source 1..54: mol_type = other DNA
gtgactggagttcagacgtgtgctcttccgatctccacttccacaggggctccc

SEQ ID NO: 704 - DNA, 54 bp, synthetic construct
  source 1..54: mol_type = other DNA
gtgactggagttcagacgtgtgctcttccgatctacgagtgcttcatcaaggtg

SEQ ID NO: 705 - DNA, 55 bp, synthetic construct
  source 1..55: mol_type = other DNA
gtgactggagttcagacgtgtgctcttccgatctgagttgttgaggaagccagac

SEQ ID NO: 706 - DNA, 54 bp, synthetic construct
  source 1..54: mol_type = other DNA
gtgactggagttcagacgtgtgctcttccgatctcccactgtgttactgccatc

SEQ ID NO: 707 - DNA, 60 bp, synthetic construct
  source 1..60: mol_type = other DNA
gtgactggagttcagacgtgtgctcttccgatcttgttctggcggtgttttgaaattagt

SEQ ID NO: 708 - DNA, 57 bp, synthetic construct
  source 1..57: mol_type = other DNA
gtgactggagttcagacgtgtgctcttccgatctgcatcaaagcagaggacaagatc

SEQ ID NO: 709 - DNA, 60 bp, synthetic construct
  source 1..60: mol_type = other DNA
gtgactggagttcagacgtgtgctcttccgatctgcttcttggtcgtgttcttcattcgg

SEQ ID NO: 710 - DNA, 59 bp, synthetic construct
  source 1..59: mol_type = other DNA
gtgactggagttcagacgtgtgctcttccgatctgcataggggtcttcttaatcgcctg

SEQ ID NO: 711 - DNA, 60 bp, synthetic construct
  source 1..60: mol_type = other DNA
gtgactggagttcagacgtgtgctcttccgatctggcctgcttatctgttcctcctcctg

SEQ ID NO: 712 - DNA, 60 bp, synthetic construct
  source 1..60: mol_type = other DNA
gtgactggagttcagacgtgtgctcttccgatctgatgtgctgggtccttttcagttgcg

SEQ ID NO: 713 - DNA, 58 bp, synthetic construct
  source 1..58: mol_type = other DNA
gtgactggagttcagacgtgtgctcttccgatctaacgagaagcacatcaacctgagc

SEQ ID NO: 714 - DNA, 60 bp, synthetic construct
  source 1..60: mol_type = other DNA
gtgactggagttcagacgtgtgctcttccgatctaaaccaaacaacttccacctctaaag

SEQ ID NO: 715 - DNA, 60 bp, synthetic construct
  source 1..60: mol_type = other DNA
gtgactggagttcagacgtgtgctcttccgatctcggaagagatgtctaatattaacttg

SEQ ID NO: 716 - DNA, 56 bp, synthetic construct
  source 1..56: mol_type = other DNA
gtgactggagttcagacgtgtgctcttccgatctaggaaacacaattagcctggac

SEQ ID NO: 717 - DNA, 55 bp, synthetic construct
  source 1..55: mol_type = other DNA
gtgactggagttcagacgtgtgctcttccgatctgagctccaaaccaagtgttag

SEQ ID NO: 718 - DNA, 60 bp, synthetic construct
  source 1..60: mol_type = other DNA
gtgactggagttcagacgtgtgctcttccgatctgaggggtgcagcattggtgaacattc

SEQ ID NO: 719 - DNA, 59 bp, synthetic construct
  source 1..59: mol_type = other DNA
gtgactggagttcagacgtgtgctcttccgatctcacattctaaagtgttcaaattcct

SEQ ID NO: 720 - DNA, 59 bp, synthetic construct
  source 1..59: mol_type = other DNA
gtgactggagttcagacgtgtgctcttccgatctacctccatgtgggtttataaatgtg

SEQ ID NO: 721 - DNA, 57 bp, synthetic construct
  source 1..57: mol_type = other DNA
gtgactggagttcagacgtgtgctcttccgatctggggaactgtaacgctgcctatc

SEQ ID NO: 722 - DNA, 56 bp, synthetic construct
  source 1..56: mol_type = other DNA
gtgactggagttcagacgtgtgctcttccgatctagcctcttcaccccgtctgctg

SEQ ID NO: 723 - DNA, 53 bp, synthetic construct
  source 1..53: mol_type = other DNA
gtgactggagttcagacgtgtgctcttccgatctcaccctaacggcccgtgca

SEQ ID NO: 724 - DNA, 55 bp, synthetic construct
  source 1..55: mol_type = other DNA
gtgactggagttcagacgtgtgctcttccgatctctctccacgctccctccactc

SEQ ID NO: 725 - DNA, 57 bp, synthetic construct
  source 1..57: mol_type = other DNA
gtgactggagttcagacgtgtgctcttccgatctcctgagagtggagctggagagac

SEQ ID NO: 726 - DNA, 54 bp, synthetic construct
  source 1..54: mol_type = other DNA
gtgactggagttcagacgtgtgctcttccgatctgtgtcctgaggcacccaagc

SEQ ID NO: 727 - DNA, 54 bp, synthetic construct
  source 1..54: mol_type = other DNA
gtgactggagttcagacgtgtgctcttccgatctcccatccagttctgccccat

SEQ ID NO: 728 - DNA, 57 bp, synthetic construct
  source 1..57: mol_type = other DNA
gtgactggagttcagacgtgtgctcttccgatctgaggacctgtgggactctgcact

SEQ ID NO: 729 - DNA, 53 bp, synthetic construct
  source 1..53: mol_type = other DNA
gtgactggagttcagacgtgtgctcttccgatctcagtgaccgccggcaggac

SEQ ID NO: 730 - DNA, 59 bp, synthetic construct
  source 1..59: mol_type = other DNA
gtgactggagttcagacgtgtgctcttccgatcttagctccttgtcggtggtgttagcg

SEQ ID NO: 731 - DNA, 53 bp, synthetic construct
  source 1..53: mol_type = other DNA
gtgactggagttcagacgtgtgctcttccgatcttcgtcagcctccaccagct

SEQ ID NO: 732 - DNA, 56 bp, synthetic construct
  source 1..56: mol_type = other DNA
gtgactggagttcagacgtgtgctcttccgatctgctcatggacgcgttggactcc

SEQ ID NO: 733 - DNA, 56 bp, synthetic construct
  source 1..56: mol_type = other DNA
gtgactggagttcagacgtgtgctcttccgatctgggacccaagctggacttccca

SEQ ID NO: 734 - DNA, 59 bp, synthetic construct
  source 1..59: mol_type = other DNA
gtgactggagttcagacgtgtgctcttccgatctcaggacctccacctctgagctattg

SEQ ID NO: 735 - DNA, 60 bp, synthetic construct
  source 1..60: mol_type = other DNA
gtgactggagttcagacgtgtgctcttccgatctagcccgacgcgtacaggatgatgtcc

SEQ ID NO: 736 - DNA, 55 bp, synthetic construct
  source 1..55: mol_type = other DNA
gtgactggagttcagacgtgtgctcttccgatctcttgccggaagagcctgactc

SEQ ID NO: 737 - DNA, 54 bp, synthetic construct
  source 1..54: mol_type = other DNA
gtgactggagttcagacgtgtgctcttccgatcttggaggcgctggacaaggtc

SEQ ID NO: 738 - DNA, 60 bp, synthetic construct
  source 1..60: mol_type = other DNA
gtgactggagttcagacgtgtgctcttccgatctctttccattctctgctggatgacgtg

SEQ ID NO: 739 - DNA, 59 bp, synthetic construct
  source 1..59: mol_type = other DNA
gtgactggagttcagacgtgtgctcttccgatctgagtatgcctgccgtgtgaaccatg

SEQ ID NO: 740 - DNA, 60 bp, synthetic construct
  source 1..60: mol_type = other DNA
gtgactggagttcagacgtgtgctcttccgatctgccagtaggtcatgaatataaagaca

SEQ ID NO: 741 - DNA, 60 bp, synthetic construct
  source 1..60: mol_type = other DNA
gtgactggagttcagacgtgtgctcttccgatctactctaccacatataggtccttggga

SEQ ID NO: 742 - DNA, 60 bp, synthetic construct
  source 1..60: mol_type = other DNA
gtgactggagttcagacgtgtgctcttccgatctgtgactggatccacaaccaaaattct

SEQ ID NO: 743 - DNA, 55 bp, synthetic construct
  source 1..55: mol_type = other DNA
gtgactggagttcagacgtgtgctcttccgatctcctgaggaaaaccatacagct

SEQ ID NO: 744 - DNA, 59 bp, synthetic construct
  source 1..59: mol_type = other DNA
gtgactggagttcagacgtgtgctcttccgatctatgccttggtgtagcactgacattc

SEQ ID NO: 745 - DNA, 60 bp, synthetic construct
  source 1..60: mol_type = other DNA
gtgactggagttcagacgtgtgctcttccgatctagtttgtatcttggatgccacatttt

SEQ ID NO: 746 - DNA, 60 bp, synthetic construct
  source 1..60: mol_type = other DNA
gtgactggagttcagacgtgtgctcttccgatctgcaccaagtcttttgccatgatgcga

SEQ ID NO: 747 - DNA, 54 bp, synthetic construct
  source 1..54: mol_type = other DNA
gtgactggagttcagacgtgtgctcttccgatctgggtgtcaccaaggccatgg

SEQ ID NO: 748 - DNA, 56 bp, synthetic construct
  source 1..56: mol_type = other DNA
gtgactggagttcagacgtgtgctcttccgatctaccatgtagggtcttgaatgag

SEQ ID NO: 749 - DNA, 58 bp, synthetic construct
  source 1..58: mol_type = other DNA
gtgactggagttcagacgtgtgctcttccgatctattgagtgagccccaagaatgacc

SEQ ID NO: 750 - DNA, 60 bp, synthetic construct
  source 1..60: mol_type = other DNA
gtgactggagttcagacgtgtgctcttccgatcttcacatcgtccccctttttaggtaga

SEQ ID NO: 751 - DNA, 57 bp, synthetic construct
  source 1..57: mol_type = other DNA
gtgactggagttcagacgtgtgctcttccgatctccctcgcacaacaaagggcttcc

SEQ ID NO: 752 - DNA, 53 bp, synthetic construct
  source 1..53: mol_type = other DNA
gtgactggagttcagacgtgtgctcttccgatctacgagcagggctggggaga

SEQ ID NO: 753 - DNA, 55 bp, synthetic construct
  source 1..55: mol_type = other DNA
gtgactggagttcagacgtgtgctcttccgatctgtgggctgtgggcacttctgc

SEQ ID NO: 754 - DNA, 55 bp, synthetic construct
  source 1..55: mol_type = other DNA
gtgactggagttcagacgtgtgctcttccgatctgaaggatgcccaggaggagca

SEQ ID NO: 755 - DNA, 53 bp, synthetic construct
  source 1..53: mol_type = other DNA
gtgactggagttcagacgtgtgctcttccgatcttgcagcgggggagtgtgta

SEQ ID NO: 756 - DNA, 55 bp, synthetic construct
  source 1..55: mol_type = other DNA
gtgactggagttcagacgtgtgctcttccgatctggtacacggcagggtcagggt

SEQ ID NO: 757 - DNA, 54 bp, synthetic construct
  source 1..54: mol_type = other DNA
gtgactggagttcagacgtgtgctcttccgatctagcgacctcgggtgggaaca

SEQ ID NO: 758 - DNA, 59 bp, synthetic construct
  source 1..59: mol_type = other DNA
gtgactggagttcagacgtgtgctcttccgatctcgatgtcccggcgcttgatgtggtg